(19) United States
(12) Patent Application Publication
(10) Pub. No.: US 2009/0155283 A1
(43) Pub. Date: Jun. 18, 2009

(54) Noncompetitive Domain Antibody Formats That Bind Interleukin 1 Receptor Type 1

(76) Inventors: Philip D. Drew, Cambridge (GB); Rudolf M.T. de Wildt, Cambridge (GB); Ian M. Tomlinson, Great Shelford (GB); Amrik Basran, Cambridge (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/085,920

(22) PCT Filed: Nov. 30, 2006
(86) PCT No.: PCT/GB2006/004471

§ 371 Date: Jul. 21, 2008

Related U.S. Application Data
(60) Provisional application No. 60/742,062, filed on Dec. 1, 2005.

(51) Int. Cl. A61K 39/395; C07K 16/28; C07H 21/04; C12N 15/63; C12N 5/10; C12P 21/04; A61K 9/12

(52) U.S. Cl. 424/143.1; 530/388.22; 536/23.53; 435/320.1; 435/325; 435/69.6; 424/45

(57) ABSTRACT

The invention relates to dAb monomers that bind IL-1R1 and inhibit binding of IL-1 (e.g., IL-1α and/or IL-1β) to the receptor but do not inhibit binding of IL-1ra to IL-1R1, and to ligands comprising such dAb monomers. The invention relates to protease resistand dAb monomers, and to ligands comprising protease resistant dAb monomers. The invention also relates to nucleic acids including vectors that encode the dAb monomers and ligand, to host cells that comprise the nucleic acids and to method for producing a dAb monomer or ligand. The invention also relates to pharmaceutical compositions that comprise the dAb monomers or ligands, and to therapeutic methods that comprise administering a dAb monomer of ligand.

### RELATED APPLICATION

This application claims the benefit of U.S. Provisional Application No. 60/742,062, filed on Dec. 1, 2005. The entire teachings of the above application are incorporated herein by reference.

### BACKGROUND OF THE INVENTION

Interleukin 1 (IL-1) is an important mediator of the immune response that has biological effects on several types of cells. Interleukin 1 binds to two receptors Interleukin 1 Receptor type 1 (IL-1R1, CD121a, p80), which transduces signal into cells upon binding IL-1, and Interleukin 1 Receptor type 2 (IL-1R1, CDw121b), which does not transduce signals upon binding IL-1 and acts as an endogenous regulator of IL-1. Another endogenous protein that regulates the interaction of IL-1 with IL-1R1 is Interleukin 1 receptor antagonist (IL-1ra). IL-1ra binds IL-1R1, but does not activate IL-1R1 to transduce signals.

Signals transduced through IL-1R1 upon binding IL-1 (e.g., IL-1α or IL-1β) induce a wide spectrum of biological activities that can be pathogenic. For example, signals transduced through IL-1R1 upon binding of IL-1 can lead to local or systemic inflammation, the elaboration of additional inflammatory mediators (e.g., IL-6, II-8, TNF), fever, activate immune cells (e.g., lymphocytes, neutrophils), anorexia, hypotension, leucopenia, and thrombocytopenia. Signals transduced through IL-1R1 upon binding of IL-1 also have effects on non-immune cells, such as stimulating chondrocytes to release collagenase and other enzymes that degrade cartilage, and stimulating the differentiation of osteoclast progenitor cells into mature osteoclasts which leads to resorption of bone. (See, e.g., Hallegua and Weisman, Ann. Theum. Dis. 61:960-967 (2002).) Accordingly, the interaction of IL-1 with IL-1R1 has been implicated in the pathogenesis of several diseases such as arthritis (e.g., rheumatoid arthritis, osteoarthritis) and inflammatory bowel disease.

Certain agents that bind Interleukin 1 Receptor Type 1 (IL-1R1) and neutralize its activity (e.g., IL-1ra) have proven to be effective therapeutic agents for certain inflammatory conditions, such as moderately to severely active rheumatoid arthritis. However, other agents that bind IL-1R1, such as the anti-IL-1R1 antibody AMG 108 (Amgen) have failed to meet primary endpoints in clinical studies.

A need exists for improved agents that antagonize IL-1R1 and methods for administering such agents to disease.

### SUMMARY OF THE INVENTION

The invention relates to domain antibody (dAb) monomers that bind IL-1R1 and inhibit binding of IL-1 (e.g., IL-1α and/or IL-1β) to the receptor but do not inhibit binding of IL-1ra to IL-1R1, and to ligands comprising such dAb monomers. Such ligands and dAb monomers are useful as therapeutic agents for treating inflammation, disease or other conditions mediated in whole or in part by biological functions induced by binding of IL-1 to IL-1R1 (e.g., local or systemic inflammation, elaboration of inflammatory mediators (e.g., IL-6, Il-8, TNF), fever, activation of immune cells (e.g., lymphocytes, neutrophils), anorexia, hypotension, leucopenia, thrombocytopenia.) The ligands or dAb monomers of the invention can bind IL-1R1 and inhibit IL-1R1 function without interfering with endogenous IL-1R1 inhibitory pathways, such as binding of endogenous IL-1ra to endogenous IL-1R1. Accordingly, such a ligand or dAb monomer can be administered to a subject to complement the endogenous regulatory pathways that inhibit the activity of IL-1R1 or IL-1 in vivo. In addition, ligands or dAb monomers that bind IL-1R1 and do not inhibit binding of IL-1ra to IL-1R1 provide advantages for use as diagnostic agents, because they can be used to bind and detect, quantify or measure IL-1R1 in a sample and will not compete with IL-1ra in the sample for binding to IL-1R1. Accordingly, an accurate determination of whether or how much IL-1R1 is in the sample can be made.

dAb monomers that bind IL-1R1 and inhibit binding of IL-1 (e.g., IL-1α and/or IL-1β) to the receptor but do not inhibit binding of IL-1ra to IL-1R1 also are useful as research tools. For example, such a dAb monomer can be used to identify agents (e.g., other dAbs, small organic molecules) that bind IL-1R1 and but do not inhibit binding of IL-1ra to IL-1R1. In one illustrative example, an agent or collection of agents to be tested for the ability to inhibit binding of IL-1 to IL-1R1 are assayed in a competitive IL-1R1 receptor binding assay, such as the receptor binding assay described herein. Agents that inhibit binding of IL-1 to IL-1R1 in such an assay can then be studied in a similar competitive IL-1R1 receptor binding assay to see if they compete with a dAb monomer that binds IL-1R1 but does not inhibit binding of IL-1ra to IL-1R1. Competitive binding in such an assay indicates that the agent binds IL-1R1 and inhibits binding of IL-1 to the receptor but does not inhibit binding of IL-1ra to the receptor.

In one aspect, the invention relates to a dAb monomer that has binding specificity for Interleukin-1 Receptor Type 1 (IL-1R1) and inhibits binding of Interleukin-1 (IL-1, e.g., Interleukin-1α (IL-1α) and/or Interleukin-1β (IL-1β)) to the receptor but does not inhibit binding of Interleukin-1 Receptor Antagonist (IL-1ra) to IL-1R1.

Preferably, the dAb monomer inhibits binding of IL-1 to IL-1R1 with an IC50 that is ≦1 μM. In some embodiments, the dAb monomer inhibits IL-1-induced release of Interleukin-8 by MRC-5 cells (ATCC Accession No. CCL-171) in an in vitro assay with a ND50 that is ≦1 μM, or preferably ≦1 nM. In other embodiments, the dAb monomer inhibits IL-1-induced release of Interleukin-6 in a whole blood assay with a ND50 that is ≦1 μM. In other embodiments, the dAb monomer inhibits IL-1-induced release of Interleukin-6 in a whole blood assay with a ND50 that is ≦1 μM.

One or more of the framework regions (FR) in the dAb monomer can comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat.

The amino acid sequences of one or more framework regions in the dAb monomer can be the same as the amino acid sequence of a corresponding framework region encoded by a human germline antibody gene segment, or the amino acid sequences of one or more of said framework regions collectively comprise up to 5 amino acid differences relative to the corresponding framework regions encoded by a human germline antibody gene segment.

The amino acid sequences of FR1, FR2, FR3 and FR4 in the dAb monomer can be the same as the amino acid sequences of corresponding framework regions encoded by a human germline antibody gene segment, or the amino acid sequences of FR1, FR2, FR3 and FR4 collectively contain up to 10 amino acid differences relative to the corresponding framework regions encoded by a human germline antibody gene segment.

The dAb monomer can comprise FR1, FR2 and FR3 regions, and the amino acid sequence of said FR1, FR2 and FR3 can be the same as the amino acid sequences of corresponding framework regions encoded by a human germline antibody gene segment. In some embodiments, the human germline antibody gene segment is DPK9 and JK1

In some embodiments, the dAb monomer competes for binding to IL-1R1 with a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173,) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-131 (SEQ ID NO:347), DOM4-132 (SEQ ID NO:348), and DOM4-133 (SEQ ID NO:349).

Preferably, the dAb monomer competes for binding to IL-1R1 with a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106); DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), and DOM4-122-73 (SEQ ID NO:165).

In other embodiments, the dAb monomer comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-1511 (SEQ ID NO:84), DOM4-112 (SEQ ID NO:85), DOM4-113 (SEQ ID NO:86), DOM4-114 (SEQ ID NO:87), DOM4-115 (SEQ ID NO:98), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173,) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-131 (SEQ ID NO:347), DOM4-132 (SEQ ID NO:348), and DOM4-133 (SEQ ID NO:349).

Preferably, the dAb monomer comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), and DOM4-122-73 (SEQ ID NO:165).

Preferably, the dAb monomer binds human IL-1R1 with an affinity (KD) of about 300 nM to about 5 pM, as determined by surface plasmon resonance.

In another aspect, the invention relates to a ligand comprising a dAb monomer that has binding specificity for Interleukin-1 Receptor Type 1 (IL-1R1) and inhibits binding of Interleukin-1 (IL-1, e.g., Interleukin-1α (IL-1α) and/or Interleukin-1β (IL-1β)) to the receptor but does not inhibit binding of Interleukin-1 Receptor Antagonist (IL-1ra) to IL-1R1, and a half-life extending moiety. The half-life extending moiety can be a polyalkylene glycol moiety, serum albumin or a fragment thereof, transferrin receptor or a transferrin-binding portion thereof, or an antibody or antibody fragment comprising a binding site for a polypeptide that enhances half-life in vivo. In some embodiments, the half-life extending moiety is an antibody or antibody fragment comprising a binding site for serum albumin or neonatal Fc receptor. In particular embodiments, the half-life extending moiety is an immunoglobulin single variable domain that competes with an anti-serum albumin dAb disclosed herein for binding to human serum albumin. In other particular embodiments, the half-life extending moiety is an immunoglobulin single variable domain that comprises an amino acid sequence that has at least 90% amino acid sequence identity with the amino acid sequence of an anti-serum albumin dAb disclosed herein.

In more particular embodiments, the invention is a ligand comprising a dAb monomer that has binding specificity for IL-1R1 and inhibits binding of IL-1 to the receptor but does not inhibit binding of IL-1ra to IL-1R1, wherein said dAb monomer is selected from the group consisting of DOM4-122-23, and DOM4-122-24. The ligand can be, for example, a dAb monomer, or a homodimer, homotrimer or homooligomer of said dAb monomer. The ligand can further comprise a dAb monomer that binds serum albumin, such as DOM7h-8. For example, in some embodiments, the ligand comprises DOM4-122-23 and DOM7h-8, or comprises DOM-122-24 and DOM7h-8.

In other particular embodiments, the invention is a ligand comprising a dAb monomer that has binding specificity for IL-1R1 and inhibits binding of IL-1 to the receptor but does not inhibit binding of IL-1ra to IL-1R1, and a dAb monomer that has binding specificity for tumor necrosis factor receptor 1 (TNFR1). If desired, the ligand can further comprise a half-life extending moiety.

Preferably, the dAb monomer that has binding specificity for TNFR1 competes for binding to TNFR1 with an anti-TNFR1 dAb described herein. In some embodiments, the dAb monomer that has binding specificity for TNFR1 comprises an amino acid sequence that has at least about 90% amino acid sequence identity with an amino acid sequence of an anti-TNFR1 dAb described herein.

The invention also relates to an isolated or recombinant nucleic acid encoding a dAb monomer or ligand, and to vectors (e.g., expression vectors) that comprise the recombinant nucleic acid. The invention also relates to a host cell comprising a recombinant nucleic acid or vector, and to a method of producing a ligand or dAb monomer that comprises maintaining a host cell of the invention under conditions suitable for expression of the nucleic acid that encodes a ligand or dAb monomer of the invention.

The invention also relates to pharmaceutical compositions comprising a dAb monomer or ligand and a physiologically acceptable carrier. For example, a pharmaceutical composition for intravenous, intramuscular, intraperitoneal, intraarterial, intrathecal, intraarticular, subcutaneous. pulmonary, intranasal, vaginal, or rectal administration.

The invention also relates to a drug delivery device comprising the pharmaceutical composition of the invention. For example, the drug delivery device can be a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, or rectal delivery device. Examples of such delivery devices, include a syringe, a transdermal delivery device (e.g., a patch), a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

The invention also relates to a method for treating an inflammatory disease comprising administering to a subject in need thereof, a therapeutically effective amount of a dAb monomer or ligand of the invention.

The invention also relates to a dAb monomer or ligand of the invention for use in therapy, diagnosis and/or prophylaxis, and to the use of a dAb monomer or ligand of the invention for the manufacture of a medicament for treating a disease described herein (e.g., an inflammatory disease, arthritis, a respiratory disease).

The invention also relates to a method for treating a disease (e.g., an inflammatory disease, arthritis, a respiratory disease) comprising administering to a subject in need thereof a therapeutically effective amount of a dAb monomer that is resistant to protease degradation.

The invention also relates a dAb monomer that is resistant to protease degradation for use in therapy, diagnosis or prophylaxis, and to the use of such a dAb monomer of the invention for the manufacture of a medicament for treating a disease described herein (e.g., an inflammatory disease, arthritis, a respiratory disease).

### BRIEF DESCRIPTION OF THE DRAWINGS

FIG. 1 is a graph showing the results of an in vitro assay in which dAbs were tested for the ability to inhibit IL-1-induced IL-8 release from cultured MRC-5 cells (ATCC catalogue no. CCL-171). FIG. 1 shows a dose-response curve for anti-IL-1R1 dAbs referred to as DOM4-122 and DOM4-129 in such a cell assay. The ND₅₀ values of both dAbs was about 1 μM.

FIG. 2 is a graph showing the results of in vitro assays in which dAbs that underwent affinity maturation were tested for the ability to inhibit IL-1-induced IL-8 release from cultured MRC-5 cells (ATCC catalogue no. CCL-171). FIG. 2 shows a dose-response curve for DOM4-122-6, DOM4-129-1, DOM4-122-23, and IL-1ra. DOM4-122-6 and DOM4-122-23 are affinity matured variants of DOM4-122, and DOM4-129-1 is an affinity matured variant of DOM4-129. Both DOM4-122-6 and DOM4-129-1 had an ND₅₀ of about 10 nM in the assay, and DOM4-122-23 had an ND₅₀ of approximately 1 nM in the assay.

FIGS. 3A and 3B are sensograms showing that DOM4-122-23 (FIG. 3A) but not IL-1α (FIG. 3B) bound to IL-1R1 to which IL-1ra was already bound. IL-1ra was injected over immobilized IL-1R1 and bound to the immobilized receptor. (Injection 1, from 0-60 seconds in FIGS. 3A and 3B.) Then, either DOM4-122-23 or IL-1α was injected. (Injection 2, from 60-120 seconds in FIGS. 3A and 3B.) As seen in the sensograms, DOM4-122-23 bound to IL-1R1 to which IL-1ra was already bound, but IL-1α did not.

FIG. 4 is a graph showing that increasing concentrations of DOM4-122-23 did not inhibit binding of IL-1ra to IL-1R1 in a competitive binding ELISA, but that IL-1α inhibited binding of IL-1ra to IL-1R1 in the assay. Increasing concentrations of DOM4-122-23 or IL-1α were mixed with 500 pM IL-1ra, and the mixture was applied to an ELISA plate that was coated with IL-1R1.

FIG. 5A-5Z illustrates the amino acid sequences of several human dAbs that bind human IL-1R1. In some of the sequences, the amino acids of CDR1, CDR2 and CDR3 are underlined.

FIGS. 6A-6Z, 6AA-6ZZ, 6AAA and 6BBB illustrates the nucleotide sequences of nucleic acids that encode the human dAbs shown in FIG. 5A-5Z. In some of the sequences, the nucleotides encoding CDR1, CDR2 and CDR3 are underlined.

FIG. 7A is an alignment of the amino acid sequences of three Vκs selected by binding to mouse serum albumin (MSA). The aligned amino acid sequences are from Vκs designated MSA16, which is also referred to as DOM7m-16 (SEQ ID NO:723), MSA 12, which is also referred to as DOM7m-12 (SEQ ID NO:724), and MSA 26, which is also referred to as DOM7m-26 (SEQ ID NO:725).

FIG. 7B is an alignment of the amino acid sequences of six Vκs selected by binding to rat serum albumin (RSA). The aligned amino acid sequences are from Vκs designated DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), and DOM7r-8 (SEQ ID NO:731).

FIG. 7C is an alignment of the amino acid sequences of six Vκs selected by binding to human serum albumin (HSA). The aligned amino acid sequences are from Vκs designated DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), and DOM7h-7 (SEQ ID NO:737).

FIG. 7D is an alignment of the amino acid sequences of seven V_{H}s selected by binding to human serum albumin and a consensus sequence (SEQ ID NO:738). The aligned sequences are from V_{H}s designated DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745).

FIG. 7E is an alignment of the amino acid sequences of three Vκs selected by binding to human serum albumin and rat serum albumin. The aligned amino acid sequences are from Vκs designated DOM7h-8 (SEQ ID NO:746), DOM7r-13 (SEQ ID NO:747), and DOM7r-14 (SEQ ID NO:748).

FIG. 8 is an illustration of the amino acid sequences of Vκs selected by binding to rat serum albumin (RSA). The illustrated sequences are from Vκs designated DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753).

FIG. 9A-9B is an illustration of the amino acid sequences of the amino acid sequences of V_{H}s that bind rat serum albumin (RSA). The illustrated sequences are from V_{H}s designated DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

FIG. 10 illustrates the amino acid sequences of several Camelid V_{HH}s that bind mouse serum albumin that are disclosed in WO 2004/041862. Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), Sequence Q (SEQ ID NO:784).

FIG. 11A-11V illustrates the amino acid sequences of several human immunoglobulin variable domains that have binding specificity for human TNFR1. The presented amino acid sequences are continuous with no gaps; the symbol ˜ has been inserted into the sequences to indicate the locations of the complementarity determining regions (CDRs). CDR1 is flanked by ˜, CDR2 is flanked by ˜˜, and CDR3 is flanked by ˜˜˜.

FIG. 12A-12B illustrates the amino acid sequences of several human immunoglobulin variable domains that have binding specificity for mouse TNFR1. The presented amino acid sequences are continuous with no gaps; the symbol ˜ has been inserted into some of the sequences to indicate the locations of the complementarity determining regions (CDRs). CDR1 is flanked by ˜, CDR2 is flanked by ˜˜, and CDR3 is flanked by ˜˜˜.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

As used herein, the term “ligand” refers to a polypeptide that comprises a domain that has binding specificity for a desired target. Preferably the binding domain is an immunoglobulin single variable domain (e.g., V_{H}, V_{L}, V_{HH}) that has binding specificity for a desired target antigen (e.g., a receptor protein). The binding domain can also comprises one or more complementarity determining regions (CDRs) of an immunoglobulin single variable domain that has binding specificity for a desired target antigen in a suitable format, such that the binding domain has binding specificity for the target antigen. For example, the CDRs can be grafted onto a suitable protein scaffold or skeleton, such as an affibody, an SpA scaffold, an LDL receptor class A domain or an EGF domain. Further, the ligand can be monovalent (e.g., a dAb monomer), bivalent (homobivalent, heterobivalent) or multivalent (homomultivalent, heteromultivalent) as described herein. Thus, “ligands” include polypeptides that consist of a dAb, include polypeptides that consist essentially of such a dAb, polypeptides that comprise a dAb (or the CDRs of a dAb) in a suitable format, such as an antibody format (e.g., IgG-like format, scFv, Fab, Fab′, F(ab′)₂) or a suitable protein scaffold or skeleton, such as an affibody, an SpA scaffold, an LDL receptor class A domain or an EGF domain, dual specific ligands that comprise a dAb that binds a first target protein, antigen or epitope (e.g., IL-1R1 or TNFR1) and a second dAb that binds another target protein, antigen or epitope (e.g., serum albumin), and multispecific ligands as described herein. The binding domain can also be a protein domain comprising a binding site for a desired target, e.g., a protein domain is selected from an affibody, an SpA domain, an LDL receptor class A domain an EGF domain, an avimer (see, e.g., U.S. Patent Application Publication Nos. 2005/0053973, 2005/0089932, 2005/0164301).

The phrase “immunoglobulin single variable domain” refers to an antibody variable region (V_{H}, V_{HH}, V_{L}) that specifically binds an antigen or epitope independently of other V regions or domains; however, as the term is used herein, an immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, preferably human, but also includes rodent (for example, as disclosed in WO 00/29004, the contents of which are incorporated herein by reference in their entirety) or camelid V_{HH} dAbs. Camelid dAbs are immunoglobulin single variable domain polypeptides which are derived from species including camel, llama, alpaca, dromedary, and guanaco, and comprise heavy chain antibodies naturally devoid of light chain: V_{HH}. V_{HH} molecules are about ten times smaller than IgG molecules, and as single polypeptides, they are very stable, resisting extreme pH and temperature conditions.

As used herein, the term “dose” refers to the quantity of agent (e.g., anti-1L-1R1 dAb, antagonist of TNFR1) administered to a subject all at one time (unit dose), or in two or more administrations over a defined time interval. For example, dose can refer to the quantity of agent (e.g., anti-1L-1R1 dAb, antagonist of TNFR1) administered to a subject over the course of one day (24 hours) (daily dose), two days, one week, two weeks, three weeks or one or more months (e.g., by a single administration, or by two or more administrations). The interval between doses can be any desired amount of time.

Two immunoglobulin domains are “complementary” when they belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on (for example) an immunoglobulin domain and a fibronectin domain are not complementary.

“Immunoglobulin” refers to a family of polypeptides which retain the immunoglobulin fold characteristic of antibody molecules, which contains two P sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules which possess binding domains. Preferably, the present invention relates to antibodies.

A “domain” is a folded protein structure which retains its tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

The term “repertoire” refers to a collection of diverse variants, for example polypeptide variants, which differ in their primary sequence. A library used in the present invention will encompass a repertoire of polypeptides comprising at least 1000 members.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

An “antibody” (for example IgG, IgM, IgA, IgD or IgE) or fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

A “dual-specific ligand” is a ligand comprising a first immunoglobulin single variable domain and a second immunoglobulin single variable domain as herein defined, wherein the variable regions are capable of binding to two different antigens or two epitopes on the same antigen which are not normally bound by a monospecific immunoglobulin. For example, the two epitopes may be on the same hapten, but are not the same epitope or sufficiently adjacent to be bound by a monospecific ligand. The dual specific ligands according to the invention are composed of variable domains which have different specificities, and do not contain mutually complementary variable domain pairs which have the same specificity. Dual-specific ligands and suitable methods for preparing dual-specific ligands are disclosed in WO 2004/058821, WO 2004/003019, and WO 03/002609, the entire teachings of each of these published international applications are incorporated herein by reference.

An “antigen” is a molecule that is bound by a ligand according to the present invention. Typically, antigens are bound by antibody ligands and are capable of raising an antibody response in vivo. It may be a polypeptide, protein, nucleic acid or other molecule. Generally, the dual specific ligands according to the invention are selected for target specificity against a particular antigen. In the case of conventional antibodies and fragments thereof, the antibody binding site defined by the variable loops (L1, L2, L3 and H1, H2, H3) is capable of binding to the antigen.

An “epitope” is a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity though variation in the hypervariable regions alone.

“Half-life” is the time taken for the serum concentration of the ligand to reduce by 50%, in vivo, for example due to degradation of the ligand and/or clearance or sequestration of the ligand by natural mechanisms. The ligands of the invention are stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus, a ligand specific for HSA and a target molecule is compared with the same ligand wherein the specificity for HSA is not present, that it does not bind HSA but binds another molecule. For example, it may bind a second epitope on the target molecule. Typically, the half life is increased by 10%, 20%, 30%, 40%, 50% or more. Increases in the range of 2×, 3×, 4×, 5×, 10×, 20×, 30×, 40×, 50× or more of the half life are possible. Alternatively, or in addition, increases in the range of up to 30×, 40×, 50×, 60×, 70×, 80×, 90×, 100×, 150× of the half life are possible.

As referred to herein, the term “competes” means that the binding of a first epitope to its cognate epitope binding domain is inhibited when a second epitope is bound to its cognate epitope binding domain. For example, binding may be inhibited sterically, for example by physical blocking of a binding domain or by alteration of the structure or environment of a binding domain such that its affinity or avidity for an epitope is reduced.

Amino acid and nucleotide sequence alignments and homology, similarity or identity, as defined herein are preferably prepared and determined using the algorithm BLAST 2 Sequences, using default parameters (Tatusova, T. A. et al, FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8

The invention relates to dAb monomers that bind IL-1R1 and inhibit binding of IL-1 (e.g., IL-1α and/or IL-1 β) to the receptor but do not inhibit binding of IL-1ra to IL-1R1, and to ligands comprising such dAb monomers. Such ligands and dAb monomers are useful as therapeutic agents for treating inflammation, disease or other conditions mediated in whole or in part by biological functions induced by binding of IL-1 to IL-1R1 (e.g., local or systemic inflammation, elaboration of inflammatory mediators (e.g., IL-6,1′-8, TNF), fever, activation of immune cells (e.g., lymphocytes, neutrophils), anorexia, hypotension, leucopenia, thrombocytopenia.) The ligands or dAb monomers of the invention can bind IL-1R1 and inhibit IL-1R1 function without interfering with endogenous IL-1R1 inhibitory pathways, such as binding of endogenous IL-1ra to endogenous IL-1R1. Accordingly, such a ligand or dAb monomer can be administered to a subject to complement the endogenous regulatory pathways that inhibit the activity of IL-1R1 or IL-1 in vivo. In addition, ligands or dAb monomers that bind IL-1R1 and do not inhibit binding of IL-1ra to IL-1R1 provide advantages for use as diagnostic agents, because they can be used to bind and detect, quantify or measure IL-1R1 in a sample and will not compete with IL-1ra in the sample for binding to IL-1R1. Accordingly, an accurate determination of whether or how much IL-1R1 is in the sample can be made.

dAb monomers that bind IL-1R1 and inhibit binding of IL-1 (e.g., IL-1α and/or IL-1β) to the receptor but do not inhibit binding of IL-1ra to IL-1R1 also are useful as research tools. For example, such a dAb monomer can be used to identify agents (e.g., other dAbs, small organic molecules) that bind IL-1R1 but do not inhibit binding of IL-1ra to IL-1R1. In one illustrative example, an agent or collection of agents to be tested for the ability to inhibit binding of IL-1 to IL-1R1 are assayed in a competitive IL-1R1 receptor binding assay, such as the receptor binding assay described herein. Agents that inhibit binding of IL-1 to IL-1R1 in such an assay can then be studied in a similar competitive IL-1R1 receptor binding assay to see if they compete with a dAb monomer that binds IL-1R1 but does not inhibit binding of IL-1ra to IL-1R1. Competitive binding in such an assay indicates that the agent binds IL-1R1 and inhibits binding of IL-1 to the receptor but does not inhibit binding of IL-1ra to the receptor.

Ligands and dAb Monomers that Bind IL-1R1

The invention provides ligands that comprise a dAb (e.g., dual specific ligand comprising such a dAb, dAb monomer) that binds to IL-1R1 with a K_{d} of 300 nM to 5 pM (i.e., 3×10⁻⁷ to 5×10⁻¹²M), preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ S⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

Preferably, the ligand or dAb monomer inhibits binding of IL-1 (e.g., IL-1α and/or IL-1β) to IL-1R1, for example in a receptor binding assay, with an inhibitory concentration 50 (IC50) that is equal to or less than about 1 μM, for example an IC50 of about 500 nM to about 50 pM, preferably about 100 nM to about 50 pM, more preferably about 10 nM to about 100 pM, advantageously about 1 nM to about 100 pM; for example about 50 nM or less, preferably about 5 nM or less, more preferably about 500 pM or less, advantageously about 200 pM or less, and most preferably about 100 pM or less.

Preferably, the ligand or dAb binds human IL-1R1 and inhibits binding of human IL-1 (e.g., IL-1α and/or IL-1β) to human IL-1R1 and inhibits signaling through human IL-1R1 in response to IL-1 binding.

Preferably, the ligand or dAb monomer neutralizes (inhibits the activity of) IL-1 or IL-1R1 in a standard assay (e.g., IL-1-induced release of Interleukin-8 by MRC-5 cells, IL-1-induced release of Interleukin-6 by whole blood cells) with a neutralizing dose 50 (ND50) that is less than or equal to about 1 μM, for example an ND50 of about 500 nM to about 50 pM, preferably about 100 nM to about 50 pM, more preferably about 10 nM to about 100 pM, advantageously about 1 nM to about 100 pM; for example about 50 nM or less, preferably about 5 nM or less, more preferably about 500 pM or less, advantageously about 200 pM or less, and most preferably about 100 pM or less. For example, the ligand or dAb monomer can inhibit IL-1-induced (e.g., IL-1α- or IL-1β-induced) release of Interleukin-8 by MRC-5 cells (ATCC Accession No. CCL-171) in an in vitro assay with a ND50 that is ≦10 μM, ≦1 μM, ≦100 nM, ≦10 nM, ≦1 nM, ≦500 pM, ≦300 pM, ≦100 pM, or ≦10 pM. In another example, the ligand or dAb monomer can inhibit IL-1-induced (e.g., IL-1α- or IL-1β-induced) release of Interleukin-6 in an in vitro whole blood assay with a ND50 that is ≦10 μM, ≦1 μM, ≦100 nM, ≦10 nM, ≦1 nM, ≦500 pM, ≦300 pM, ≦100 pM, or ≦10 pM.

The ligand can be monovalent (e.g., a dAb monomer) or multivalent (e.g., dual specific, multi-specific) as described herein. In particular embodiments, the ligand is a dAb monomer that binds human IL-1R1 and comprises a half-life extending moiety (as described herein) such as a polyethylene glycol moiety.

In other embodiments, the ligand is multivalent and comprises two or more dAb monomers that bind IL-1R1. Multivalent ligands can contain two or more copies of a particular dAb that binds IL-1R1 or contain two or more dAbs that bind IL-1R1. For example, as described herein, the ligand can be a dimer, trimer or multimer comprising two or more copies of a particular dAb that binds IL-1R1, or can comprise two or more different dAbs that bind IL-1R1. In some examples, the ligand is a homo dimer or homo trimer that comprises two or three copies of a particular dAb that binds IL-1R1, respectively. Preferably, a multivalent ligand does not substantially agonize IL-1R1 (act as an agonist of IL-1R1) in a standard cell assay (i.e., when present at a concentration of 1 nM, 10 nM, 100 nM, 1 μM, 10 μM, 100 μM, 1000 μM or 5,000 μM, results in no more than about 5% of the IL-1R1-mediated activity induced by IL-1 (100 pg/ml) in the assay).

In certain embodiments, the multivalent ligand contains two or more dAbs that bind a desired epitope or domain of IL-1R1. For example, the multivalent ligand can comprise two or more copies of a dAb that competes with IL-1ra for binding to IL-1R1. In another example, the multivalent ligand can comprise two or more copies of a dAb that does not compete with IL-1 ra for binding to IL-1R1.

In other embodiments, the multivalent ligand contains two or more dAbs that bind to different epitopes or domains of IL-1R1. In one example, the multivalent ligand comprises a first dAb that binds a first epitope of IL-1R1, and a second dAb that binds a second different epitope of IL-1R1. Ligands of this type can bind IL-1R1 with high aviditiy, and be more selective for binding to cells that overexpress IL-1R1 or express IL-1R1 on their surface at high density than other ligand formats, such as dAb monomers.

In certain embodiments, the ligands or dAb monomers of the invention are efficacious in a model disease (e.g., inflammatory disease) when an effective amount is administered. Generally an effective amount in a model of inflammatory disease is about 1 mg/kg to about 10 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). The models of chronic inflammatory disease described herein are recognized by those skilled in the art as being predictive of therapeutic efficacy in humans. The prior art does not suggest using ligands or dAb monomers, as described herein, in these models, or that they would be efficacious.

Several suitable animal models of respiratory disease are known in the art, and are recognized by those skilled in the art as being predictive of therapeutic efficacy in humans. For example, suitable animal models of respiratory disease include models of chronic obstructive pulmonary disease (see, Groneberg, D A et al., Respiratory Research 5:18 (2004)), and models of asthma (see, Coffman et al, J. Exp. Med. 201 (12):1875-1879 (2001). For example, the ligand or dAb monomer can be efficacious in the mouse model of tobacco smoke-induced chronic obstructive pulmonary disease (COPD) (See, e.g., Wright J L and Churg A., Chest 122:301S-306S (2002)). For example, administering an effective amount of the ligand or dAb monomer can reduce or delay onset of the symptoms of COPD, as compared to a suitable control.

In particular embodiments, the ligand or dAb monomer is efficacious in a standard model of arthritis (e.g., inflammatory arthritis, osteoarthritis). Several suitable models are known in the art, for example, mouse collagen-induced arthritis model (see, e.g., Juarranz, et al., Arthritis Research and Therapy, 7:R11034-R1045 (2005)), rat adjuvant induced arthritis (see, e.g., Halloran, M. et al., J. Immunol., 65:7492 (1999), Halloran, M. et al., Arthritis Rheum., 39:810 (1996)), rabbit experimental osteoarthritis (see, e.g., Spriet, et al. Osteoarthritis and Cartilage, 13:171-179 (2005), and several mouse models of osteoarthritis (see, e.g., Helminen, et al., Rheumatology, 41:848-856 (2002)).

For example, arthritis can be induced in DBA/1 mice by injecting animals with an emulsion of Arthrogen-CIA adjuvant and Arthrogen-CIA collagen (MD-biosciences). About 21 days after the injection, and ligand or dAb monomer to be tested can be administered (e.g., by intraperitoneal injection). Clinical arthritic scores on a scale of 0 to 4 can be measured for each of the 4 limbs of the animals assigning 0 for a normal limb and assigning 4 for a maximally inflamed limb with involvement of multiple joints. Administering an effective amount of ligand or dAb monomer can reduce the average arthritic score of the summation of the four limbs in this mouse collagen-induced arthritis model, for example, the average arthritic score of the summation of the four limbs can be reduced by about 1 to about 16, about 3 to about 16, about 6 to about 16, about 9 to about 16, or about 12 to about 16, as compared to a suitable control, or can delay the onset of symptoms of arthritis, for example, by about 1 day, about 2 days, about 3 days, about 4 days, about 5 days, about 6 days, about 7 days, about 10 days, about 14 days, about 21 days or about 28 days, as compared to a suitable control. In another example, administering an effective amount of the ligand can result in an average arthritic score of the summation of the four limbs in the standard mouse collagen-induced arthritis model of 0 to about 3, about 3 to about 5, about 5 to about 7, about 7 to about 15, about 9 to about 15, about 10 to about 15, about 12 to about 15, or about 14 to about 15.

In other embodiments, the ligand or dAb monomer is efficacious in the mouse ΔARE model of arthritis (Kontoyiannis et al, J Exp Med 196:1563-74 (2002)). For example, administering an effective amount of the ligand can reduce the average arthritic score in the mouse ΔARE model of arthritis, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the ligand can delay the onset of symptoms of arthritis in the mouse ΔARE model of arthritis by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the ligand can result in an average arthritic score in the mouse ΔARE model of arthritis of 0 to about 0.5, about 0.5 to about 1, about 1 to about 1.5, about 1.5 to about 2, or about 2 to about 2.5.

In other embodiments, the ligand or dAb monomer is efficacious in the mouse ΔARE model of inflammatory bowel disease (IBD) (Kontoyiannis et al., J Exp Med 196:1563-74 (2002)). For example, administering an effective amount of the ligand can reduce the average acute and/or chronic inflammation score in the mouse ΔARE model of IBD, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the ligand can delay the onset of symptoms of IBD in the mouse ΔARE model of IBD by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the ligand can result in an average acute and/or chronic inflammation score in the mouse ΔARE model of IBD of 0 to about 0.5, about 0.5 to about 1, about 1 to about 1.5, about 1.5 to about 2, or about 2 to about 2.5.

In other embodiments, the ligand or dAb monomer is efficacious in the mouse dextran sulfate sodium (DSS) induced model of IBD (see, Okayasu I. et al., Gastroenterology 98:694-702 (1990); Podolsky K., J Gasteroenterol. 38 suppl XV:63-66 (2003)). For example, administering an effective amount of the ligand can reduce the average severity score in the mouse DSS model of IBD, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the ligand can delay the onset of symptoms of IBD in the mouse DSS model of IBD by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the ligand can result in an average severity score in the mouse DSS model of IBD of 0 to about 0.5, about 0.5 to about 1, about 1 to about 1.5, about 1.5 to about 2, or about 2 to about 2.5.

In some embodiments, the ligand comprises a dAb that specifically binds IL-1R1, inhibits binding of IL-1 (e.g., IL-1α and/or IL-1β) to the receptor but does not inhibit binding of IL-1 ra to IL-1R1, and competes for binding to IL-1R1 with dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), and DOM4-122-73 (SEQ ID NO:165).

In some embodiments, the ligand comprises a dAb that specifically binds IL-1R, inhibits binding of IL-1 (e.g., IL-1α and/or IL-1β) to the receptor but does not inhibit binding of IL-1ra to IL-1R1, and comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), and DOM4-122-73 (SEQ ID NO:165).

In some embodiments, the ligand comprises a dAb that binds IL-1R1 and competes with any of the dAbs disclosed herein for binding to IL-1R1 (e.g., human IL-1R1).

In preferred embodiments, the ligand comprises a dAb monomer selected from the group consisting of DOM4-122-23, and DOM4-122-24. For example, the ligand can be a monomer, or be a hetero- or homo-dimer, trimer or oligomer of these dAbs. If desired, the ligand can further comprise a half-life extending moiety, such as a polyethylene glycol moiety. In some embodiment, the ligand comprises a dAb monomer selected from the group consisting of DOM4-122-23, and DOM4-122-24, and a dAb monomer that binds serum albumin. For example, the ligand can be a dual specific ligand that comprises DOM4-122-23 and DOM7h-8, or DOM4-122-24 and DOM7h-8.

The dAb monomer can comprise any suitable immunoglobulin variable domain, and preferably comprises a human variable domain or a variable domain that comprises human framework regions. In certain embodiments, the dAb monomer comprises a universal framework, as described herein.

The universal framework can be a V_{L} framework (Vλ or Vκ), such as a framework that comprises the framework amino acid sequences encoded by the human germline DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprises the framework amino acid sequence encoded by the human germline J_{κ}1, J_{κ}2, J_{κ}3, J_{κ}4, or J_{κ}5 immunoglobulin gene segment.

In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segment. If desired, the V_{H} framework can further comprises the framework amino acid sequence encoded by the human germline J_{H}1, J_{H}2, J_{H}3, J_{H}4, J_{H}4b, J_{H}5 and J_{H}6 immunoglobulin gene segment.

In certain embodiments, the dAb monomer comprises one or more framework regions comprising an amino acid sequence that is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In other embodiments, the amino acid sequences of FW1, FW2, FW3 and FW4 of the dAb monomer are the same as the amino acid sequences of corresponding framework regions encoded by a human germline antibody gene segment, or the amino acid sequences of FW1, FW2, FW3 and FW4 collectively contain up to 10 amino acid differences relative to the amino acid sequences of corresponding framework regions encoded by said human germline antibody gene segment.

In other embodiments, the dAb monomer comprises FW1, FW2 and FW3 regions, and the amino acid sequence of said FW1, FW2 and FW3 regions are the same as the amino acid sequences of corresponding framework regions encoded by human germline antibody gene segments.

In particular embodiments, the dAb monomer ligand comprises the DPK9 V_{L} framework, or a V_{H} framework selected from the group consisting of DP47, DP45 and DP38. The dAb monomer can comprise a binding site for a generic ligand, such as protein A, protein L and protein G.

In certain embodiments, the ligand or dAb monomer is substantially resistant to aggregation. For example, in some embodiments, less than about 10%, less than about 9%, less than about 8%, less than about 7%, less than about 6%, less than about 5%, less than about 4%, less than about 3%, less than about 2% or less than about 1% of the ligand or dAb monomer aggregates when a 1-5 mg/ml, 5-10 mg/ml, 10-20 mg/ml, 20-50 mg/ml, 50-100 mg/ml, 100-200 mg/ml or 200-500 mg/ml solution of ligand or dAb in a solvent that is routinely used for drug formulation such as saline, buffered saline, citrate buffer saline, water, an emulsion, and any of these solvents with an acceptable excipient such as those approved by the FDA, is maintained at about 22° C., 22-25° C., 25-30° C., 30-37° C., 37-40° C., 40-50° C., 50-60° C., 60-70° C., 70-80° C., 15-20° C., 10-15° C., 5-10° C., 2-5° C., 0-2° C., −10° C. to 0° C., −20° C. to −10° C., −40° C. to −20° C., −60° C. to −40° C., or −80° C. to −60° C., for a period of about time, for example, 10 minutes, 1 hour, 8 hours, 24 hours, 2 days, 3 days, 4 days, 1 week, 2 weeks, 3 weeks, 1 month, 2 months, 3 months, 4 months, 6 months, 1 year, or 2 years.

Aggregation can be assessed using any suitable method, such as, by microscopy, assessing turbidity of a solution by visual inspection or spectroscopy or any other suitable method. Preferably, aggregation is assessed by dynamic light scattering. Ligands or dAb monomers that are resistant to aggregation provide several advantages. For example, such ligands or dAb monomers can readily be produced in high yield as soluble proteins by expression using a suitable biological production system, such as E. coli, and can be formulated and/or stored at higher concentrations than conventional polypeptides, and with less aggregation and loss of activity.

In addition, ligands or dAb monomers that are resistant to aggregation can be produced more economically than other antigen- or epitope-binding polypeptides (e.g., conventional antibodies). For example, generally, preparation of antigen- or epitope-binding polypeptides intended for in vivo applications includes processes (e.g., gel filtration) that remove aggregated polypeptides. Failure to remove such aggregates can result in a preparation that is not suitable for in vivo applications because, for example, aggregates of an antigen-binding polypeptide that is intended to act as an antagonist can function as an agonist by inducing cross-linking or clustering of the target antigen. Protein aggregates can also reduce the efficacy of therapeutic polypeptides by inducing an immune response in the subject to which they are administered.

In contrast, the aggregation resistant ligands or dAb monomers of the invention can be prepared for in vivo applications without the need to include process steps that remove aggregates, and can be used in in vivo applications without the aforementioned disadvantages caused by polypeptide aggregates.

In some embodiments, the ligand or dAb monomer unfolds reversibly when heated to a temperature (Ts) and cooled to a temperature (Tc), wherein Ts is greater than the melting temperature (Tm) of the dAb, and Tc is lower than the melting temperature of the dAb. For example, the dAb monomer can unfold reversibly when heated to 80° C. and cooled to about room temperature. A polypeptide that unfolds reversibly loses function when unfolded but regains function upon refolding. Such polypeptides are distinguished from polypeptides that aggregate when unfolded or that improperly refold (misfolded polypeptides), i.e., do not regain function.

Polypeptide unfolding and refolding can be assessed, for example, by directly or indirectly detecting polypeptide structure using any suitable method. For example, polypeptide structure can be detected by circular dichroism (CD) (e.g., far-UV CD, near-UV CD), fluorescence (e.g., fluorescence of tryptophan side chains), susceptibility to proteolysis, nuclear magnetic resonance (NMR), or by detecting or measuring a polypeptide function that is dependent upon proper folding (e.g., binding to target ligand, binding to generic ligand). In one example, polypeptide unfolding is assessed using a functional assay in which loss of binding function (e.g., binding a generic and/or target ligand, binding a substrate) indicates that the polypeptide is unfolded.

The extent of unfolding and refolding of a ligand or dAb monomer can be determined using an unfolding or denaturation curve. An unfolding curve can be produced by plotting temperature as the ordinate and the relative concentration of folded polypeptide as the abscissa. The relative concentration of folded ligand or dAb monomer can be determined directly or indirectly using any suitable method (e.g., CD, fluorescence, binding assay). For example, a ligand or dAb monomer solution can be prepared and ellipticity of the solution determined by CD. The ellipticity value obtained represents a relative concentration of folded ligand or dAb monomer of 100%. The ligand or dAb monomer in the solution is then unfolded by incrementally raising the temperature of the solution and ellipticity is determined at suitable increments (e.g., after each increase of one degree in temperature). The ligand or dAb monomer in solution is then refolded by incrementally reducing the temperature of the solution and ellipticity is determined at suitable increments. The data can be plotted to produce an unfolding curve and a refolding curve. The unfolding and refolding curves have a characteristic sigmoidal shape that includes a portion in which the ligand or dAb monomer molecules are folded, an unfolding/refolding transition in which ligand or dAb monomer molecules are unfolded to various degrees, and a portion in which the ligand or dAb monomer molecules are unfolded. The y-axis intercept of the refolding curve is the relative amount of refolded ligand or dAb monomer recovered. A recovery of at least about 50%, or at least about 60%, or at least about 70%, or at least about 75%, or at least about 80%, or at least about 85%, or at least about 90%, or at least about 95% is indicative that the ligand or dAb monomer unfolds reversibly.

In a preferred embodiment, reversibility of unfolding of the ligand or dAb monomer is determined by preparing a ligand or dAb monomer solution and plotting heat unfolding and refolding curves. The ligand or dAb monomer solution can be prepared in any suitable solvent, such as an aqueous buffer that has a pH suitable to allow the ligand or dAb monomer to dissolve (e.g., pH that is about 3 units above or below the isoelectric point (pI)). The ligand or dAb monomer solution is concentrated enough to allow unfolding/folding to be detected. For example, the ligand or dAb monomer solution can be about 0.1 μM to about 100 μM, or preferably about 1 μM to about 10 μM.

If the melting temperature (Tm) of the ligand or dAb monomer is known, the solution can be heated to about ten degrees below the Tm (Tm−10) and folding assessed by ellipticity or fluorescence (e.g., far-UV CD scan from 200 nm to 250 nm, fixed wavelength CD at 235 nm or 225 nm; tryptophan fluorescent emission spectra at 300 to 450 nm with excitation at 298 nm) to provide 100% relative folded ligand or dAb monomer. The solution is then heated to at least ten degrees above Tm (Tm+10) in predetermined increments (e.g., increases of about 0.1 to about 1 degree), and ellipticity or fluorescence is determined at each increment. Then, the ligand or dAb monomer is refolded by cooling to at least Tm−10 in predetermined increments and ellipticity or fluorescence determined at each increment. If the melting temperature of the ligand or dAb monomer is not known, the solution can be unfolded by incrementally heating from about 25° C. to about 100° C. and then refolded by incrementally cooling to at least about 25° C., and ellipticity or fluorescence at each heating and cooling increment is determined. The data obtained can be plotted to produce an unfolding curve and a refolding curve, in which the y-axis intercept of the refolding curve is the relative amount of refolded protein recovered. In some embodiments, the dAb monomer does not comprise a Camelid immunoglobulin variable domain, or one or more framework amino acids that are unique to immunoglobulin variable domains encoded by Camelid germline antibody gene segments.

Preferably, the ligand or dAb monomer is secreted in a quantity of at least about 0.5 mg/L when expressed in E. coli or in Pichia species (e.g., P. pastoris). In other preferred embodiments, the dAb monomer is secreted in a quantity of at least about 0.75 mg/L, at least about 1 mg/L, at least about 4 mg/L, at least about 5 mg/L, at least about 10 mg/L, at least about 15 mg/L, at least about 20 mg/L, at least about 25 mg/L, at least about 30 mg/L, at least about 35 mg/L, at least about 40 mg/L, at least about 45 mg/L, or at least about 50 mg/L, or at least about 100 mg/L, or at least about 200 mg/L, or at least about 300 mg/L, or at least about 400 mg/L, or at least about 500 mg/L, or at least about 600 mg/L, or at least about 700 mg/L, or at least about 800 mg/L, at least about 900 mg/L, or at least about 1 g/L when expressed in E. coli or in Pichia species (e.g., P. pastoris). In other preferred embodiments, the dAb monomer is secreted in a quantity of at least about 1 mg/L to at least about 1 g/L, at least about 1 mg/L to at least about 750 mg/L, at least about 100 mg/L to at least about 1 g/L, at least about 200 mg/L to at least about 1 g/L, at least about 300 mg/L to at least about 1 g/L, at least about 400 mg/L to at least about 1 g/L, at least about 500 mg/L to at least about 1 g/L, at least about 600 mg/L to at least about 1 g/L, at least about 700 mg/L to at least about 1 g/L, at least about 800 mg/L to at least about 1 g/L, or at least about 900 mg/L to at least about 1 g/L when expressed in E. coli or in Pichia species (e.g., P. pastoris). Although, the ligands and dAb monomers described herein can be secretable when expressed in E. coli or in Pichia species (e.g., P. pastoris), they can be produced using any suitable method, such as synthetic chemical methods or biological production methods that do not employ E. coli or Pichia species.

dAb Monomers that Bind Serum Albumin

The ligand of the invention can comprise a dAb monomer that binds serum albumin (SA) with a K_{d} of 1 nM to 500 μM (i.e., ×10⁻⁹ to 5×10⁻⁴), preferably 100 nM to 10 μM. Preferably, for a dual specific ligand comprising a first anti-SA dAb and a second dAb to another target, the affinity (e.g. K_{d} and/or K_{off} as measured by surface plasmon resonance, e.g. using BiaCore) of the second dAb for its target is from 1 to 100000 times (preferably 100 to 100000, more preferably 1000 to 100000, or 10000 to 100000 times) the affinity of the first dAb for SA. For example, the first dAb binds SA with an affinity of approximately 10 μM, while the second dAb binds its target with an affinity of 100 pM. Preferably, the serum albumin is human serum albumin (HSA). In one embodiment, the first dAb (or a dAb monomer) binds SA (e.g., HSA) with a K_{d} of approximately 50, preferably 70, and more preferably 100, 150 or 200 nM.

In certain embodiments, the dAb monomer that binds SA resists aggregation, unfolds reversibly and/or comprises a framework region as described above for dAb monomers that bind IL-1R1.

In particular embodiments, the antigen-binding fragment of an antibody that binds serum albumin is a dAb that binds human serum albumin. In certain embodiments, the dAb binds human serum albumin and competes for binding to albumin with a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

For example, the dAb that binds human serum albumin can comprise an amino acid sequence that has at least about 90%, or at least about 95%, or at least about 96%, or at least about 97%, or at least about 98%, or at least about 99% amino acid sequence identity with DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745).

Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In more particular embodiments, the dAb is a V_{κ} dAb that binds human serum albumin and has a amino acid sequence selected from the group consisting of DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), and DOM7r-14 (SEQ ID NO:748), or a V_{H} dAb that has an amino acid sequence selected from the group consisting of DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745). In other embodiments, the antigen-binding fragment of an antibody that binds serum albumin is a dAb that binds human serum albumin and comprises the CDRs of any of the foregoing amino acid sequences.

Suitable Camelid V_{HH} that bind serum albumin include those disclosed in WO 2004/041862 (Ablynx N.V.) and herein (SEQ ID NOS:768-784). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:780, SEQ ID NO:781, SEQ ID NO:782, SEQ ID NO:783, or SEQ ID NO:784. Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In some embodiments, the ligand comprises an anti-serum albumin dAb that competes with any anti-serum albumin dAb disclosed herein for binding to serum albumin (e.g., human serum albumin).

dAb Monomers that Bind Tumor Necrosis Factor Receptor 1 (TNFR1)

The ligand of the invention can comprise a dAb monomer that binds TNFR1. TNFR1 is a transmembrane receptor containing an extracellular region that binds ligand and an intracellular domain that lacks intrinsic signal transduction activity but can associate with signal transduction molecules. The complex of TNFR1 with bound TNF contains three TNFR1 chains and three TNF chains. (Banner et al., Cell, 73(3) 431-445 (1993).) The TNF ligand is present as a trimer, which is bound by three TNFR1 chains. (Id.) The three TNFR1 chains are clustered closely together in the receptor-ligand complex, and this clustering is a prerequisite to TNFR1-mediated signal transduction. In fact, multivalent agents that bind TNFR1, such as anti-TNFR1 antibodies, can induce TNFR1 clustering and signal transduction in the absence of TNF and are commonly used as TNFR1 agonists. (See, e.g., Belka et al., EMBO, 14(6):1156-1165 (1995); Mandik-Nayak et al., J. Immunol, 167:1920-1928 (2001).) Accordingly, multivalent agents that bind TNFR1, are generally not effective antagonists of TNFR1 even if they block the binding of TNFα to TNFR1.

The extracellular region of TNFR1 comprises a thirteen amino acid amino-terminal segment (amino acids 1-13 of SEQ ID NO:996 (human); amino acids 1-13 of SEQ ID NO:997 (mouse)), Domain 1 (amino acids 14-53 of SEQ ID NO:996 (human); amino acids 14-53 of SEQ ID NO:997 (mouse)), Domain 2 (amino acids 54-97 of SEQ ID NO:996 (human); amino acids 54-97 of SEQ ID NO:997 (mouse)), Domain 3 (amino acids 98-138 of SEQ ID NO:996 (human); amino acid 98-138 of SEQ ID NO:997 (mouse)), and Domain 4 (amino acids 139-167 of SEQ ID NO:996 (human); amino acids 139-167 of SEQ ID NO:997 (mouse)) which is followed by a membrane-proximal region (amino acids 168-182 of SEQ ID NO:996 (human); amino acids 168-183 SEQ ID NO:997 (mouse)). (See, Banner et al., Cell 73(3) 431-445 (1993) and Loetschler et al., Cell 61(2) 351-359 (1990).) Domains 2 and 3 make contact with bound ligand (TNFβ, TNFα). (Banner et al., Cell, 73(3) 431-445 (1993).) The extracellular region of TNFR1 also contains a region referred to as the pre-ligand binding assembly domain or PLAD domain (amino acids 1-53 of SEQ ID NO:996 (human); amino acids 1-53 of SEQ ID NO:997 (mouse)) (The Government of the USA, WO 01/58953; Deng et al., Nature Medicine, doi: 10.1038/nm 1304 (2005)).

TNFR1 is shed from the surface of cells in vivo through a process that includes proteolysis of TNFR1 in Domain 4 or in the membrane-proximal region (amino acids 168-182 of SEQ ID NO:213, amino acids 168-183 of SEQ ID NO:215, respectively), to produce a soluble form of TNFR1. Soluble TNFR1 retains the capacity to bind TNFα, and thereby functions as an endogenous inhibitor of the activity of TNFα.

The extracellular region of human TNFR1 has the following amino acid sequence.

(SEQ ID NO:996)

LVPHLGDREKRDSVCPQGKYIHPQNNSICCTKCHKGTYLYNDCPGPGQDT
DCRECESGSFTASENHLRHCLSCSKCRKEMGQVEISSCTVDRDTVCGCRK
NQYRHYWSENLFQCFNCSLCLNGTVHLSCQEKQNTVCTCHAGFFLRENEC
VSCSNCKKSLECTKLCLPQIENVKGTEDSGTT

The extracellular region of murine (Mus musculus) TNFR1 has the following amino acid sequence.

(SEQ ID NO:997)

LVPSLGDREKRDSLCPQGKYVHSKNNSICCTKCHKGTYLVSDCPSPGRDT
VCRECEKGTFTASQNYLRQCLSCKTCRKEMSQVEISPCQADKDTVCGCKE
NQFQRYLSETHFQCVDCSPCFNGTVTIPCKETQNTVCNCHAGFFLRESEC
VPCSHCKKNEECMKLCLPPPLANVTNPQDSGTA

Anti-TNFR1 dAbs suitable for use in the invention (e.g., ligands described herein) have binding specificity for Tumor Necrosis Factor Receptor 1 (TNFR1; p55; CD120a). Preferably the antagonists of TNFR1 do not have binding specificity for Tumor Necrosis Factor 2 (TNFR2), or do not substantially antagonize TNFR2. An antagonist of TNFR1 does not substantially antagonize TNFR2 when the antagonist (1 nM, 10 nM, 100 nM, 1 μM, 10 μM or 100 μM) results in no more than about 5% inhibition of TNFR2-mediated activity induced by TNFα (100 pg/ml) in a standard cell assay. In certain embodiments, the dAb monomer that binds TNFR1 resists aggregation, unfolds reversibly and/or comprises a framework region as described above for dAb monomers that bind IL-1R1.

Suitable anti-TNFR1 dAbs and ligands that comprise such dAbs, do not induce cross-linking or clustering of TNFR1 on the surface of cells which can lead to activation of the receptor and signal transduction. In particular embodiments, the ligand comprises an anti-TNFR1 dAb that binds to Domain 1 of TNFR1. In more particular embodiments, the ligand comprises an anti-TNFR1 dAb that binds to Domain 1 of TNFR1, and competes with TAR2m-21-23 for binding to mouse TNFR1 or competes with TAR2h-205 for binding to human TNFR1.

In certain embodiments, the anti-TNFR1 dAb binds Domain 2 and/or Domain 3 of TNFR1. In particular embodiments, the anti-TNFR1 dAb competes with TAR2h-10-27, TAR2h-131-8, TAR2h-15-8, TAR2h-35-4, TAR2h-154-7, TAR2h-154-10 or TAR2h-185-25 for binding to TNFR1 (e.g., human and/or mouse TNFR1).

Preferably, anti-TNFR1 dAb monomers suitable for use in the ligands of the invention bind TNFR1 with a K_{d} of 300 nM to 5 pM (i.e., 3×10⁻⁷ to 5×10⁻¹²M), preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance. (The K_{d}=K_{off}/Kₒₙ). Certain anti-TNFR1 dAb monomers suitable for use in the invention specifically bind human TNFR1 with a K_{d} of 50 nM to 20 pM, and a K_{off} rate constant of 5×10⁻¹ s⁻¹ to 1×10⁻⁷ s⁻¹, as determined by surface plasmon resonance.

Some anti-TNFR1 dAb monomers inhibit binding of TNFα to TNFR1. For example, some anti-TNFR1 dAb monomers inhibit binding of TNFα to TNFR1 with an inhibitory concentration 50 (IC50) of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNFR1 is human TNFR1.

Other anti-TNFR1 dAb monomers do not inhibit binding of TNFα to TNFR1, but inhibit signal transduction mediated through TNFR1. For example, an anti-TNFR1 dAb monomer can inhibit TNFα-induced clustering of TNFR1, which precedes signal transduction through TNFR1. For example, certain anti-TNFR1 dAb monomers can bind TNFR1 and inhibit TNFR1-mediated signaling, but do not substantially inhibit binding of TNFα to TNFR1. For example, the anti-TNFR1 dAb monomer inhibits TNFα-induced crosslinking or clustering of TNFR1 on the surface of a cell. Such dAbs (e.g., TAR2m-21-23 described herein) are advantageous because they can antagonize cell surface TNFR1 but do not substantially reduce the inhibitory activity of endogenous soluble TNFR1. For example, the anti-TNFR1 dAb can bind TNFR1, but inhibits binding of TNFα to TNFR1 in a receptor binding assay by no more that about 10%, no more that about 5%, no more than about 4%, no more than about 3%, no more than about 2%, or no more than about 1%. Also, in these embodiments, the anti-TNFR1 dAb inhibits TNFα-induced crosslinking of TNFR1 and/or TNFR1-mediated signaling in a standard cell assay by at least about 10%, at least about 20%, at least about 30%, at least about 40%, at least about 50%, at least about 60%, at least about 70%, at least about 80%, at least about 90%, at least about 95%, or at least about 99%. Accordingly, administering a ligand that comprises such a dAb monomer to a mammal in need thereof can complement the endogenous regulatory pathways that inhibit the activity TNFα and the activity of TNFR1 in vivo.

Preferably, the ligand or dAb monomer neutralizes (inhibits the activity of) TNFR1 in a standard assay (e.g., the standard L929 or standard HeLa IL-8 assays described herein) with a neutralizing dose 50 (ND50) of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. In other embodiments, the anti-TNFR1 dAb monomer binds TNFRI and antagonizes the activity of the TNFR1 in a standard cell assay (e.g., the standard L929 or standard HeLa IL-8 assays described herein) with an ND₅₀ of ≦100 nM, and at a concentration of ≦10 μM the dAb agonizes the activity of the TNFR1 by ≦5% in the assay.

In other embodiments, the anti-TNFR1 dAb monomer specifically binds TNFR1 with a K_{d} described herein and inhibits lethality in a standard mouse LPS/D-galactosamine-induced septic shock model (i.e., prevents lethality or reduces lethality by at least about 10%, as compared with a suitable control). Preferably, the anti-TNFR1 dAb monomer inhibits lethality by at least about 25%, or by at least about 50%, as compared to a suitable control in a standard mouse LPS/D-galactosamine-induced septic shock model when administered at about 5 mg/kg or more preferably about 1 mg/kg.

In particular embodiments, the anti-TNFR1 dAb monomer or a ligand of the invention that comprises such a dAb monomer, does not substantially agonize TNFR1 (act as an agonist of TNFR1) in a standard cell assay, such as the standard L929 or standard HeLa IL-8 assays described herein (i.e., when present at a concentration of 1 nM, 10 nM, 100 nM, 1 μM, 10 μM, 100 μM, 1000 μM or 5,000 μM, results in no more than about 5% of the TNFR1-mediated activity induced by TNFα (100 pg/ml) in the assay).

In other embodiments, the ligand comprises a domain antibody (dAb) monomer that specifically binds Tumor Necrosis Factor Receptor 1 (TNFR1, p55, CD120a) with a K_{d} of 300 nM to 5 pM, and comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence or a dAb selected from the group consisting of TAR2h-12 (SEQ ID NO:785), TAR2h-13 (SEQ ID NO:786), TAR2h-14 (SEQ ID NO:787), TAR2h-16 (SEQ ID NO:788), TAR2h-17 (SEQ ID NO:789), TAR2h-18 (SEQ ID NO:790), TAR2h-19 (SEQ ID NO:791), TAR2h-20 (SEQ ID NO:792), TAR2h-21 (SEQ ID NO:793), TAR2h-22 (SEQ ID NO:794), TAR2h-23 (SEQ ID NO:795), TAR2h-24 (SEQ ID NO:796), TAR2h-25 (SEQ ID NO:797), TAR2h-26 (SEQ ID NO:798), TAR2h-27 (SEQ ID NO:799), TAR2h-29 (SEQ ID NO:800), TAR2h-30 (SEQ ID NO:801), TAR2h-32 (SEQ ID NO:802), TAR2h-33 (SEQ ID NO:803), TAR2h-10-1 (SEQ ID NO:804), TAR2h-10⁻² (SEQ ID NO:805), TAR2h-10⁻³ (SEQ ID NO:806), TAR2h-10⁻⁴ (SEQ ID NO:807), TAR2h-10-5 (SEQ ID NO:808), TAR2h-10-6 (SEQ ID NO:809), TAR2h-10-7 (SEQ ID NO:810), TAR2h-10⁻⁸ (SEQ ID NO:811), TAR2h-10⁻⁹ (SEQ ID NO:812), TAR2h-10-10 (SEQ ID NO:813), TAR2h-10-11 (SEQ ID NO:814), TAR2h-10-12 (SEQ ID NO:815), TAR2h-10-13 (SEQ ID NO:816), TAR2h-10-14 (SEQ ID NO:817), TAR2h-10-15 (SEQ ID NO:818), TAR2h-10-16 (SEQ ID NO:819), TAR2h-10-17 (SEQ ID NO:820), TAR2h-10-18 (SEQ ID NO:821), TAR2h-10-19 (SEQ ID NO:822), TAR2h-10-20 (SEQ ID NO:823), TAR2h-10-21 (SEQ ID NO:824), TAR2h-10-22 (SEQ ID NO:825), TAR2h-10-27 (SEQ ID NO:826), TAR2h-10-29 (SEQ ID NO:827), TAR2h-10-31 (SEQ ID NO:828), TAR2h-10-35 (SEQ ID NO:829), TAR2h-10-36 (SEQ ID NO:830), TAR2h-10-37 (SEQ ID NO:831), TAR2h-10-38 (SEQ ID NO:832), TAR2h-10-45 (SEQ ID NO:833), TAR2h-10-47 (SEQ ID NO:834), TAR2h-10-48 (SEQ ID NO:835), TAR2h-10-57 (SEQ ID NO:836), TAR2h-10-56 SEQ ID NO:837), TAR2h-10-58 (SEQ ID NO:838), TAR2h-10-66 (SEQ ID NO:839), TAR2h-10-64 (SEQ ID NO:840), TAR2h-10-65 (SEQ ID NO:841), TAR2h-10-68 (SEQ ID NO:842), TAR2h-10-69 (SEQ ID NO:843), TAR2h-10-67 (SEQ ID NO:844), TAR2h-10-61 (SEQ ID NO:845), TAR2h-10-62 (SEQ ID NO:846), TAR2h-10-63 (SEQ ID NO:847), TAR2h-10-60 (SEQ ID NO:848), TAR2h-10-55 (SEQ ID NO:849), TAR2h-10-59 (SEQ ID NO:850), TAR2h-10-70 (SEQ ID NO:851), TAR2h-34 (SEQ ID NO:852), TAR2h-35 (SEQ ID NO:853), TAR2h-36 (SEQ ID NO:854), TAR2h-37 (SEQ ID NO:855), TAR2h-38 (SEQ ID NO:856), TAR2h-39 (SEQ ID NO:857), TAR2h-40 (SEQ ID NO:858), TAR2h-41 (SEQ ID NO:859), TAR2h-42 (SEQ ID NO:860), TAR2h-43 (SEQ ID NO:861), TAR2h-44 (SEQ ID NO:862), TAR2h-45 (SEQ ID NO:863), TAR2h-47 (SEQ ID NO:864), TAR2h-48 (SEQ ID NO:865), TAR2h-50 (SEQ ID NO:866), TAR2h-51 (SEQ ID NO:867), TAR2h-66 (SEQ ID NO:868), TAR2h-67 (SEQ ID NO:869), TAR2h-68 (SEQ ID NO:870), TAR2h-70 (SEQ ID NO:871), TAR2h-71 (SEQ ID NO:872), TAR2h-72 (SEQ ID NO:873), TAR2h-73 (SEQ ID NO:874), TAR2h-74 (SEQ ID NO:875), TAR2h-75 (SEQ ID NO:876), TAR2h-76 (SEQ ID NO:877), TAR2h-77 (SEQ ID NO:878), TAR2h-78 (SEQ ID NO:879), TAR2h-79 (SEQ ID NO:880), TAR2h-15 (SEQ ID NO:881), TAR2h-131-8 (SEQ ID NO:882), TAR2h-131-24 (SEQ ID NO:883), TAR2h-15-8 (SEQ ID NO:884), TAR2h-15-8 (SEQ ID NO:885), TAR2h-15-8-2 (SEQ ID NO:886), TAR2h-1185-23 (SEQ ID NO:887), TAR2h-154-10⁻⁵ (SEQ ID NO:888), TAR2h-14-2 (SEQ ID NO:889), TAR2h-151-8 (SEQ ID NO:890), TAR2h-152-7 (SEQ ID NO:891), TAR2h-35-4 (SEQ ID NO:892), TAR2h-154-7 (SEQ ID NO:893), TAR2h-80 (SEQ ID NO:894), TAR2h-81 (SEQ ID NO:895), TAR2h-82 (SEQ ID NO:896), TAR2h-83 (SEQ ID NO:897), TAR2h-84 (SEQ ID NO:898), TAR2h-85 (SEQ ID NO:899), TAR2h-86 (SEQ ID NO:900), TAR2h-87 (SEQ ID NO:901), TAR2h-88 (SEQ ID NO:902), TAR2h-89 (SEQ ID NO:903), TAR2h-90 (SEQ ID NO:904), TAR2h-91 (SEQ ID NO:905), TAR2h-92 (SEQ ID NO:906), TAR2h-93 (SEQ ID NO:907), TAR2h-94 (SEQ ID NO:908), TAR2h-95 (SEQ ID NO:909), TAR2h-96 (SEQ ID NO:910), TAR2h-97 (SEQ ID NO:911), TAR2h-99 (SEQ ID NO:912), TAR2h-100 (SEQ ID NO:913), TAR2h-101 (SEQ ID NO:914), TAR2h-102 (SEQ ID NO:915), TAR2h-103 (SEQ ID NO:916), TAR2h-104 (SEQ ID NO:917), TAR2h-105 (SEQ ID NO:918), TAR2h-106 (SEQ ID NO:919), TAR2h-107 (SEQ ID NO:920), TAR2h-108 (SEQ ID NO:921), TAR2h-109 (SEQ ID NO:922), TAR2h-110 (SEQ ID NO:923), TAR2h-111 (SEQ ID NO:924), TAR2h-112 (SEQ ID NO:925), TAR2h-113 (SEQ ID NO:926), TAR2h-114 (SEQ ID NO:927), TAR2h-115 (SEQ ID NO:928), TAR2h-116 (SEQ ID NO:929), TAR2h-117 (SEQ ID NO:930), TAR2h-118 (SEQ ID NO:931), TAR2h-119 (SEQ ID NO:932), TAR2h-120 (SEQ ID NO:933), TAR2h-121 (SEQ ID NO:934), TAR2h-122 (SEQ ID NO:935), TAR2h-123 (SEQ ID NO:936), TAR2h-124 (SEQ ID NO:937), TAR2h-125 (SEQ ID NO:938), TAR2h-126 (SEQ ID NO:939), TAR2h-127 (SEQ ID NO:940), TAR2h-128 (SEQ ID NO:941), TAR2h-129 (SEQ ID NO:942), TAR2h-130 (SEQ ID NO:943), TAR2h-131 (SEQ ID NO:944), TAR2h-132 (SEQ ID NO:945), TAR2h-133 (SEQ ID NO:946), TAR2h-151 (SEQ ID NO:947), TAR2h-152 (SEQ ID NO:948), TAR2h-153 (SEQ ID NO:949), TAR2h-154 (SEQ ID NO:950), TAR2h-159 (SEQ ID NO:951), TAR2h-165 (SEQ ID NO:952), TAR2h-166 (SEQ ID NO:953), TAR2h-168 (SEQ ID NO:954), TAR2h-171 (SEQ ID NO:955), TAR2h-172 (SEQ ID NO:956), TAR2h-173 (SEQ ID NO:957), TAR2h-174 (SEQ ID NO:958), TAR2h-176 (SEQ ID NO:959), TAR2h-178 (SEQ ID NO:960), TAR2h-201 (SEQ ID NO:961), TAR2h-202 (SEQ ID NO:962), TAR2h-203 (SEQ ID NO:963), TAR2h-204 (SEQ ID NO:964), TAR2h-185-25 (SEQ ID NO:965), TAR2h-154-10 SEQ ID NO:966), TAR2h-205 (SEQ ID NO:967), TAR2h-10 (SEQ ID NO:968), TAR2h-5 (SEQ ID NO:969), TAR2h-5d1 (SEQ ID NO:970), TAR2h-5d2 (SEQ ID NO:971), TAR2h-5d3 (SEQ ID NO:972), TAR2h-5d4 (SEQ ID NO:973), TAR2h-5d5 (SEQ ID NO:974), TAR2h-5d6 (SEQ ID NO:975), TAR2h-5d7 (SEQ ID NO:976), TAR2h-5d8 (SEQ ID NO:977), TAR2h-5d9 (SEQ ID NO:978), TAR2h-5d10 (SEQ ID NO:979), TAR2h-5d11 (SEQ ID NO:980), TAR2h-5d12 (SEQ ID NO:981), and TAR2h-5d13 (SEQ ID NO:982).

In other embodiments, the ligand comprises a domain antibody (dAb) monomer that specifically binds Tumor Necrosis Factor Receptor 1 (TNFR1, p55, CD120a) with a K_{d} of 300 nM to 5 pM, and competes for binding to human TNFR1 with a dAb selected from the group consisting of TAR2h-12 (SEQ ID NO:785), TAR2h-13 (SEQ ID NO:786), TAR2h-14 (SEQ ID NO:787), TAR2h-16 (SEQ ID NO:788), TAR2h-17 (SEQ ID NO:789), TAR2h-18 (SEQ ID NO:790), TAR2h-19 (SEQ ID NO:791), TAR2h-20 (SEQ ID NO:792), TAR2h-21 (SEQ ID NO:793), TAR2h-22 (SEQ ID NO:794), TAR2h-23 (SEQ ID NO:795), TAR2h-24 (SEQ ID NO:796), TAR2h-25 (SEQ ID NO:797), TAR2h-26 (SEQ ID NO:798), TAR2h-27 (SEQ ID NO:799), TAR2h-29 (SEQ ID NO:800), TAR2h-30 (SEQ ID NO:801), TAR2h-32 (SEQ ID NO:802), TAR2h-33 (SEQ ID NO:803), TAR2h-10-1 (SEQ ID NO:804), TAR2h-10⁻² (SEQ ID NO:805), TAR2h-10-3 (SEQ ID NO:806), TAR2h-10⁻⁴ (SEQ ID NO:807), TAR2h-10⁻⁵ (SEQ ID NO:808), TAR2h-10⁻⁶ (SEQ ID NO:809), TAR2h-10⁻⁷ (SEQ ID NO:810), TAR2h-10-8 (SEQ ID NO:811), TAR2h-10⁻⁹ (SEQ ID NO:812), TAR2h-10-10 (SEQ ID NO:813), TAR2h-10-11 (SEQ ID NO:814), TAR2h-10-12 (SEQ ID NO:815), TAR2h-10-13 (SEQ ID NO:816), TAR2h-10-14 (SEQ ID NO:817), TAR2h-10-15 (SEQ ID NO:818), TAR2h-10-16 (SEQ ID NO:819), TAR2h-10-17 (SEQ ID NO:820), TAR2h-10-18 (SEQ ID NO:821), TAR2h-10-19 (SEQ ID NO:822), TAR2h-10-20 (SEQ ID NO:823), TAR2h-10-21 (SEQ ID NO:824), TAR2h-10-22 (SEQ ID NO:825), TAR2h-10-27 (SEQ ID NO:826), TAR2h-10-29 (SEQ ID NO:827), TAR2h-10-31 (SEQ ID NO:828), TAR2h-10-35 (SEQ ID NO:829), TAR2h-10-36 (SEQ ID NO:830), TAR2h-10-37 (SEQ ID NO:831), TAR2h-10-38 (SEQ ID NO:832), TAR2h-10-45 (SEQ ID NO:833), TAR2h-10-47 (SEQ ID NO:834), TAR2h-10-48 (SEQ ID NO:835), TAR2h-10-57 (SEQ ID NO:836), TAR2h-10-56 SEQ ID NO:837), TAR2h-10-58 (SEQ ID NO:838), TAR2h-10-66 (SEQ ID NO:839), TAR2h-10-64 (SEQ ID NO:840), TAR2h-10-65 (SEQ ID NO:841), TAR2h-10-68 (SEQ ID NO:842), TAR2h-10-69 (SEQ ID NO:843), TAR2h-10-67 (SEQ ID NO:844), TAR2h-10-61 (SEQ ID NO:845), TAR2h-10-62 (SEQ ID NO:846), TAR2h-10-63 (SEQ ID NO:847), TAR2h-10-60 (SEQ ID NO:848), TAR2h-10-55 (SEQ ID NO:849), TAR2h-10-59 (SEQ ID NO:850), TAR2h-10-70 (SEQ ID NO:851), TAR2h-34 (SEQ ID NO:852), TAR2h-35 (SEQ ID NO:853), TAR2h-36 (SEQ ID NO:854), TAR2h-37 (SEQ ID NO:855), TAR2h-38 (SEQ ID NO:856), TAR2h-39 (SEQ ID NO:857), TAR2h-40 (SEQ ID NO:858), TAR2h-41 (SEQ ID NO:859), TAR2h-42 (SEQ ID NO:860), TAR2h-43 (SEQ ID NO:861), TAR2h-44 (SEQ ID NO:862), TAR2h-45 (SEQ ID NO:863), TAR2h-47 (SEQ ID NO:864), TAR2h-48 (SEQ ID NO:865), TAR2h-50 (SEQ ID NO:866), TAR2h-51 (SEQ ID NO:867), TAR2h-66 (SEQ ID NO:868), TAR2h-67 (SEQ ID NO:869), TAR2h-68 (SEQ ID NO:870), TAR2h-70 (SEQ ID NO:871), TAR2h-71 (SEQ ID NO:872), TAR2h-72 (SEQ ID NO:873), TAR2h-73 (SEQ ID NO:874), TAR2h-74 (SEQ ID NO:875), TAR2h-75 (SEQ ID NO:876), TAR2h-76 (SEQ ID NO:877), TAR2h-77 (SEQ ID NO:878), TAR2h-78 (SEQ ID NO:879), TAR2h-79 (SEQ ID NO:880), TAR2h-15 (SEQ ID NO:881), TAR2h-131-8 (SEQ ID NO:882), TAR2h-131-24 (SEQ ID NO:883), TAR2h-15-8 (SEQ ID NO:884), TAR2h-15-8-1 (SEQ ID NO:885), TAR2h-15-8-2 (SEQ ID NO:886), TAR2h-185-23 (SEQ ID NO:887), TAR2h-154-10-5 (SEQ ID NO:888), TAR2h-14-2 (SEQ ID NO:889), TAR2h-151-8 (SEQ ID NO:890), TAR2h-152-7 (SEQ ID NO:891), TAR2h-35-4 (SEQ ID NO:892), TAR2h-154-7 (SEQ ID NO:893), TAR2h-80 (SEQ ID NO:894), TAR2h-81 (SEQ ID NO:895), TAR2h-82 (SEQ ID NO:896), TAR2h-83 (SEQ ID NO:897), TAR2h-84 (SEQ ID NO:898), TAR2h-85 (SEQ ID NO:899), TAR2h-86 (SEQ ID NO:900), TAR2h-87 (SEQ ID NO:901), TAR2h-88 (SEQ ID NO:902), TAR2h-89 (SEQ ID NO:903), TAR2h-90 (SEQ ID NO:904), TAR2h-91 (SEQ ID NO:905), TAR2h-92 (SEQ ID NO:906), TAR2h-93 (SEQ ID NO:907), TAR2h-94 (SEQ ID NO:908), TAR2h-95 (SEQ ID NO:909), TAR2h-96 (SEQ ID NO:910), TAR2h-97 (SEQ ID NO:911), TAR2h-99 (SEQ ID NO:912), TAR2h-100 (SEQ ID NO:913), TAR2h-101 (SEQ ID NO:914), TAR2h-102 (SEQ ID NO:915), TAR2h-103 (SEQ ID NO:916), TAR2h-104 (SEQ ID NO:917), TAR2h-105 (SEQ ID NO:918), TAR2h-106 (SEQ ID NO:919), TAR2h-107 (SEQ ID NO:920), TAR2h-108 (SEQ ID NO:921), TAR2h-109 (SEQ ID NO:922), TAR2h-110 (SEQ ID NO:923), TAR2h-111 (SEQ ID NO:924), TAR2h-112 (SEQ ID NO:925), TAR2h-113 (SEQ ID NO:926), TAR2h-114 (SEQ ID NO:927), TAR2h-115 (SEQ ID NO:928), TAR2h-116 (SEQ ID NO:929), TAR2h-117 (SEQ ID NO:930), TAR2h-118 (SEQ ID NO:931), TAR2h-119 (SEQ ID NO:932), TAR2h-120 (SEQ ID NO:933), TAR2h-121 (SEQ ID NO:934), TAR2h-122 (SEQ ID NO:935), TAR2h-123 (SEQ ID NO:936), TAR2h-124 (SEQ ID NO:937), TAR2h-125 (SEQ ID NO:938), TAR2h-126 (SEQ ID NO:939), TAR2h-127 (SEQ ID NO:940), TAR2h-128 (SEQ ID NO:941), TAR2h-129 (SEQ ID NO:942), TAR2h-130 (SEQ ID NO:943), TAR2h-131 (SEQ ID NO:944), TAR2h-132 (SEQ ID NO:945), TAR2h-133 (SEQ ID NO:946), TAR2h-151 (SEQ ID NO:947), TAR2h-152 (SEQ ID NO:948), TAR2h-153 (SEQ ID NO:949), TAR2h-154 (SEQ ID NO:950), TAR2h-159 (SEQ ID NO:951), TAR2h-165 (SEQ ID NO:952), TAR2h-166 (SEQ ID NO:953), TAR2h-168 (SEQ ID NO:954), TAR2h-171 (SEQ ID NO:955), TAR2h-172 (SEQ ID NO:956), TAR2h-173 (SEQ ID NO:957), TAR2h-174 (SEQ ID NO:958), TAR2h-176 (SEQ ID NO:959), TAR2h-178 (SEQ ID NO:960), TAR2h-201 (SEQ ID NO:961), TAR2h-202 (SEQ ID NO:962), TAR2h-203 (SEQ ID NO:963), TAR2h-204 (SEQ ID NO:964), TAR2h-185-25 (SEQ ID NO:965), TAR2h-154-10 SEQ ID NO:966), TAR2h-205 (SEQ ID NO:967), TAR2h-10 (SEQ ID NO:968), TAR2h-5 (SEQ ID NO:969), TAR2h-5d1 (SEQ ID NO:970), TAR2h-5d2 (SEQ ID NO:971), TAR2h-5d3 (SEQ ID NO:972), TAR2h-5d4 (SEQ ID NO:973), TAR2h-5d5 (SEQ ID NO:974), TAR2h-5d6 (SEQ ID NO:975), TAR2h-5d7 (SEQ ID NO:976), TAR2h-5d8 (SEQ ID NO:977), TAR2h-5d9 (SEQ ID NO:978), TAR2h-5d10 (SEQ ID NO:979), TAR2h-5d 11 (SEQ ID NO:980), TAR2h-5d12 (SEQ ID NO:981), and TAR2h-5d13 (SEQ ID NO:982).

In other embodiments, the ligand comprises a domain antibody (dAb) monomer that specifically binds Tumor Necrosis Factor Receptor 1 (TNFR1, p55, CD120a) with a K_{d} of 300 nM to 5 pM, and comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence or a dAb selected from the group consisting of TAR2m-14 (SEQ ID NO:983), TAR2m-15 (SEQ ID NO:984), TAR2m-19 (SEQ ID NO:985), TAR2m-20 (SEQ ID NO:986), TAR2m-21 (SEQ ID NO:987), TAR2m-24 (SEQ ID NO:988), TAR2m-21-23 (SEQ ID NO:989), TAR2m-21-07 (SEQ ID NO:990), TAR2m-21-43 (SEQ ID NO:991), TAR2m-21-48 (SEQ ID NO:992), TAR2m-21-10 (SEQ ID NO:993), TAR2m-21-06 (SEQ ID NO:994), and TAR2m-21-17 (SEQ ID NO:995).

In some embodiments, the ligand comprises a dAb monomer that binds TNFR1 and competes with any of the dAbs disclosed herein for binding to TNFR1 (e.g., mouse and/or human TNFR1).

Protease Resistant dAbs

The invention also relates to dAb monomers that are resistant to protease (e.g., serine protease, cysteine protease, matrix metalloprotease, pepsin, trypsin, elastase, chymotrypsin, carboxypeptidase, cathepsin (e.g., cathepsin G), proteinase 3) degradation and to ligands that comprise a protease resistant dAb. Proteases (e.g., a serine protease, cysteine protease, matrix metalloprotease) function in the normal turn over and metabolism of proteins. However, in certain physiological states, such as inflammatory states (e.g., COPD) and cancer, the amount of proteases present in a tissue, organ or animal (e.g., in the lung, in or adjacent to a tumor) can increase. This increase in proteases can result in accelerated degradation and inactivation of endogenous proteins and of therapeutic peptides, polypeptides and proteins that are administered. In fact, some agents that have potential for in vivo use (e.g., use in treating, diagnosing or preventing disease) have only limited efficacy because they are rapidly degraded and inactivated by proteases.

The invention relates to a dAb or a ligand comprising a dAb that is resistant to protease degradation. The protease resistant dAbs of the invention provide several advantages. For example, a protease resistant dAb can be administered to a subject and remain active in vivo longer than protease sensitive agents. Accordingly, protease resistant dAbs will remain functional for a period of time that is sufficient to produce biological effects.

A dAb that is resistant to protease degradation is not substantially degraded by a protease when incubated with the protease under conditions suitable for protease activity for at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, at least about 36 hours, or at least about 48 hours. A dAb is not substantially degraded when no more than about 25%, no more than about 20%, no more than about 15%, no more than about 14%, no more than about 13%, no more than about 12%, no more than about 11%, no more than about 10%, no more than about 9%, no more than about 8%, no more than about 7% no more than about 6%, no more than about 5%, no more than about 4%, no more than about 3%, no more than about 2%, no more than about 1%, or substantially none of the protein is degraded by protease after incubation with the protease for at least about 2 hours. Protein degradation can be assessed using any suitable method, for example, by SDS-PAGE as described herein.

Protease resistance can be assessed using any suitable method. For example, a protease can be added to a solution of dAb in a suitable buffer (e.g., PBS) to produce a dAb/protease solution, such as a solution of at least about 0.01% (w/w) protease, about 0.01% to about 5% (w/w) protease, about 0.05% to about 5% (w/w) protease, about 0.1% to about 5% (w/w) protease, about 0.5% to about 5% (w/w) protease, about 1% to about 5% (w/w) protease, at least about 0.01% (w/w) protease, at least about 0.02% (w/w) protease, at least about 0.03% (w/w) protease, at least about 0.04% (w/w) protease, at least about 0.05% (w/w) protease, at least about 0.06% (w/w) protease, at least about 0.07% (w/w) protease, at least about 0.08% (w/w) protease, at least about 0.09% (w/w) protease, at least about 0.1% (w/w) protease, at least about 0.2% (w/w) protease, at least about 0.3% (w/w) protease, at least about 0.4% (w/w) protease, at least about 0.5% (w/w) protease, at least about 0.6% (w/w) protease, at least about 0.7% (w/w) protease, at least about 0.8% (w/w) protease, at least about 0.9% (w/w) protease, at least about 1% (w/w) protease, at least about 2% (w/w) protease, at least about 3% (w/w) protease, at least about 4% (w/w) protease, or about 5% (w/w) protease. The dAb/protease mixture can be incubated at a suitable temperature for protease activity (e.g., at 37° C.) and samples can be taken at time intervals (e.g., at 1 hour, 2 hours, 3 hours, etc.) and the protease reaction stopped. The samples can then be analyzed for protein degradation using any suitable method, such as SDS-PAGE analysis. The results can be used to establish a time course of degradation.

In particular embodiments, the protease resistant dAb is resistant to degradation by elastase. For example, the elastase resistant dAb is not substantially degraded when incubated at 37° C. in a 0.04% (w/w) solution of elastase for a period of at least about 2 hours. Preferably, the elastase resistant dAb is not substantially degraded when incubated at 37° C. in a 0.04% (w/w) solution of elastase for a period of at least about 12 hours. More preferably, the elastase resistant dAb is not substantially degraded when incubated at 37° C. in a 0.04% (w/w) solution of elastase for a period of at least about 24 hours, at least about 36 hours, or at least about 48 hours.

In particular embodiments, the protease resistant dAb is resistant to degradation by trypsin. For example, the trypsin resistant dAb is not substantially degraded when incubated at 37° C. in a 0.04% (w/w) solution of trypsin for a period of at least about 2 hours. Preferably, the trypsin resistant dAb is not substantially degraded when incubated at 37° C. in a 0.04% (w/w) solution of trypsin for a period of at least about 3 hours. More preferably, the trypsin resistant dAb is not substantially degraded when incubated at 37° C. in a 0.04% (w/w) solution of trypsin for a period of at least about 4 hours, at least about 5 hours, at least about 6 hours, at least about 7 hours, at least about 8 hours, at least about 9 hours, at least about 10 hours, at least about 11 hours, or at least about 12 hours.

In certain embodiments, the invention does not include TAR1-5-19 disclosed in WO 2004/081026.

Preferably, the protease resistant dAb is a light chain variable domain. For example, the protease resistant dAb can be a Vκ or a Vλ.

Protease resistance of dAbs can correlate with the melting temperature (Tm) of the dAbs. Generally, a higher melting temperature correlates with protease resistance. In some embodiments, the protease resistant dAb has a Tm between about 40° C. and about 95° C., about 40° C. and about 85° C., about 40° C. and about 80° C., about 45° C. and about 95° C., about 45° C. and about 85° C., 45° C. and about 80° C., at least about 40° C., at least about 45° C., at least about 50° C., at least about 55° C., at least about 60° C., at least about 65° C., at least about 70° C., at least about 75° C., at least about 80° C., at least about 85° C., at least about 90° C., or at least about 95° C.

The protease resistant dAb can have binding specificity for any desired target, such as human or animal proteins, including cytokines, growth factors, cytokine receptors, growth factor receptors, enzymes (e.g., proteases), co-factors for enzymes and DNA binding proteins, lipids and carbohydrates. Suitable targets, including cytokines, growth factors, cytokine receptors, growth factor receptors and other proteins include but are not limited to: ApoE, Apo-SAA, BDNF, Cardiotrophin-1, CEA, CD40, CD40 Ligand, CD56, CD38, CD138, EGF, EGF receptor, ENA-78, Eotaxin, Eotaxin-2, Exodus-2, FAPα, FGF-acidic, FGF-basic, fibroblast growth factor-10, FLT3 ligand, Fractalkine (CX3C), GDNF, G-CSF, GM-CSF, GF-β1, human serum albumin, insulin, IFN-γ, IGF-I, IGF-II, IL-1α, IL-1β, IL-1 receptor, IL-1 receptor type 1, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF A, VEGF B, VEGF C, VEGF D, VEGF receptor 1, VEGF receptor 2, VEGF receptor 3, GCP-2, GRO/MGSA, GRO-β, GRO-γ, HCC1, 1-309, HER 1, HER 2, HER 3, HER 4, serum albumin, vWF, amyloid proteins (e.g., amyloid alpha), MMP12, PDK1, IgE, and other targets disclosed herein. It will be appreciated that this list is by no means exhaustive.

In some embodiments, the protease resistant dAbs binds a target in pulmonary tissue, such as a target selected from the group consisting of TNFR1, IL-1, IL-1R, IL-4, IL-4R, IL-5, IL-6, IL-6R, IL-8, IL-8R, IL-9, IL-9R, IL-10, IL-12 IL-12R, IL-13, IL-13Rα1, IL-13Ra2, IL-15, IL-15R, IL-16, IL-17R, IL-17, IL-18, IL-18R, IL-23 IL-23R, IL-25, CD2, CD4, CD11a, CD23, CD25, CD27, CD28, CD30, CD40, CD40L, CD56, CD138, ALK5, EGFR, FcER1, TGFb, CCL2, CCL18, CEA, CR8, CTGF, CXCL12 (SDF-1), chymase, FGF, Furin, Endothelin-1, Eotaxins (e.g., Eotaxin, Eotaxin-2, Eotaxin-3), GM-CSF, ICAM-1, ICOS, IgE, IFNa, I-309, integrins, L-selectin, MIF, MIP4, MDC, MCP-1, MMPs, neutrophil elastase, osteopontin, OX-40, PARC, PD-1, RANTES, SCF, SDF-1, siglec8, TARC, TGFb, Thrombin, Tim-1, TNF, TRANCE, Tryptase, VEGF, VLA-4, VCAM, α4β7, CCR2, CCR3, CCR4, CCR5, CCR7, CCR8, alphavbeta6, alphavbeta8, cMET, CD8, vWF, amyloid proteins (e.g., amyloid alpha), MMP12, PDK1, and IgE.

The protease resistant dAbs of the invention can be administered in vivo and will remain functional longer than compounds that are not similarly resistant to protease degradation. A dAb of the invention that is resistant to protease degradation can be used for treating an inflammatory disease (e.g., by local delivery to the lung by pulmonary administration, e.g., by intranasal administration, e.g., by inhalation). For example, by administering to a subject in need thereof a therapeutically effective amount of a dAb monomer that is resistant to protease degradation. The invention also relates to a dAb monomer that is resistant to protease degradation for use in therapy, diagnosis and/or prophylaxis, and to the use of such a dAb monomer of the invention for the manufacture of a medicament for treating a disease described herein (e.g., and inflammatory disease, arthritis, a respiratory disease).

In particular embodiments, the protease resistant dAb monomer can be used for treating an inflammatory disease, arthritis, or a respiratory disease via pulmonary administration. The protease resistant dAb monomer can also be used in the manufacture of a medicament for the treatment of an inflammatory disease, arthritis, or a respiratory disease wherein the dAb monomer is administered via pulmonary administration. Elastase and trypsin are the most common proteases found in the lung. Preferably, protease resistant dAbs for pulmonary administration are elastase resistant, trypsin resistant, or elastase resistant and trypsin resistant.

In particular embodiments, the protease resistant dAb monomer (e.g., elastase resistant dAb monomer) binds IL-1R1 and inhibits binding of IL-1 (e.g., IL-1α and/or IL-1β) to the receptor but does not inhibit binding of IL-1ra to IL-1R1, and to ligands comprising such dAb monomers. Such dAb monomers are useful as therapeutic agents for treating inflammation, disease or other condition mediated in whole or in part by biological functions induced by binding of IL-1 to IL-1R1 (e.g., local or systemic inflammation, elaboration of inflammatory mediators (e.g., IL-6, Il-8, TNF), fever, activation immune cells (e.g., lymphocytes, neutrophils), anorexia, hypotension, leucopenia, thrombocytopenia.) The protease resistant dAb monomers can bind IL-1R1 and inhibit IL-1R1 function without interfering with endogenous IL-1R1 inhibitory pathways, such as binding of endogenous IL-1ra to endogenous IL-1R1. Accordingly, such a dAb monomer can be administered to a subject to complement the endogenous regulatory pathways that inhibit the activity of IL-1R1 or IL-1 in vivo. In addition, protease resistant dAb monomers that bind and IL-1R1 do not inhibit binding of IL-1ra to IL-1R1 provide advantages for use as diagnostic agents, because they can be used to bind and detect, quantify or measure IL-1R1 in a sample and will not compete with IL-1ra in the sample for binding to IL-1R1. Accordingly, an accurate determination of whether or how much IL-1R1 is in the sample can be made.

Protease resistant dAb monomers (e.g., elastase resistant dAb monomers) that bind IL-1R1 and inhibit binding of IL-1 (e.g., IL-1α and/or IL-1β) to the receptor but do not inhibit binding of IL-1ra to IL-1R1 are also useful research tools. For example, such a dAb monomer can be used to identify agents (e.g., other dAbs, small organic molecules) that bind IL-1R1 and but do not inhibit binding of IL-1ra to IL-1R1. In one illustrative example, an agent or collection of agents to be tested for the ability to inhibit binding of IL-1 to IL-1R1 are assayed in a competitive IL-1R1 receptor binding assay, such as the receptor binding assay described herein. Agents that inhibit binding of IL-1 to IL-1R1 in such an assay can then be studied in a similar competitive IL-1R1 receptor binding assay to see if they compete with a dAb monomer that binds IL-1R1 but does not inhibit binding of IL-1ra to IL-1R1. Competitive binding in such an assay indicates that the agent binds IL-1R1 and inhibits binding of IL-1 to the receptor but does not inhibit binding of IL-1ra to the receptor.

In some embodiments, the protease resistant dAb binds IL-1R1 and competes with any of the dAbs disclosed herein for binding to IL-1R1 (e.g., human IL-1R1). In some embodiments the dAb is resistant to at least elastase and/or trypsin.

In other embodiments, the protease resistant dAb competes for binding to IL-1R1 with an anti-IL-1R1 dAb, wherein the anti-IL-1R1 dAb comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence or a dAb selected from the group consisting of SEQ ID NO:1 through SEQ ID NO:349.

In other embodiments, the protease resistant dAb competes for binding to IL-1R1 with an anti-IL-1R1 dAb, wherein the anti-IL-1R1 dAb comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence or a dAb selected from the group consisting of SEQ ID NO:1 or SEQ ID NO:2.

In other embodiments, the protease resistant dAb competes for binding to IL-1R1 with an anti-IL-1R1 dAb, wherein the anti-IL-1R1 dAb comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence or a dAb selected from the group consisting of SEQ ID NO:3 through SEQ ID NO:7.

In other embodiments, the protease resistant dAb competes for binding to IL-1R1 with an anti-IL-1R1 dAb, wherein the anti-IL-1R1 dAb comprises the amino acid sequence DOM4-130-54 (SEQ ID NO:7) or an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to DOM4-130-54 (SEQ ID NO:7).

### Ligand Formats

Ligands and dAb monomers can be formatted as mono or multispecific antibodies or antibody fragments or into mono or multispecific non-antibody structures. Suitable formats include, any suitable polypeptide structure in which an antibody variable domain or one or more of the CDRs thereof can be incorporated so as to confer binding specificity for antigen on the structure. A variety of suitable antibody formats are known in the art, such as, IgG-like formats, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., V_{H}, V_{L}, V_{HH}), a dAb, and modified versions of any of the foregoing (e.g., modified by the covalent attachment of polyalkylene glycol (e.g., polyethylene glycol, polypropylene glycol, polybutylene glycol) or other suitable polymer). See, PCT/GB03/002804, filed Jun. 30, 2003, which designated the United States, (WO 2004/081026) regarding PEGylated single variable domains and dAbs, suitable methods for preparing same, increased in vivo half life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

The ligand can be formatted as a dimer, trimer or polymer of a desired dAb monomer, for example using a suitable linker such as (Gly₄Ser)ₙ, where n=from 1 to 8, e.g., 2, 3, 4, 5, 6 or 7. If desired, ligands, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides.

Ligands and dAb monomers can also be combined and/or formatted into non-antibody multi-ligand structures to form multivalent complexes, which bind target molecules, thereby providing superior avidity. For example natural bacterial receptors such as SpA can been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides. Protein scaffolds may be combined; for example, CDRs may be grafted on to a CTLA4 scaffold and used together with immunoglobulin V_{H} or V_{L} domains to form a ligand. Likewise, fibronectin, lipocallin and other scaffolds may be combined.

A variety of suitable methods for preparing any desired format are known in the art. For example, antibody chains and formats (e.g., IgG-like formats, chimeric antibodies, humanized antibodies, human antibodies, single chain antibodies, bispecific antibodies, antibody heavy chains, antibody light chains, homodimers and heterodimers of antibody heavy chains and/or light chains) can be prepared by expression of suitable expression constructs and/or culture of suitable cells (e.g., hybridomas, heterohybridomas, recombinant host cells containing recombinant constructs encoding the format). Further, formats such as antigen-binding fragments of antibodies or antibody chains (e.g., a Fv fragment (e.g., single chain Fv (scFv), a disulfide bonded Fv), a Fab fragment, a Fab′ fragment, a F(ab′)₂ fragment), can be prepared by expression of suitable expression constructs or by enzymatic digestion of antibodies, for example using papain or pepsin.

The ligand can be formatted as a dual specific ligand or a multispecific ligand, for example as described in WO 03/002609, the entire teachings of which are incorporated herein by reference. The dual specific ligands comprise immunoglobulin single variable domains that have different binding specificities. Such dual specific ligands can comprise combinations of heavy and light chain domains. For example, the dual specific ligand may comprise a V_{H} domain and a V_{L} domain, which may be linked together in the form of an scFv (e.g., using a suitable linker such as Gly₄Ser), or formatted into a bispecific antibody or antigen-binding fragment thereof (e.g., F(ab′)₂ fragment). The dual specific ligands do not comprise complementary V_{H}/V_{L} pairs which form a conventional two chain antibody antigen-binding site that binds antigen or epitope co-operatively. Instead, the dual format ligands comprise a V_{H}/V_{L} complementary pair, wherein the V domains have different binding specificities.

In addition, the dual specific ligands may comprise one or more C_{H} or C_{L} domains if desired. A hinge region domain may also be included if desired. Such combinations of domains may, for example, mimic natural antibodies, such as IgG or IgM, or fragments thereof, such as Fv, scFv, Fab or F(ab′)₂ molecules. Other structures, such as a single arm of an IgG molecule comprising V_{H}, V_{L}, C_{H}1 and C_{L} domains, are envisaged. Preferably, the dual specific ligand of the invention comprises only two variable domains although several such ligands may be incorporated together into the same protein, for example two such ligands can be incorporated into an IgG or a multimeric immunoglobulin, such as IgM. Alternatively, in another embodiment a plurality of dual specific ligands are combined to form a multimer. For example, two different dual specific ligands are combined to create a tetra-specific molecule. It will be appreciated by one skilled in the art that the light and heavy variable regions of a dual-specific ligand produced according to the method of the present invention may be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

The multispecific ligand possesses more than one epitope binding specificity. Generally, the multi-specific ligand comprises two or more epitope binding domains, such dAbs or non-antibody protein domain comprising a binding site for an epitope, e.g., an affibody, an SpA domain, an LDL receptor class A domain, an EGF domain, an avimer. Multispecific ligands can be formatted further as described herein.

In some embodiments, the ligand is an IgG-like format. Such formats have the conventional four chain structure of an IgG molecule (2 heavy chains and two light chains), in which one or more of the variable regions (V_{H} and or V_{L}) have been replaced with a dAb or single variable domain of a desired specificity. Preferably, each of the variable regions (2 V_{H} regions and 2 V_{L} regions) is replaced with a dAb or single variable domain. The dAb(s) or single variable domain(s) that are included in an IgG-like format can have the same specificity or different specificities. In some embodiments, the IgG-like format is tetravalent and can have one, two, three or four specificities. For example, the IgG-like format can be monospecific and comprises 4 dAbs that have the same specificity; bispecific and comprises 3 dAbs that have the same specificity and another dAb that has a different specificity; bispecific and comprise two dAbs that have the same specificity and two dAbs that have a common but different specificity; trispecific and comprises first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)₂, Fab′, Fv, scFv) can be prepared. Preferably, the IgG-like formats or antigen-binding fragments thereof do not crosslink TNFR1.

### Half-Life Extended Formats

The ligand, such as a dAb monomers, can be formatted to extend its in vivo serum half life. Increased in vivo half-life is useful in in vivo applications of immunoglobulins, especially antibodies and most especially antibody fragments of small size such as dAbs. Such fragments (Fvs, disulphide bonded Fvs, Fabs, scFvs, dAbs) are rapidly cleared from the body, which can limit clinical applications.

Small ligands, such as a dAb monomer, can be formatted as a larger antigen-binding fragment of an antibody or as an antibody (e.g., formatted as a Fab, Fab′, F(ab)₂, F(ab′)₂, IgG, scFv). A ligand (e.g., dAb monomer) can be formatted as a larger antigen-binding fragment of an antibody or as an antibody (e.g., formatted as a Fab, Fab′, F(ab)₂, F(ab′)₂, IgG, scFv) that has a larger hydrodynamic size. Ligands can also be formatted to have a larger hydrodynamic size, for example, by attachment of a polyalkyleneglycol group (e.g., polyethyleneglycol (PEG) group, polypropylene glycol, polybutylene glycol), serum albumin, transferrin, transferrin receptor or at least the transferrin-binding portion thereof, an antibody Fc region, or by conjugation to an antibody domain. In some embodiments, the ligand (e.g., dAb monomer) is PEGylated. Preferably the PEGylated ligand (e.g., dAb monomer) binds IL-1R1 with substantially the same affinity as the same ligand that is not PEGylated. For example, the ligand can be a PEGylated dAb monomer that binds IL-1R1, wherein the PEGylated dAb monomer binds IL-1R1 with an affinity that differs from the affinity of dAb in unPEGylated form by no more than a factor of about 1000, preferably no more than a factor of about 100, more preferably no more than a factor of about 10, or with substantially unchanged affinity relative to the unPEGylated form. See, PCT/GB03/002804, filed Jun. 30, 2003, which designated the United States, (WO 2004/081026) regarding PEGylation of single variable domains and dAbs, suitable methods for preparing same, increased in vivo half life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

Hydrodynamic size of the ligands (e.g., dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g., the size of a PEG moiety attached to a dAb monomer), can be varied depending on the desired application. For example, where ligand is intended to leave the circulation and enter into peripheral tissues, it is desirable to keep the hydrodynamic size of the ligand low to facilitate extravazation from the blood stream. Alternatively, where it is desired to have the ligand remain in the systemic circulation for a longer period of time the size of the ligand can be increased, for example by formatting as and Ig-like protein or by addition of a 30 to 60 kDa PEG moiety (e.g., linear or branched PEG 30 to 40 kDa PEG, such as addition of two 20 kDa PEG moieties.)

The hydrodynamic size of a ligand (e.g., dAb monomer) and its serum half-life can also be increased by conjugating or linking the ligand to a binding domain (e.g., antibody or antibody fragment) that binds an antigen or epitope that increases half-life in vivo, as described herein. For example, the ligand (e.g., dAb monomer) can be conjugated or linked to an anti-serum albumin or anti-neonatal Fc receptor antibody or antibody fragment, e.g. an anti-SA or anti-neonatal Fc receptor dAb, Fab, Fab′ or scFv, or to an anti-SA affibody or anti-neonatal Fc receptor affibody.

Examples of suitable albumin, albumin fragments or albumin variants for use in a ligand according to the invention are described in WO 2005/077042A2, which is incorporated herein by reference in its entirety. In particular, the following albumin, albumin fragments or albumin variants can be used in the present invention:
SEQ ID NO:1 (as disclosed in WO 2005/077042A2, this sequence being explicitly incorporated into the present disclosure by reference);
Albumin fragment or variant comprising or consisting of amino acids 1-387 of SEQ ID NO:1 in WO 2005/077042A2;
Albumin, or fragment or variant thereof, comprising an amino acid sequence selected from the group consisting of: (a) amino acids 54 to 61 of SEQ ID NO:1 in WO 2005/077042A2; (b) amino acids 76 to 89 of SEQ ID NO:1 in WO 2005/077042A2; (c) amino acids 92 to 100 of SEQ ID NO:1 in WO 2005/077042A2; (d) amino acids 170 to 176 of SEQ ID NO:1 in WO 2005/077042A2; (e) amino acids 247 to 252 of SEQ ID NO:1 in WO 2005/077042A2; (f) amino acids 266 to 277 of SEQ ID NO:1 in WO 2005/077042A2; (g) amino acids 280 to 288 of SEQ ID NO:1 in WO 2005/077042A2; (h) amino acids 362 to 368 of SEQ ID NO:1 in WO 2005/077042A2; (i) amino acids 439 to 447 of SEQ ID NO:1 in WO 2005/077042A2 (j) amino acids 462 to 475 of SEQ ID NO:1 in WO 2005/077042A2; (k) amino acids 478 to 486 of SEQ ID NO:1 in WO 2005/077042A2; and (1) amino acids 560 to 566 of SEQ ID NO:1 in WO 2005/077042A2.

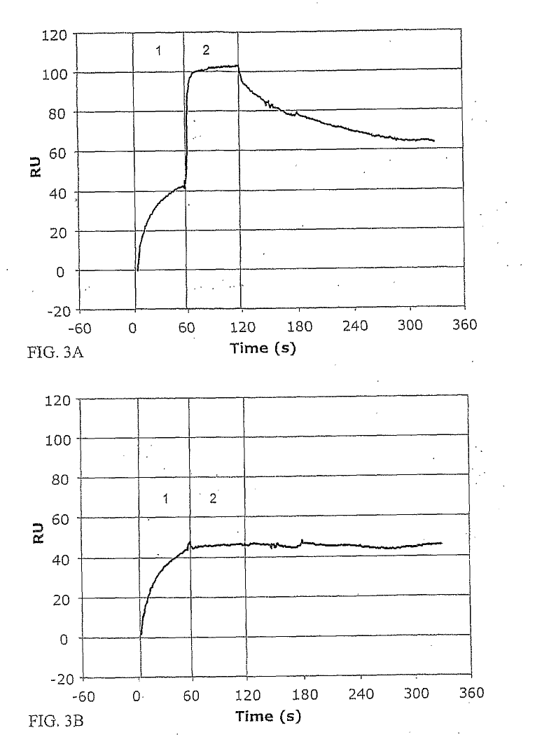

Further examples of suitable albumin, fragments and analogs for use in a ligand according to the invention are described in WO 03/076567A2, which is incorporated herein by reference in its entirety. In particular, the following albumin, fragments or variants can be used in the present invention:
Human serum albumin as described in WO 03/076567A2, e.g., in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al, FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, e.g., HA(1-373., HA(1-388), HA(1-389), HA(1-369), and HA(1-419) and fragments between 1-369 and 1-419;
An albumin fragment or variant as described in EP 399666, e.g., HA(1-177) and HA(1-200) and fragments between HA(1-X), where X is any number from 178 to 199.

Where a (one or more) half-life extending moiety (e.g., albumin, transferrin and fragments and analogues thereof) is used in the ligands of the invention, it can be conjugated using any suitable method, such as, by direct fusion to the IL-1R1-binding moiety (e.g., anti-IL-1R1 dAb or antibody fragment), for example by using a single nucleotide construct that encodes a fusion protein, wherein the fusion protein is encoded as a single polypeptide chain with the half-life extending moiety located N- or C-terminally to the IL-1R1 binding moiety. Alternatively, conjugation can be achieved by using a peptide linker between moieties, e.g., a peptide linker as described in WO 03/076567A2 or WO 2004/003019 (these linker disclosures being incorporated by reference in the present disclosure to provide examples for use in the present invention).

Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g., type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and β-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhances serum half-life in vivo also include proteins localized to the kidney (e.g., polycystin, type IV collagen, organic anion transporter K1, Heymaim's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1 GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

### Nucleic Acid Molecules, Vectors and Host Cells

The invention also provides isolated and/or recombinant nucleic acid molecules that encode the anti-IL-1R1 ligands and dAb monomers described herein, including dual specific ligands (e.g., ligands that bind IL-1R1 and serum albumin; ligands that bind IL-1R1 and TNFR1) and multispecific ligands (e.g., ligands that bind IL-1R1, serum albumin and TNFR1). The invention also provides isolated and/or recombinant nucleic acid molecules that encode a protease (e.g., (e.g., pepsin, trypsin, elastase, chymotrypsin, carboxypeptidase, cathepsin (e.g., cathepsin G) and proteinase 3) resistant dAb monomer or a ligand that comprises a protease resistant dAb monomer as described herein.

In certain embodiments, the isolated and/or recombinant nucleic acid comprises a nucleotide sequence that encodes a domain antibody (dAb) that specifically binds IL-1R, inhibits binding of IL-1 (e.g., IL-1α and/or IL-1β) and IL-1ra to IL-1R1, and comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence or a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), and DOM4-122-73 (SEQ ID NO:165).

In certain embodiments, the isolated and/or recombinant nucleic acid comprises a nucleotide sequence that encodes a domain antibody (dAb) monomer that specifically binds IL-1R1 and inhibits binding of IL-1 to the receptor, wherein said nucleotide sequence has at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), and DOM4-122-73 (SEQ ID NO:165).

In other embodiments, the isolated and/or recombinant nucleic acid comprises a nucleotide sequence that encodes a protease (e.g., (e.g., pepsin, trypsin, elastase, chymotrypsin, carboxypeptidase, cathepsin (e.g., cathepsin G) and proteinase 3) resistant dAb as described herein.

The invention also provides a vector comprising a recombinant nucleic acid molecule of the invention. In certain embodiments, the vector is an expression vector comprising one or more expression control elements or sequences that are operably linked to the recombinant nucleic acid of the invention The invention also provides a recombinant host cell comprising a recombinant nucleic acid molecule or vector of the invention. Suitable vectors (e.g., plasmids, phagmids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in procaryotic cells (e.g., lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol, 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In preferred embodiments, the host cell is a non-human host cell.

The invention also provides a method for producing a ligand (e.g., dAb monomer, dual-specific ligand, multispecific ligand) of the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

### Preparation of Immunoglobulin Based Ligands

Ligands (e.g., dual specific ligands, dAb monomers) according to the invention can be prepared according to previously established techniques, used in the field of antibody engineering, for the preparation of scFv, “phage” antibodies and other engineered antibody molecules. Techniques for the preparation of antibodies are for example described in the following reviews and the references cited therein: Winter & Milstein, (1991) Nature 349:293-299; Pluckthun (1992) Immunological Reviews 130:151-188; Wright et al., (1992) Crti. Rev. Immunol. 12:125-168; Holliger, P. & Winter, G. (1993) Curr. Op. Biotechn. 4, 446-449; Carter, et al. (1995) J. Hematother. 4, 463-470; Chester, K. A. & Hawkins, R. E. (1995) Trends Biotechn. 13, 294-300; Hoogenboom, H. R. (1997) Nature Biotechnol. 15, 125-126; Fearon, D. (1997) Nature Biotechnol. 15, 618-619; Plückthun, A. & Pack, P. (1997) Immunotechnology 3, 83-105; Carter, P. & Merchant, A. M. (1997) Curr. Opin. Biotechnol. 8, 449-454; Holliger, P. & Winter, G. (1997) Cancer Immunol. Immunother. 45, 128-130.

Suitable techniques employed for selection of antibody variable domains with a desired specificity employ libraries and selection procedures which are known in the art. Natural libraries (Marks et al. (1991) J. Mol. Biol., 222: 581; Vaughan et al. (1996) Nature Biotech., 14: 309) which use rearranged V genes harvested from human B cells are well known to those skilled in the art. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97) are prepared by cloning immunoglobulin V genes, usually using PCR. Errors in the PCR process can lead to a high degree of randomisation. V_{H} and/or V_{L} libraries may be selected against target antigens or epitopes separately, in which case single domain binding is directly selected for, or together.

### Library Vector Systems

A variety of selection systems are known in the art which are suitable for use in the present invention. Examples of such systems are described below.

Bacteriophage lambda expression systems may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. While such expression systems can be used to screen up to 10⁶ different members of a library, they are not really suited to screening of larger numbers (greater than 10⁶ members). Of particular use in the construction of libraries are selection display systems, which enable a nucleic acid to be linked to the polypeptide it expresses. As used herein, a selection display system is a system that permits the selection, by suitable display means, of the individual members of the library by binding the generic and/or target ligands.

Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encoding them) for the in vitro selection and amplification of specific antibody fragments that bind a target antigen (McCafferty et al., WO 92/01047). The nucleotide sequences encoding the variable regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pill or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature, 348: 552; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci. U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci. U.S.A., 85: 5879-5883; Chaudhary et al. (1990) Proc. Natl. Acad. Sci. U.S.A., 87: 1066-1070; McCafferty et al. (1990) supra; Clackson et al. (1991) Nature, 352: 624; Marks et al. (1991) J. Mol. Biol., 222: 581; Chiswell et al. (1992) Trends Biotech., 10: 80; Marks et al. (1992) J. Biol. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Other systems for generating libraries of polypeptides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection.

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### Library Construction

Libraries intended for selection, may be constructed using techniques known in the art, for example as set forth above, or may be purchased from commercial sources. Libraries which are useful in the present invention are described, for example, in WO99/20749. Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. Mutagenesis of nucleic acid sequences encoding structurally optimised polypeptides is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalysed by a thermostable, DNA-dependent DNA polymerase to amplify the target sequence of interest, is well known in the art. The construction of various antibody libraries has been discussed in Winter et al. (1994) Ann. Rev. Immunology 12, 433-55, and references cited therein.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler. The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenised, mismatch is required, at least in the first round of synthesis. The ability to optimise the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

### Combining Single Variable Domains

Immunoglobulin variable domains useful in the invention, once selected, may be combined by a variety of methods known in the art, including covalent and non-covalent methods. Preferred methods include the use of polypeptide linkers, as described, for example, in connection with scFv molecules (Bird et al., (1988) Science 242:423-426). Discussion of suitable linkers is provided in Bird et al. Science 242, 423-426; Hudson et al, Journal Immunol Methods 231 (1999) 177-189; Hudson et al, Proc Nat Acad Sci USA 85, 5879-5883. Linkers are preferably flexible, allowing the two single domains to interact. One linker example is a (Gly₄ Ser)ₙ linker, where n=1 to 8, e.g., 1, 2, 3, 4, 5, 6, 7 or 8. The linkers used in diabodies, which are less flexible, may also be employed (Holliger et al., (1993) Proc Nat Acad Sci (USA) 90:6444-6448). In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilise V_{H}-V_{H}, V_{L}-V_{L} or V_{H}-V_{L} dimers (Reiter et al., (1994) Protein Eng. 7:697-704) or by remodelling the interface between the variable domains to improve the “fit” and thus the stability of interaction (Ridgeway et al., (1996) Protein Eng. 7:617-621; Zhu et al., (1997) Protein Science 6:781-788). Other techniques for joining or stabilising variable domains of immunoglobulins, and in particular antibody V_{H} domains, may be employed as appropriate.

### Characterisation of Ligands

The binding of a ligand (e.g., dAb monomer, dual-specific ligand) to its specific antigen(s) or epitope(s) can be tested by methods which will be familiar to those skilled in the art and include ELISA. In a preferred embodiment of the invention binding is tested using monoclonal phage ELISA. Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

### Structure of Ligands

In the case that the immunoglobulin variable domains are selected from V-gene repertoires for instance using phage display technology as herein described, then these variable domains comprise a universal framework region, such that they may be recognised by a specific generic ligand as herein defined. The use of universal frameworks, generic ligands and the like is described in WO99/20749.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In general, nucleic acid molecules and vector constructs required for selection, preparation and formatting ligands may be constructed and manipulated as set forth in standard laboratory manuals, such as Sambrook et al. (1989) Molecular Cloning. A Laboratory Manual, Cold Spring Harbor, USA.

The manipulation of nucleic acids useful in the present invention is typically carried out in recombinant vectors. As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes and episomal vectors. Such vectors may be used for simple cloning and mutagenesis; alternatively gene expression vector is employed. A vector of use according to the invention may be selected to accommodate a polypeptide coding sequence of a desired size, typically from 0.25 kilobase (kb) to 40 kb or more in length. A suitable host cell is transformed with the vector after in vitro cloning manipulations. Each vector contains various functional components, which generally include a cloning (or “polylinker”) site, an origin of replication and at least one selectable marker gene. If a given vector is an expression vector, it additionally possesses one or more of the following: an enhancer element, promoter, transcription termination and signal sequences, each positioned in the vicinity of the cloning site, such that they are operatively linked to the gene encoding a ligand according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g., SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g., ampicillin, neomycin, methotrexate or tetracycline, complement auxotrophic deficiencies, or supply critical nutrients not available in the growth media.

Since the replication of vectors encoding a ligand according to the present invention is most conveniently performed in E. Coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Delgarno sequence operably linked to the coding sequence.

The preferred vectors are expression vectors that enable the expression of a nucleotide sequence corresponding to a polypeptide library member. Thus, selection with the first and/or second antigen or epitope can be performed by separate propagation and expression of a single clone expressing the polypeptide library member or by use of any selection display system. As described above, the preferred selection display system is bacteriophage display. Thus, phage or phagemid vectors may be used, e.g. pIT1 or pIT2. Leader sequences useful in the invention include pelB, stlI, ompA, phoA, bla and pelA. One example are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of an expression cassette that consists (N to C terminal) of a pelB leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors encoding ligands according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

### Skeletons

Skeletons may be based on immunoglobulin molecules or may be non-immunoglobulin in origin as set forth above. Preferred immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

### Protein Scaffolds

Each epitope binding domain comprises a protein scaffold and one or more CDRs which are involved in the specific interaction of the domain with one or more epitopes. Advantageously, an epitope binding domain according to the present invention comprises three CDRs. Suitable protein scaffolds include any of those selected from the group consisting of the following: those based on immunoglobulin domains, those based on fibronectin, those based on affibodies, those based on CTLA4, those based on chaperones such as GroEL, those based on lipocallin and those based on the bacterial Fc receptors SpA and SpD. Those skilled in the art will appreciate that this list is not intended to be exhaustive.

### Scaffolds for Use in Constructing Ligands

### Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

Libraries of ligands and/or domains can be designed in which certain loop lengths and key residues have been chosen to ensure that the main-chain conformation of the members is known. Advantageously, these are real conformations of immunoglobulin superfamily molecules found in nature, to minimise the chance that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on ligand sequences and to choose residues for diversification which do not affect the canonical structure. It is known that, in the human Vκ domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that V_{κ} and V_{λ} domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the dual-specific ligands of the invention possess a single known main-chain conformation.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing ligands (e.g., dAbs) or libraries thereof the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e., between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS 1 (79% of the expressed repertoire), H2-CS 3 (46%), L1-CS 2 of V_{κ} (39%), L2-CS 1 (100%), L3-CS 1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

### Diversification of the Canonical Sequence

Having selected several known main-chain conformations or, preferably a single known main-chain conformation, ligands (e.g., dAbs) or libraries for use in the invention can be constructed by varying the binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or are preferably selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomisation has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

Preferably, only the residues which are directly involved in creating or modifying the desired function of the molecule are diversified. For many molecules, the function will be to bind a target and therefore diversity should be concentrated in the target binding site, while avoiding changing residues which are crucial to the overall packing of the molecule or to maintaining the chosen main-chain conformation.

### Diversification of the Canonical Sequence as it Applies to Antibody Domains

In the case of antibody based ligands (e.g., dAbs), the binding site for the target is most often the antigen binding site. Thus, preferably only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library for use according to the invention. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire can be created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

Naive repertoires of binding domains for the construction of ligands in which some or all of the residues in the antigen binding site are varied are known in the art. (See, WO 2004/058821, WO 2004/003019, and WO 03/002609). The “primary” library mimics the natural primary repertoire, with diversity restricted to residues at the centre of the antigen binding site that are diverse in the germline V gene segments (germline diversity) or diversified during the recombination process (junctional diversity). Those residues which are diversified include, but are not limited to, H50, H52, H52a, H53, H55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated). Those residues which are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use in the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favours certain amino acid residues. If the amino acid composition of the ten most diverse positions in each of the V_{H}, V_{κ} and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens or epitopes and may help to explain the required promiscuity of antibodies in the primary repertoire.

Since it is preferable to mimic this distribution of amino acids, the distribution of amino acids at the positions to be varied preferably mimics that seen in the antigen binding site of antibodies. Such bias in the substitution of amino acids that permits selection of certain polypeptides (not just antibody polypeptides) against a range of target antigens is easily applied to any polypeptide repertoire. There are various methods for biasing the amino acid distribution at the position to be varied (including the use of tri-nucleotide mutagenesis, see WO97/08320), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

### Therapeutic and Diagnostic Compositions and Uses

The invention provides compositions comprising a ligand of the invention (e.g., dual-specific ligand, multi-specific ligand, dAb monomer) and a pharmaceutically acceptable carrier, diluent or excipient, and therapeutic and diagnostic methods that employ the ligands or compositions of the invention. Ligands (e.g., dual-specific ligands, multispecific ligands, dAb monomers) according to the method of the present invention may be employed in in vivo therapeutic and prophylactic applications, in vivo diagnostic applications and the like.

Therapeutic and prophylactic uses of ligands (e.g., multispecific ligands, dual-specific ligands, dAb monomers) of the invention involve the administration of ligands according to the invention to a recipient mammal, such as a human. Dual-specific and multi-specific ligands (e.g., dual-specific antibody formats) bind to multimeric antigen with great avidity. Dual- or multi-specific ligands can allow the cross-linking of two antigens, for example in recruiting cytotoxic T-cells to mediate the killing of tumour cell lines.

Substantially pure ligands, for example dAb monomers, of at least 90 to 95% homogeneity are preferred for administration to a mammal, and 98 to 99% or more homogeneity is most preferred for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

For example, the ligands (e.g., dAb monomers), of the present invention will typically find use in preventing, suppressing or treating inflammation or inflammatory states including acute inflammatory diseases and/or chronic inflammatory diseases. The ligands (e.g., dAb monomers), of the present invention can also be administered to inhibit biological processes that are induced by binding of IL-1 (e.g., IL-1α and/or IL-1β) to IL-1R1.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

The ligands of the invention, including dAb monomers, can be administered to prevent, suppress or treat a chronic inflammatory disease, allergic hypersensitivity, cancer, bacterial or viral infection, autoimmune disorders (which include, but are not limited to, Type I diabetes, asthma, multiple sclerosis, systemic lupus erythematosus, inflammatory bowel disease (e.g., Crohn's disease, ulcerative colitis), myasthenia gravis and Behcet's syndrome), psoriasis, endometriosis, and abdominal adhesions (e.g., post abdominal surgery).

The ligands of the invention, including dAb monomers, can be administered to prevent, suppress or treat lung inflammation, chronic obstructive respiratory disease (e.g., chronic bronchitis, chronic obstructive bronchitis, emphysema), asthma (e.g., steroid resistant asthma), pneumonia (e.g., bacterial pneumonia, such as Staphylococcal pneumonia), hypersensitivity pneumonitis, pulmonary infiltrate with eosinophilia, environmental lung disease, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, chronic bronchitis, emphysema, eosinophilic pneumonia, idiopathic pulmonary fibrosis, invasive pneumococcal disease (IPD), influenza, nontuberculous mycobacteria, pleural effusion, pneumoconiosis, pneumocytosis, pulmonary actinomycosis, pulmonary alveolar proteinosis, pulmonary anthrax, pulmonary edema, pulmonary embolus, pulmonary inflammation, pulmonary histiocytosis X (eosinophilic granuloma), pulmonary hypertension, pulmonary nocardiosis, pulmonary tuberculosis, pulmonary veno-occlusive disease, rheumatoid lung disease, sarcoidosis, Wegener's granulomatosis, and non-small cell lung carcinoma.

The ligands of the invention, including dAb monomers, can be administered to prevent, suppress or treat influenza, RSV-associated respiratory disease and viral lung (respiratory) disease.

The ligands of the invention, including dAb monomers, can be administered to prevent, suppress or treat osteoarthritis or inflammatory arthritis. “Inflammatory arthritis” refers to those diseases of joints where the immune system is causing or exacerbating inflammation in the joint, and includes rheumatoid arthritis, juvenile rheumatoid arthritis, and spondyloarthropathies, such as ankylosing spondylitis, reactive arthritis, Reiter's syndrome, psoriatic arthritis, psoriatic spondylitis, enteropathic arthritis, enteropathic spondylitis, juvenile-onset spondyloarthropathy and undifferentiated spondyloarthropathy. Inflammatory arthritis is generally characterized by infiltration of the synovial tissue and/or synovial fluid by leukocytes.

Ligands according to the invention (e.g., dual-specific ligands, multispecific ligands, dAb monomers) which bind to extracellular targets involved in endocytosis (e.g., Clathrin) can be endocytosed, enabling access to intracellular targets. In addition, dual or multispecific ligands, provide a means by which a binding domain (e.g., a dAb monomer) that is able to bind to an intracellular target can be delivered to an intracellular environment. This strategy requires, for example, a dual-specific ligand with physical properties that enable it to remain functional inside the cell. Alternatively, if the final destination intracellular compartment is oxidising, a well folding ligand may not need to be disulphide free.

Advantageously, dual- or multi-specific ligands may be used to target cytokine receptors and other molecules which cooperate synergistically in therapeutic situations in the body of an organism. The invention therefore provides a method for synergising the activity of two or more binding domains (e.g., dAbs) that bind cytokine receptors or other molecules, comprising administering a dual- or multi-specific ligand capable of binding to said two or more molecules (e.g., cytokine receptors). In this aspect of the invention, the dual- or multi-specific ligand may be any dual- or multi-specific ligand, for example, this aspect of the invention relates to combinations of V_{H} domains and V_{L} domains, V_{H} domains only and V_{L} domains only.

Synergy in a therapeutic context may be achieved in a number of ways. For example, target combinations may be therapeutically active only if both targets are targeted by the ligand, whereas targeting one target alone is not therapeutically effective. In another embodiment, one target alone may provide some therapeutic effect, but together with a second target the combination provides a synergistic increase in therapeutic effect (a more than additive effect).

Animal model systems which can be used to screen the effectiveness of the ligands of the invention in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179). Other suitable models are described herein.

Generally, the ligands will be utilised in purified form together with pharmacologically appropriate carriers. Typically, these carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present. Formulation will depend on the route of administration, a variety of suitable formulations can be used, including extended release formulations. (See, e.g., Mack (1982) Remington's Pharmaceutical Sciences, 16th Edition.)

The ligands (e.g., dAb monomers) can be administered and or formulated together with one or more additional therapeutic or active agents. When a ligand is administered with an additional therapeutic agent, the ligand can be administered before, simultaneously with or subsequent to administration of the additional agent. Generally, the ligand (e.g., dAb monomer) and additional agent are administered in a manner that provides an overlap of therapeutic effect. Additional agents that can be administered or formulated with the ligand of the invention include, for example, various immunotherapeutic drugs, such as cylcosporine, methotrexate, adriamycin or cisplatinum, antibiotics, antimycotics, anti-viral agents and immunotoxins. For example, when the antagonist is administered to prevent, suppress or treat lung inflammation or a respiratory disease, it can be administered in conjunction with phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short acting anticholinergics (e.g., ipratropium bromide and oxitropium bromide), long-acting anticholinergics (e.g., tiotropium), theophylline (e.g., short acting formulation, long acting formulation), inhaled steroids (e.g., beclomethasone, beclometasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol.

When the antagonist is administered to prevent, suppress or treat arthritis (e.g., inflammatory arthritis (e.g., rheumatoid arthritis)), it can be administered in conjunction with a disease modifying anti-rheumatic agent (e.g., methotrexate, hydroxychloroquine, sulfasalazine, leflunomide, azathioprine, D-penicillamine, gold (oral or intramuscular), minocycline, cyclosporine, staphylococcal protein A), nonsteroidal anti-inflammatory agent (e.g., COX-2 selective NSAIDS such as rofecoxib), salicylates, glucocoricoids (e.g., predisone) and analgesics.

Pharmaceutical compositions can include “cocktails” of various cytotoxic or other agents in conjunction with ligands of the present invention, or even combinations of ligands according to the present invention having different specificities, such as ligands selected using different target antigens or epitopes, whether or not they are pooled prior to administration.

The route of administration of pharmaceutical compositions according to the invention may be any of those commonly known to those of ordinary skill in the art. For therapy, including without limitation immunotherapy, the selected ligands thereof of the invention can be administered to any patient in accordance with standard techniques. The administration can be by any appropriate mode, including parenterally (e.g., intravenous, intramuscular, intraperitoneal, intra-articular, intrathecal), transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. Administration can be local (e.g., local delivery to the lung by pulmonary administration, e.g., intranasal administration) or systemic as indicated.

The ligands of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g., with conventional immunoglobulins, IgM antibodies tend to have greater activity loss than IgG antibodies) and that use levels may have to be adjusted upward to compensate.

The compositions containing the present antagonists (e.g., ligands) or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose.” For example, for treating lung inflammation and/or a respiratory disease, a sputum-inhibiting amount, a bronchial biopsy inflammation-inhibiting amount, a dyspnoea-inhibiting amount, a forced expiratory volume in one second (FEV (1)) increasing amount, an improvement in health status increasing amount, as quantified in a suitable questionnaire such as the St. George's Respiratory Questionnaire (e.g., an improvement score of 4 points) can be administered. In another example, for treating arthritis (e.g., inflammatory arthritis (e.g., rheumatoid arthritis)), an amount sufficient to achieve a 20% or greater improvement in at least 3 of the American College of Rheumatology core set measures can be administered (Felson et al., Arthritis and Rheumatism, 38:727-735 (1995)).

Amounts needed to achieve this dosage will depend upon the severity of the disease and the general state of the patient, including the patients age, sex, weight, general health (e.g., the state of the patients immune system). Based on these and other appropriate criteria, the skilled clinician can determine the appropriate amount of ligand to be administered. Generally the amount can range from 0.005 to 5.0 mg of ligand per kilogram of body weight, with doses of 0.05 to 2.0 mg/kg/dose being more commonly used. For prophylactic applications, compositions containing the present ligands or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. The ligand of the invention can be administered up to four times per day, twice weekly, once weekly, once every two weeks, once a month, or once every two months, at a dose off, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the ligand is administered to treat, suppress or prevent a chronic inflammatory disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status—score ranges from 32 to 224, with higher scores indicating a better quality of life), the Quality of Life Rheumatoid Arthritis Scale, the American College of Rheumatology core set measures, or other accepted clinical assessment scale as known in the field. A sustained (e.g., one day or more, preferably longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing an ligand or cocktail thereof according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g., antibodies, cell-surface receptors or binding proteins thereof whereby the undesired cells are killed or otherwise removed from the blood for return to the mammal in accordance with standard techniques.

A composition containing an antagonist (e.g., ligand) according to the present invention may be utilised in prophylactic and therapeutic settings to aid in the alteration, inactivation, killing or removal of a select target cell population in a mammal.

In one embodiment, the invention is a method for treating, suppressing or preventing a chronic inflammatory disease, comprising administering to a mammal in need thereof a therapeutically-effective dose or amount of a ligand of the invention.

In one embodiment, the invention is a method for treating, suppressing or preventing arthritis (e.g., Inflammatory arthritis (e.g., rheumatoid arthritis, juvenile rheumatoid arthritis, and spondyloarthropathies, such as ankylosing spondylitis, reactive arthritis, Reiter's syndrome, psoriatic arthritis, psoriatic spondylitis, enteropathic arthritis, enteropathic spondylitis, juvenile-onset spondyloarthropathy and undifferentiated spondyloarthropathy)) comprising administering to a mammal in need thereof a therapeutically-effective dose or amount of a ligand of the invention.

In another embodiment, the invention is a method for treating, suppressing or preventing inflammatory bowel disease (e.g., Crohn's disease, ulcerative colitis) comprising administering to a mammal in need thereof a therapeutically-effective dose or amount of a ligand of the invention.

### EXAMPLES

### Example 1

### Methods

### Selections and Screening

For primary selections, 4G-K2 library of Vκ dAbs was panned against IL-1R1-Fc fusion protein (Axxora, Nottingham, UK). Domain antibodies from the primary selection were subjected to three further rounds of selection. Round 1 was performed using protein G coated magnetic beads (Dynal, Norway) and 100 nM IL-1R1-Fc; round 2 was performed using anti-human IgG beads (Novagen, Merck Biosciences, Nottingham, UK) and 10 nM IL-1R1-Fc; and round 3 was performed using protein G beads and 1 nM IL-1R1-Fc. (Henderikx et al., Selection of antibodies against biotinylated antigens. Antibody Phage Display: Methods and protocols, Ed. O'Brien and Atkin, Humana Press (2002).) Elution at each stage was with 1 mg/ml trypsin-phosphate buffered saline (PBS). For affinity maturation selections, the above method was used, but with the following modifications: two rounds of selection were performed using protein G beads, round 1 using 1 nM IL-1R1-Fc, and round 2 using 100 pM IL-1R1-Fc. Phage vectors from selection outputs (rounds 2 and 3) were isolated by plasmid preps (Qiagen) and dAb inserts were released by restriction digest with Sal I and Not I. This inserts were ligated into a phage expression vector (Sal I/Not I cut pDOM5) and used to transform E. coli strain HB2151 for soluble expression and screening of dAbs.

### Supernatant Receptor Binding Assay (RBA)

Single transformed E. coli colonies were picked into 96-well plates containing 2×TY supplemented with 100 μg/ml carbenicillin and 0.1% (w/v) glucose, grown at 37° C. to ˜OD₆₀₀=0.9 and induced with 1 mM IPTG. Supernatants from overnight inductions at 30° C. were screened in a receptor binding assay for the ability to inhibit binding of IL-1β to IL-1R1 captured on an ELISA plate. Briefly, MaxiSorp™ immunoassay plates (Nunc, Denmark) were incubated overnight with anti-IL-1R1 mouse monoclonal antibody (R&D Systems, Minneapolis, USA). The wells were washed with phosphate buffered saline (PBS) containing 0.1% (v/v) Tween-20 and then blocked with 1% (w/v) BSA in PBS before being incubated with recombinant IL-1R1 (500 ng/ml, R&D Systems). The E. coli culture supernatants containing dAbs to be screened were placed in the washed wells of the assay plate, the plate was incubated for 30 min at room temperature, then IL-1β (4 ng/ml, R&D Systems) was added to each well and mixed. IL-1β binding was detected using biotinylated anti-IL-1β antibody (R&D Systems), followed by peroxidase labelled anti-biotin antibody (Stratech, Soham, UK) and then, incubation with 3,3′,5,5′-tetramethylbenzidine (TMB) substrate (ILL, Gaithersburg, USA). The reaction was stopped by the addition of HCl and the absorbance was read at 450 nm. Anti-IL-1R1 dAb activity caused a decrease in IL-1β binding and therefore a decrease in absorbance compared with the IL-1β only control.

### Cell Assay

Isolated dAbs were tested for their ability to inhibit IL-1-induced IL-8 release from cultured MRC-5 cells (ATCC catalogue no. CCL-171). Briefly, 5000 trypsinised MRC-5 cells in RPMI media were placed in the well of a tissue-culture microtitre plate and mixed with IL-1α or β (R&D Systems, 200 pg/ml final concentration) and a dilution of the dAb to be tested. The mixture was incubated overnight at 37° C. and IL-8 released by the cells into to culture media was quantified in an ELISA (DuoSet®, R&D Systems). Anti-IL-1R1 dAb activity caused a decrease in IL-1 binding and a corresponding reduction in IL-8 release.

### Human Whole Blood Assay

Whole human blood was incubated with a dilution series of the dAb to be tested, and the mixture was incubated for 30 min at 37° C./5% CO₂. Next, 270 or 900 pM (final concentration) IL-1α or IL-1β was added and the mixture, and then the mixtures was incubated at 37° C./5% CO₂ for an additional 20 hours. The blood was then centrifuged (500×g, 5 min) and the IL-6 released into the supernatant was quantified in an ELISA (DuoSet®, R&D Systems). Anti-IL-1R1 dAb activity caused a decrease in IL-1 binding and a corresponding reduction in IL-6 release.

### Off-Rate Screening

These experiments were performed on a BIACORE 3000 surface plasmon resonance instrument, using a CM5 chip (Biacore) coupled with ˜600 RU of IL-1R1 (R&D Systems). Analytes were passed over the IL-1R1-coated flow-cell, with in-line referencing against a blank flow-cell, at a flow rate of 30 μl/min in HBS-EP running buffer (Biacore). Ten microlitres of supernatant containing soluble dAb was diluted 1:1 in running buffer, injected (Kinject) at 10 μl/min flow rate and allowed to dissociate in buffer. Clones with improved off-rates compared to parental clones were identified by eye, or by measurement using BIAevaluation software v4.1.

### IL-1ra Competition by Surface Plasmon Resonance

These experiments were performed on a BIACORE 3000 surface plasmon resonance instrument, using a CM5 chip (Biacore) coupled with ˜600 RU of IL-1R1 (R&D Systems). Analytes were passed over the antigen-coated flow-cell, with in-line referencing against a blank flow-cell, at a flow rate of 30 μl/min in HBS-EP running buffer (Biacore). IL-1ra (100 nM, R&D Systems) was injected for 60 seconds, followed immediately by a 60 second injection of 200 nM DOM4-130-3 dAb or 100 nM IL-1α, using the co-inject facility.

### IL-1ra Competition ELISA

A MaxiSorp™ immunoassay plate (Nunc, Denmark) was coated overnight with 1 μg/ml IL-1R1-Fc, then washed three times with PBS before blocking with 1% (v/v) Tween 20 in PBS. The plates were washed again, before the addition of 500 pM IL-1ra mixed with a dilution series of DOM4-130-3 or IL-1α. Binding of IL-1ra to the receptor was detected using biotinylated anti-IL-1ra antibody (DuoSet®, R&D Systems), followed by streptavidin-HRP and developed with 3,3′,5,5′-tetramethylbenzidine (TMB) substrate (KPL, Gaithersburg, USA) as described above. Competition with IL-1ra for binding to IL-1R1 was indicated by a reduction in A₄₅₀ compared to control wells containing no IL-1ra.

### Affinity Maturation Phage Library Construction

Two types of libraries were constructed: CDR-re-diversified libraries and error-prone libraries. For the former type of library, PCR reactions were performed, using degenerate oligonucleotides containing NNK or NNS codons, to diversify the required positions in the dAb to be affinity matured. Assembly PCR was then used to generate a full length diversified insert. For the error-prone library, plasmid DNA encoding the dAb to be affinity matured was amplified by PCR, using the GeneMorph® II Random Mutagenesis kit (Stratagene). Inserts produced by either method were digested with Sal I and Not I and used in a ligation reaction with cut phage vector. This ligation was then used to transform E. coli strain TB1 by electroporation and the transformed cells were plated on 2×TY agar containing 15 μg/ml tetracycline, yielding library sizes of >1×10⁸ clones.

### Results

### Primary Selection and Screening

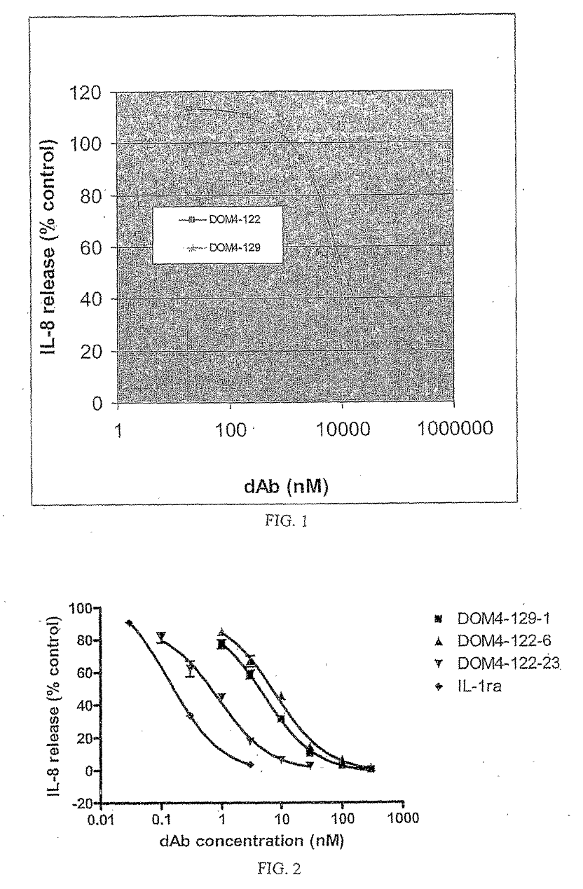

Primary phage selections were performed using the 4G-K2 library and outputs sub-cloned into a soluble expression vector (pDOM5). dAb clones that inhibited binding of IL-1 to IL-1R1 were identified by supernatant RBA, then expressed, purified by protein L and tested for their ability to inhibit IL-1-induced IL-8 release in an MRC-5 cell assay. FIG. 1 shows a dose-response curve for anti-IL-1R1 dAbs DOM4-122 and DOM4-129 in such a cell assay. The neutralizing dose 50 (ND₅₀) for each dAb was about 1 μM in the assay. DOM4-122 and DOM4-129 have the same amino acid sequence in CDRs 1 and 2, and have two out of five amino acid residues identical in CDR3, and therefore were predicted to bind to the same epitope on the receptor.

### Affinity Maturation

### Stage I Maturation

Using DOM4-122 as a template, a maturation library was constructed with diversity encoding all 20 amino acids at positions 28, 30, 31, 92 and 93. In parallel, DOM4-129 was affinity matured by error-prone PCR mutagenesis. The resulting phage libraries were used in soluble selections against IL-1R1-Fc. Round 2 and 3 selection outputs were cloned into phage expression vector (pDOM5), dAbs were expressed in E. coli, and dAbs in the expression supernatants were screened for improved off-rates compared to parental dAb. Clones with improved off-rates were expressed, purified and tested in the MRC-5/IL-8 assay. FIG. 2 depicts a dose-response curve for improved variants DOM4-122-6 and DOM4-129-1, which both had an ND₅₀ values of ˜10 nM.

### Stage II Maturation

Stage I affinity maturation of anti-IL-1R1 dAb DOM4-122 (ND₅₀˜1 μM) by re-diversification of CDRs 1 and 3 yielded DOM4-122-6 (˜10 nM). Maturation of DOM4-129 (˜1 μM) by error-prone PCR produced DOM4-129-1 (˜10 nM). DOM4-129-1 and DOM4-122-6 gained a mutation, L46F, in common during maturation while DOM4-129-1 has an additional mutation, S56R. Both changes were frequently found in clones isolated from maturation selections, therefore the S56R mutation was introduced into DOM4-122-6, yielding DOM4-122-23. This combination mutant dAb has an ND₅₀ of approximately 1 nM (FIG. 2). An additional point mutation, K45M, gained in both DOM4-122 and DOM4-129 was shown to be non-essential when reverted to germline in DOM4-122-23, yielding DOM4-122-24.

Epitopic Specificity of dAbs

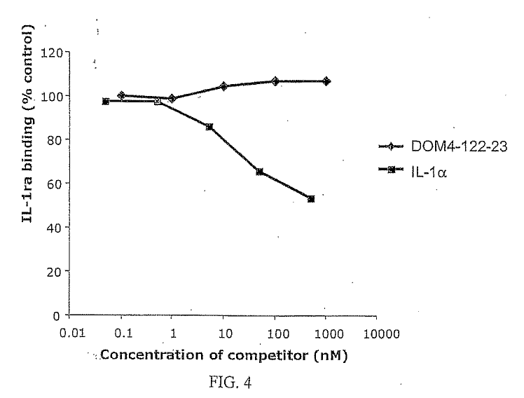

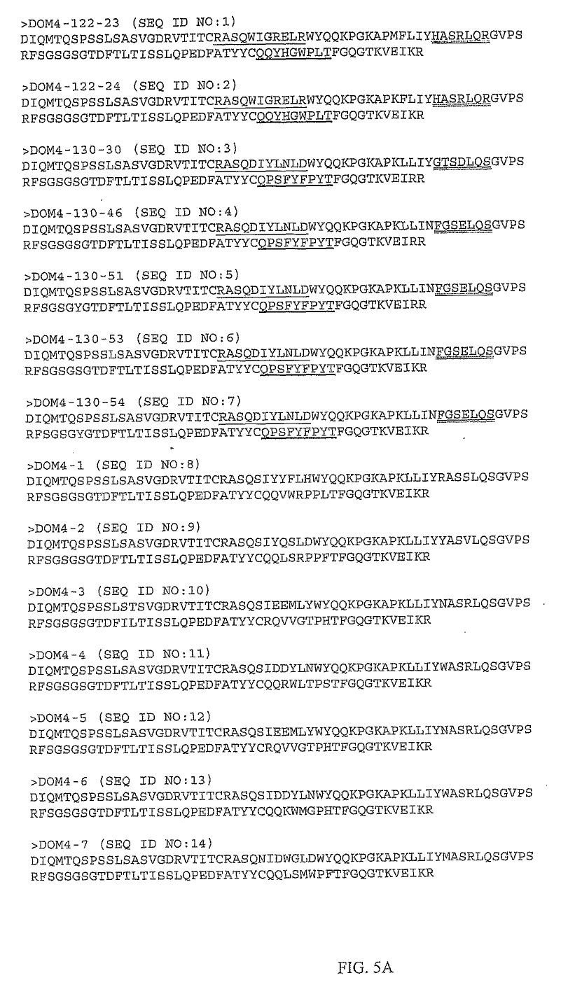

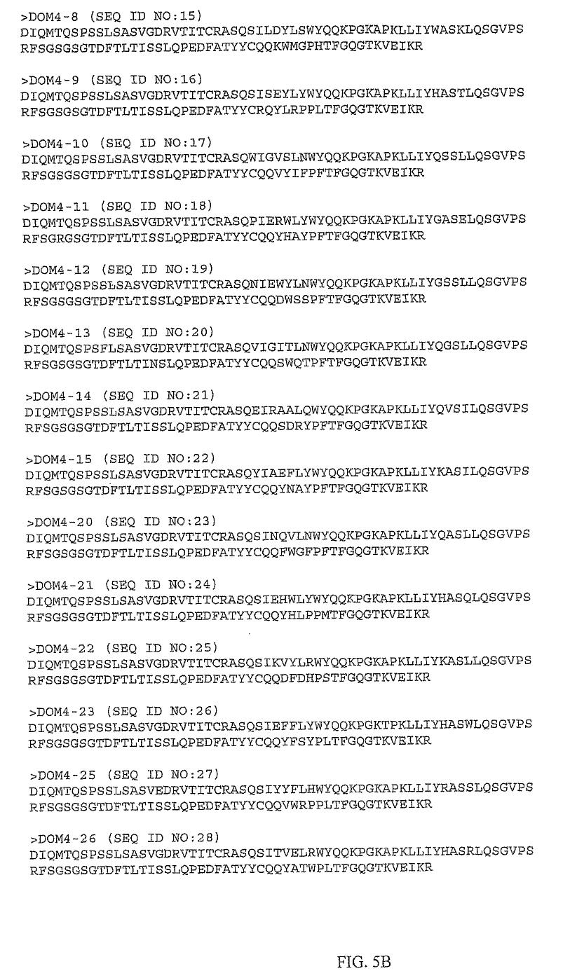

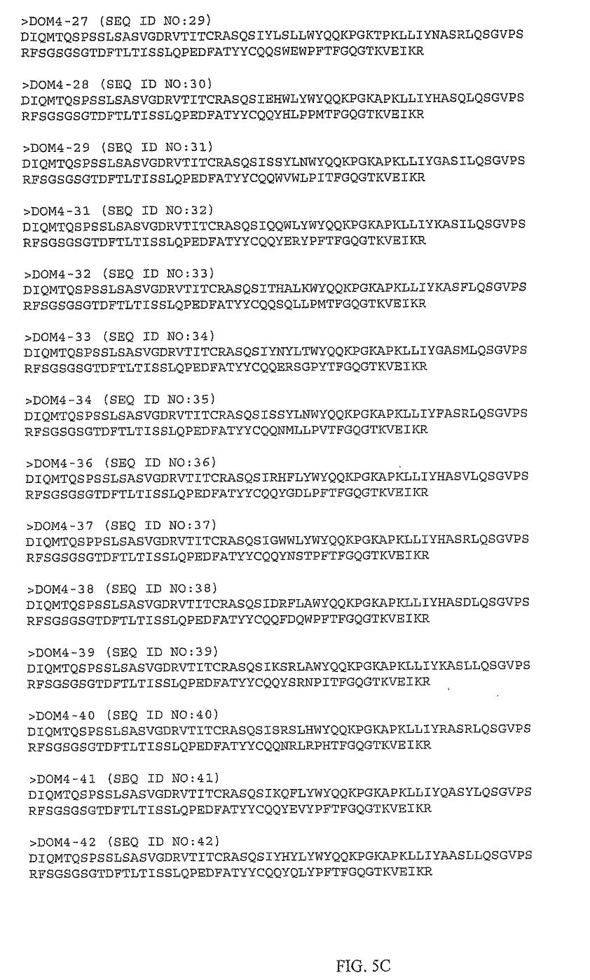

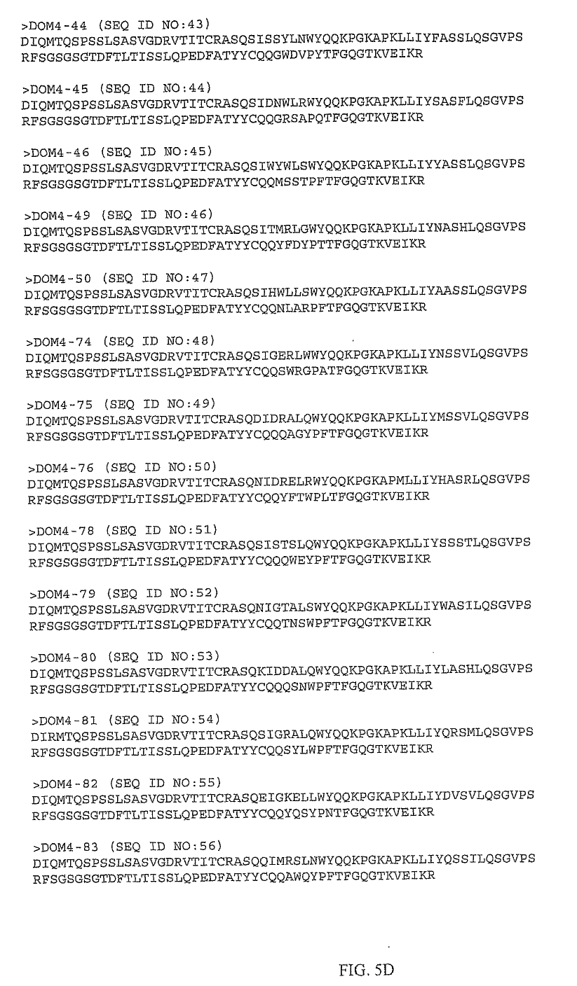

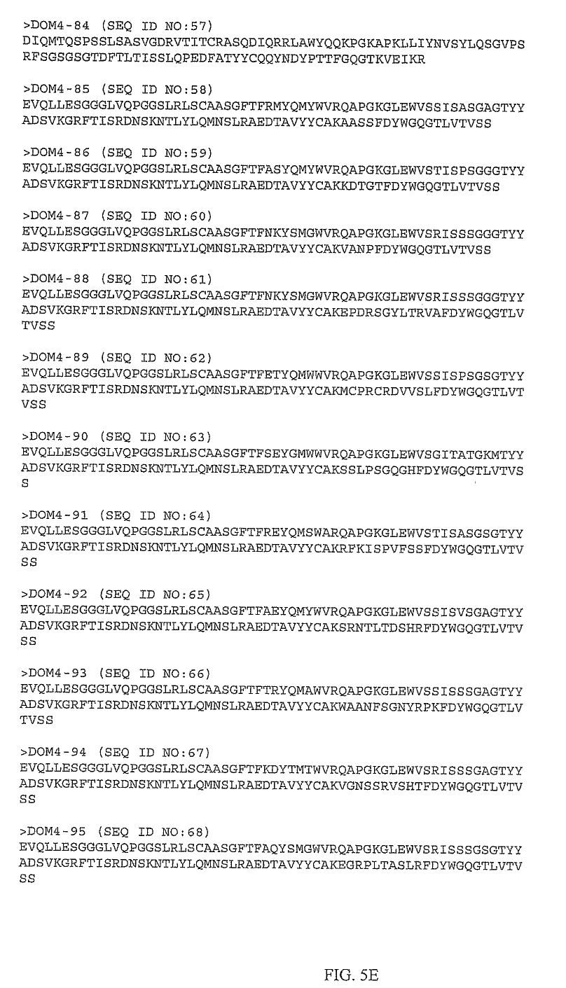

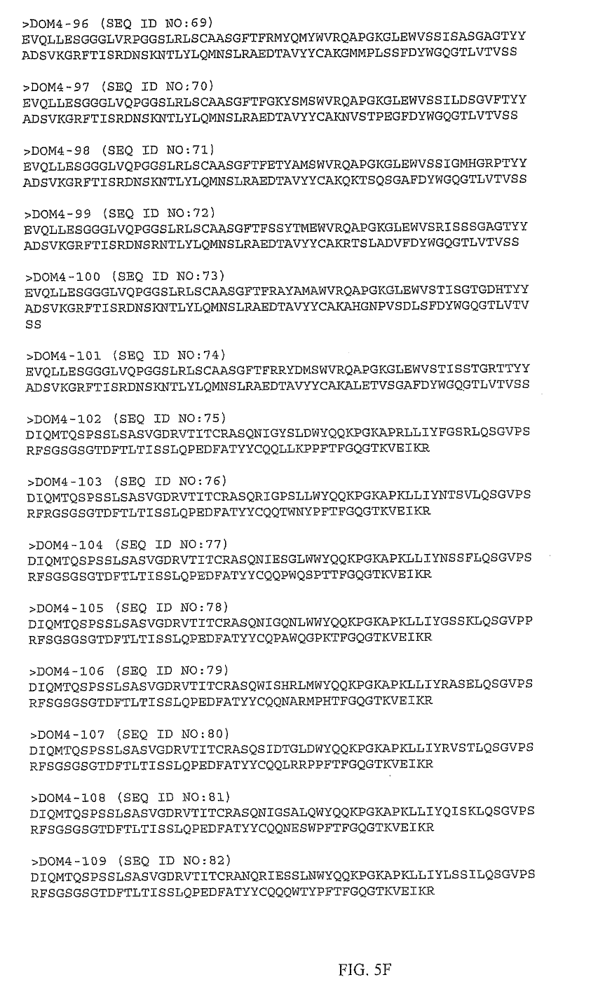

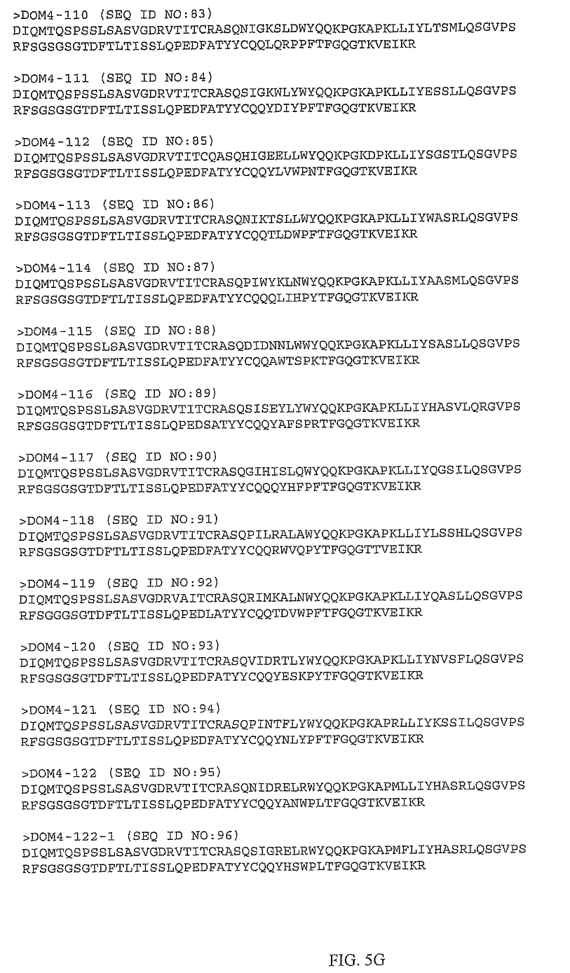

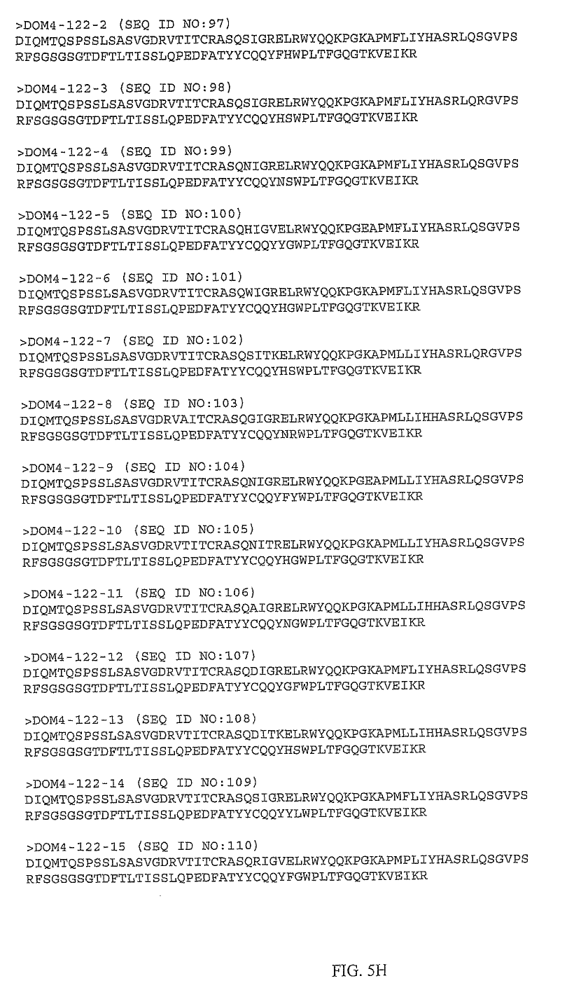

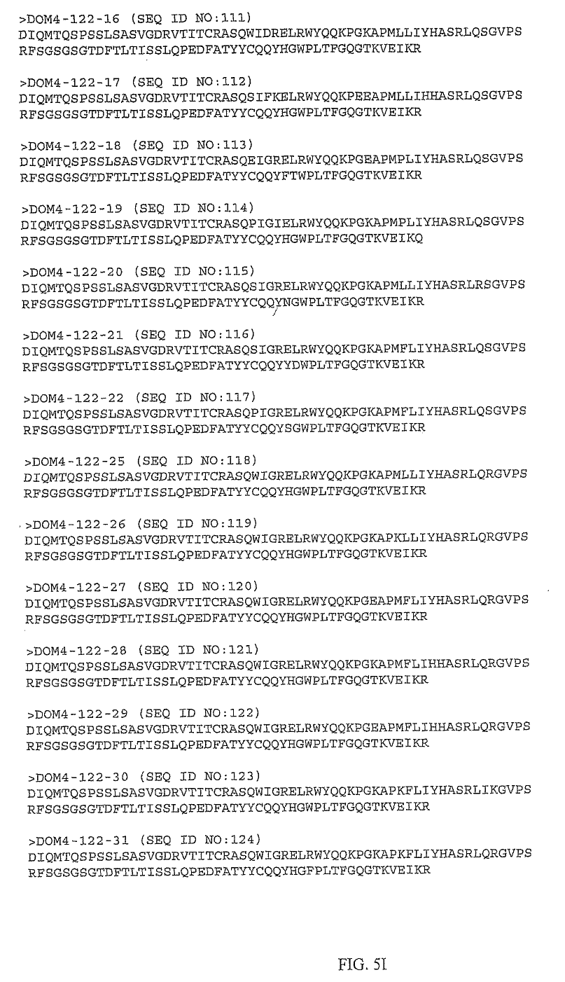

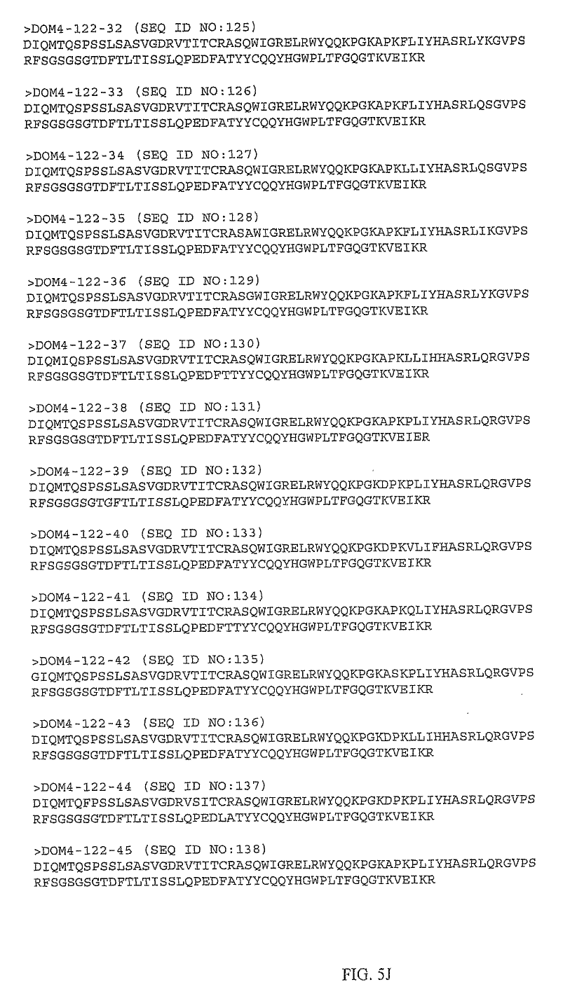

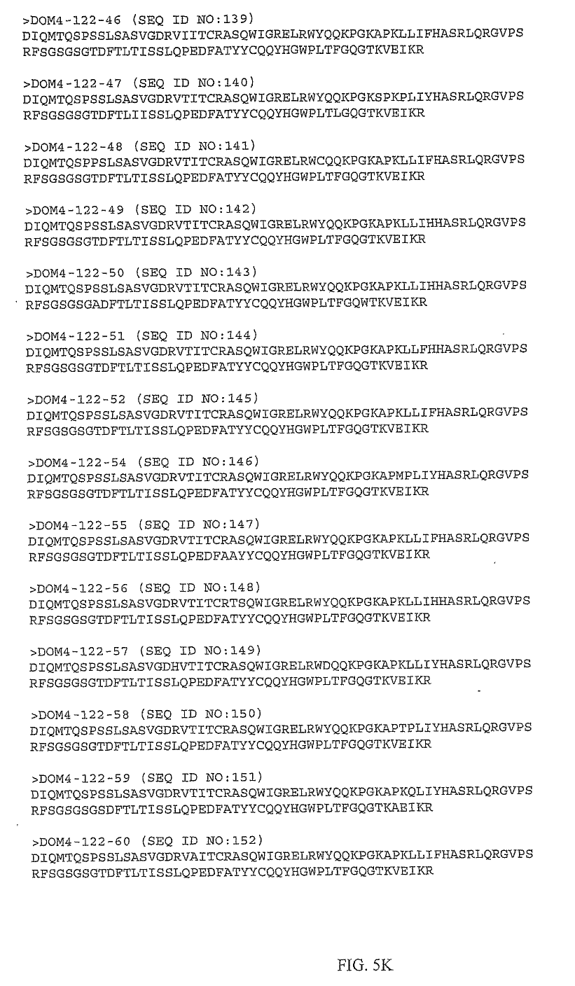

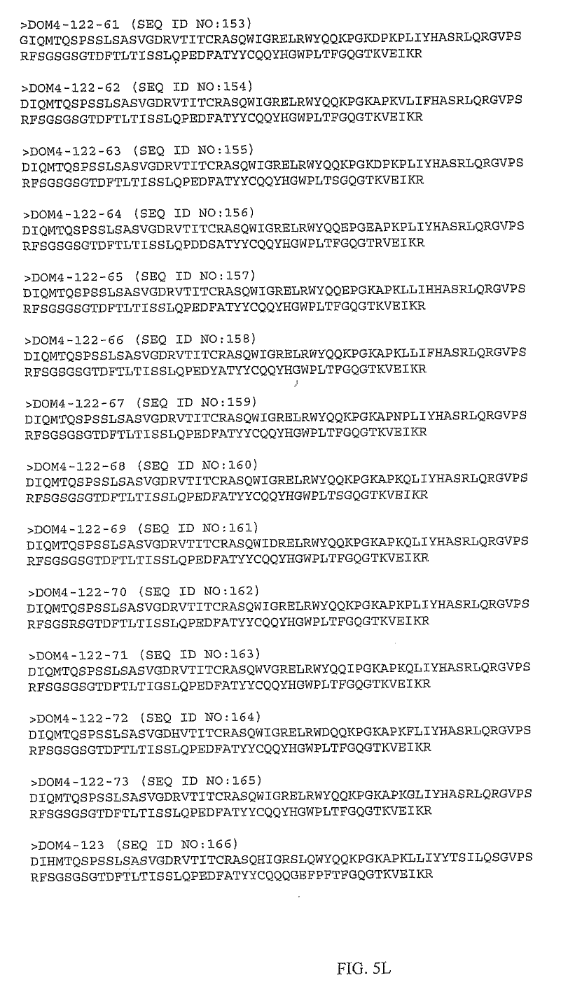

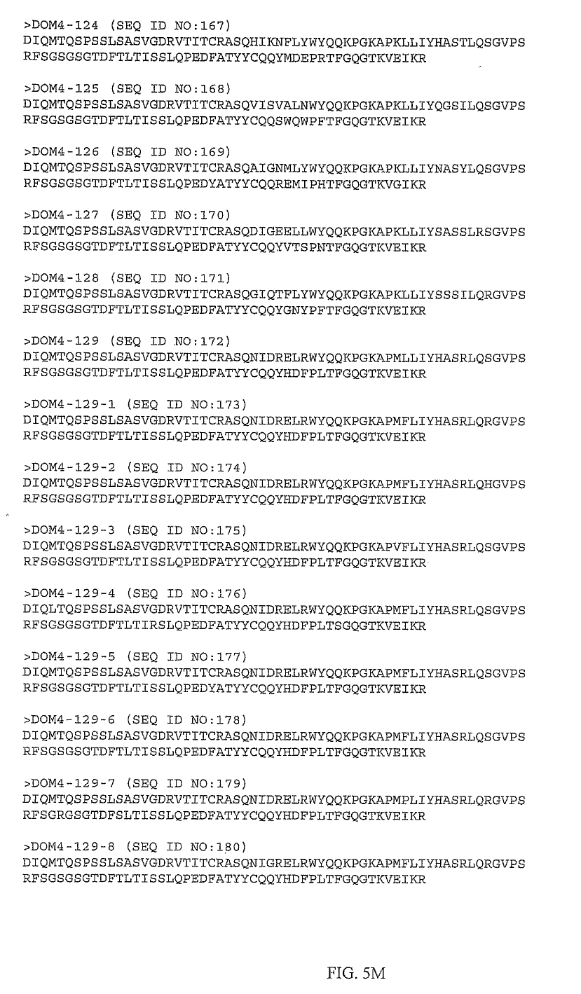

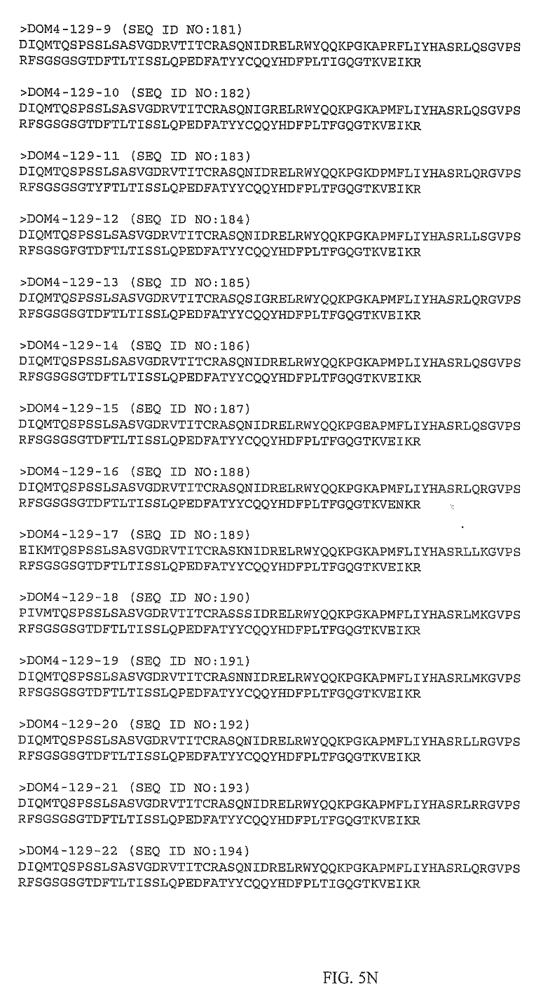

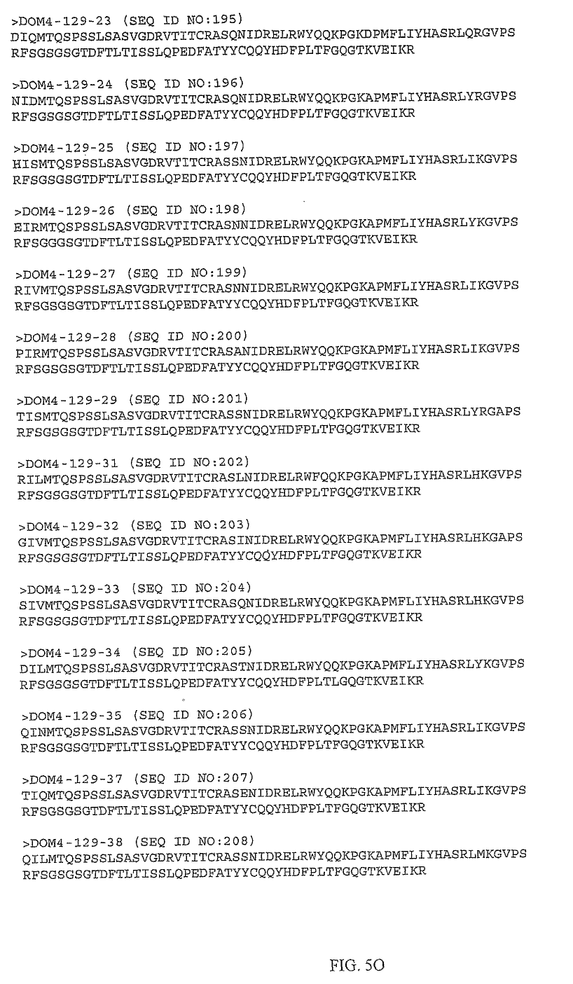

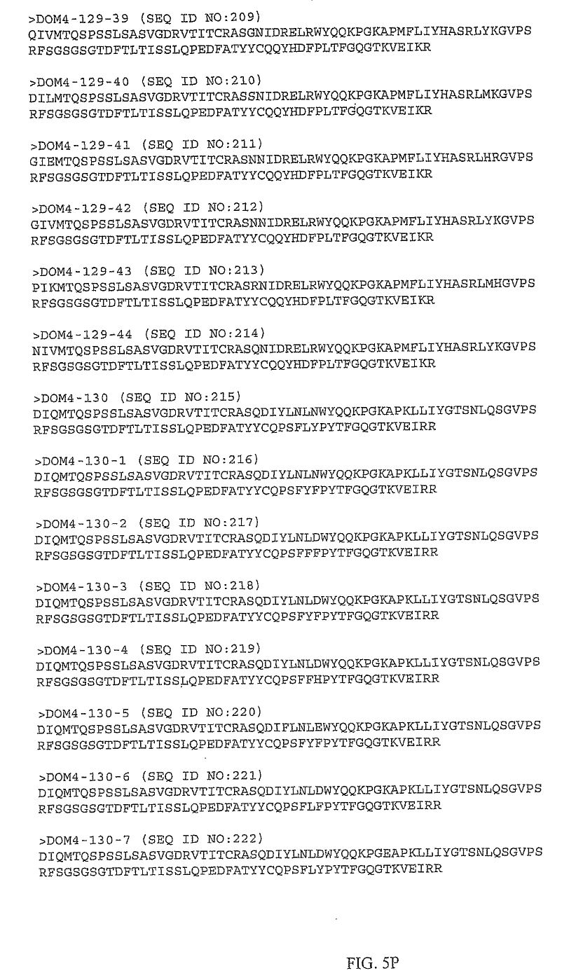

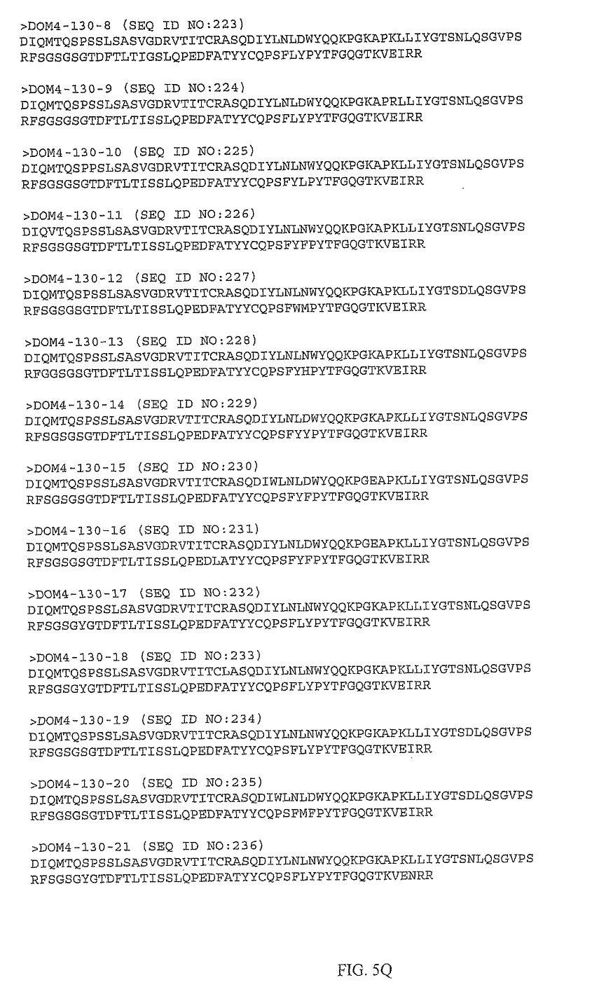

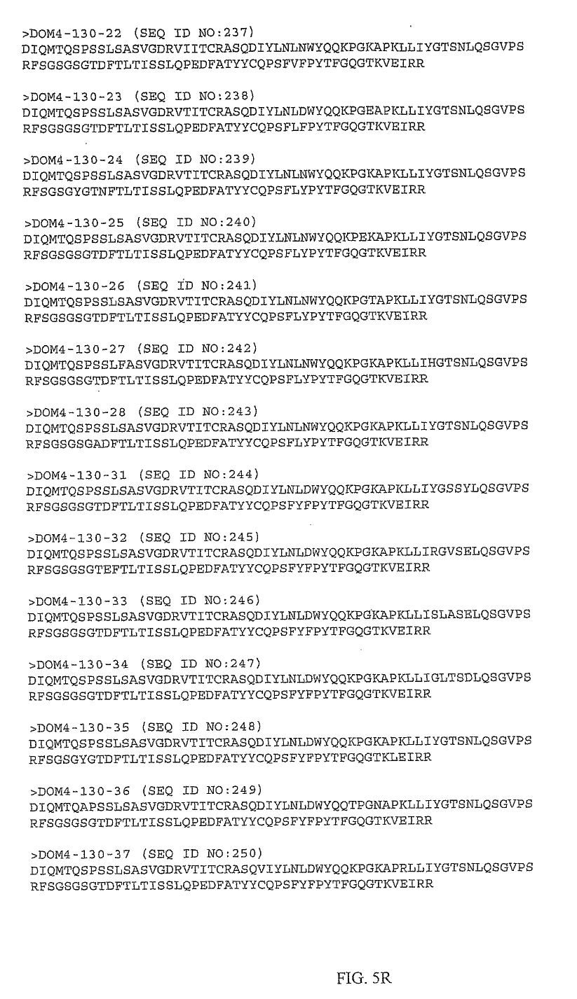

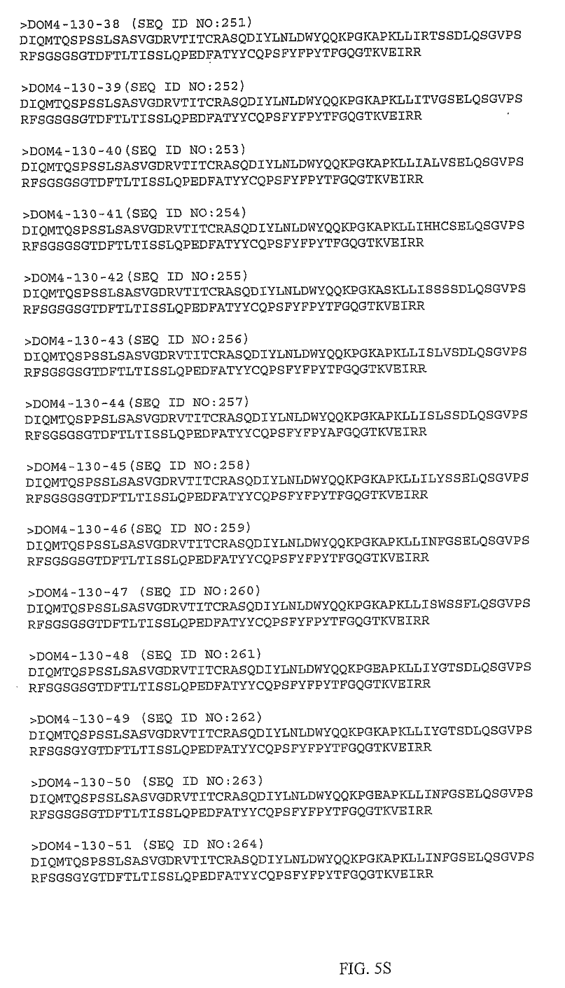

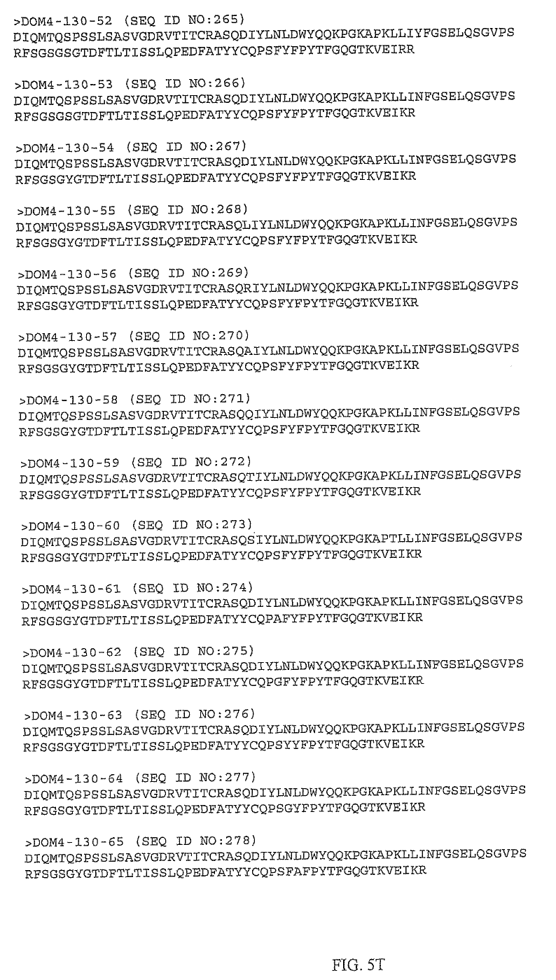

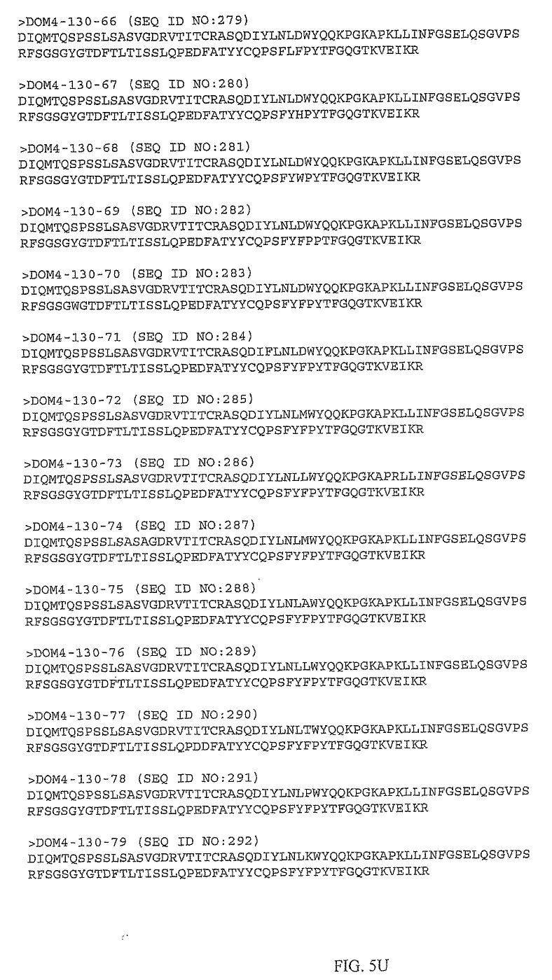

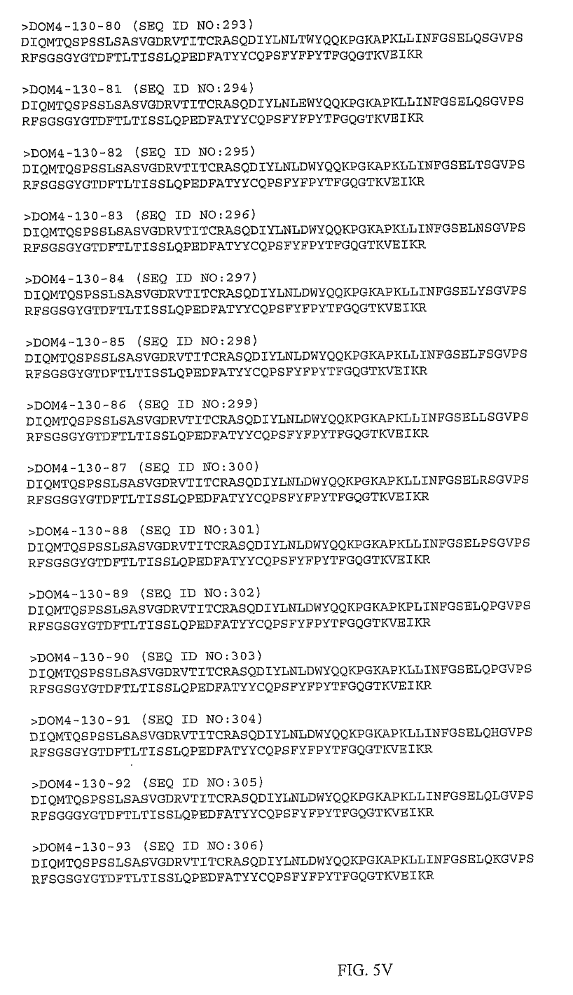

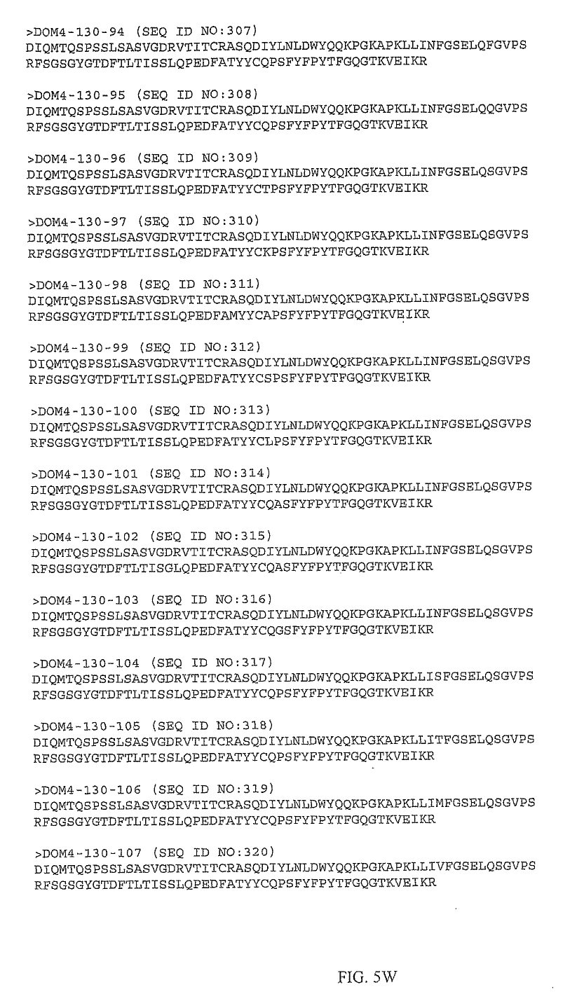

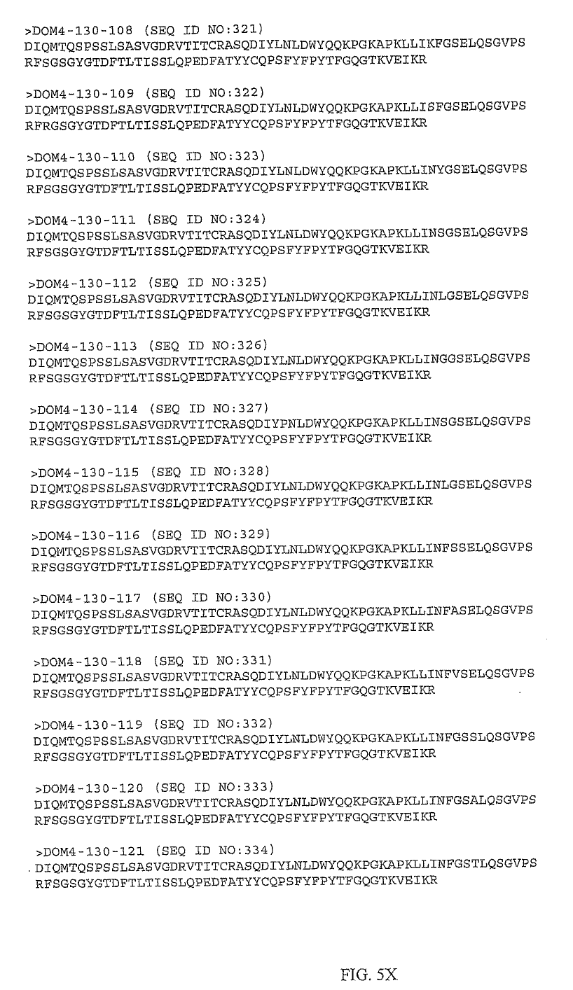

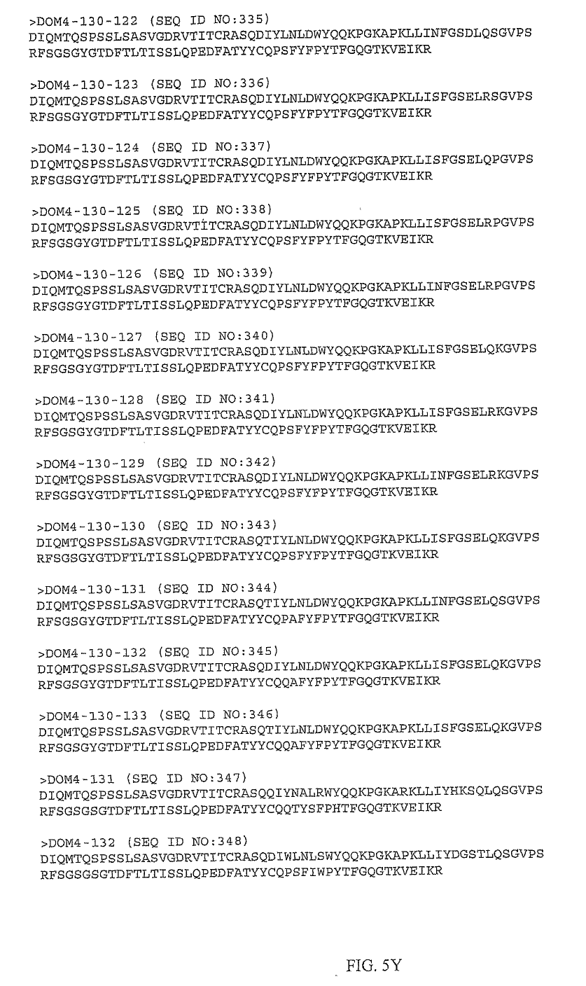

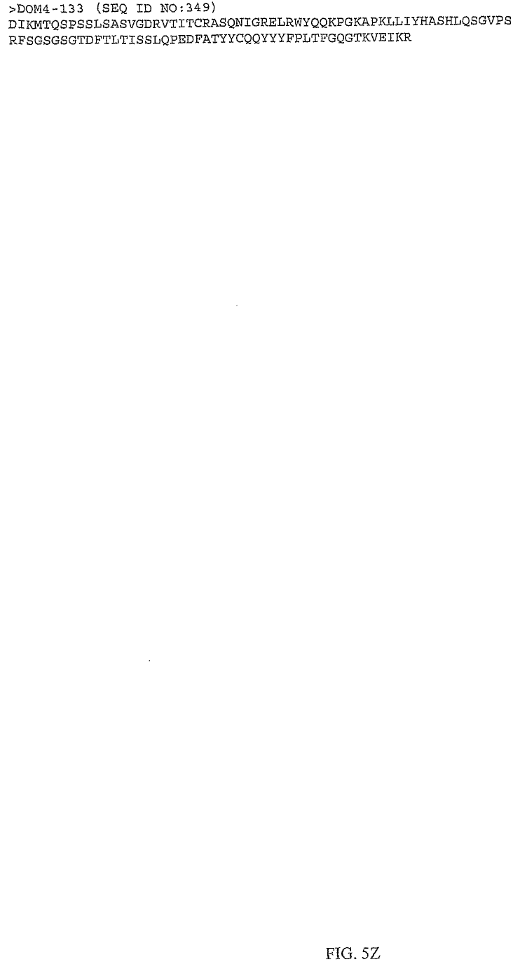

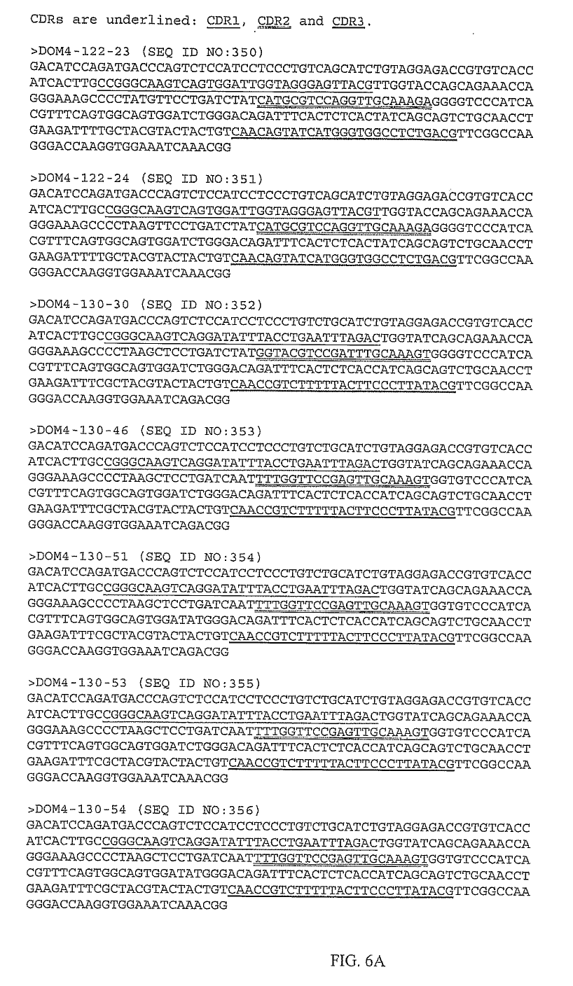

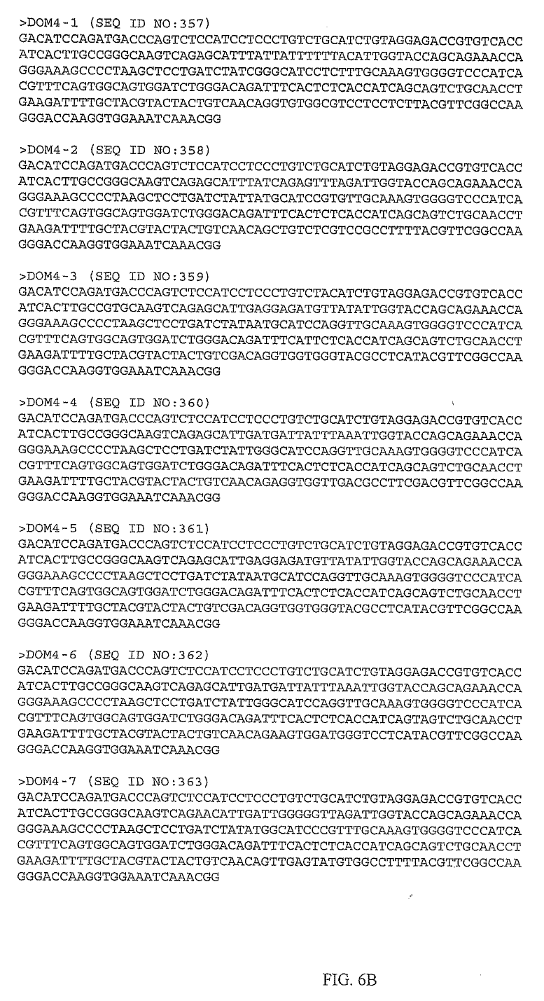

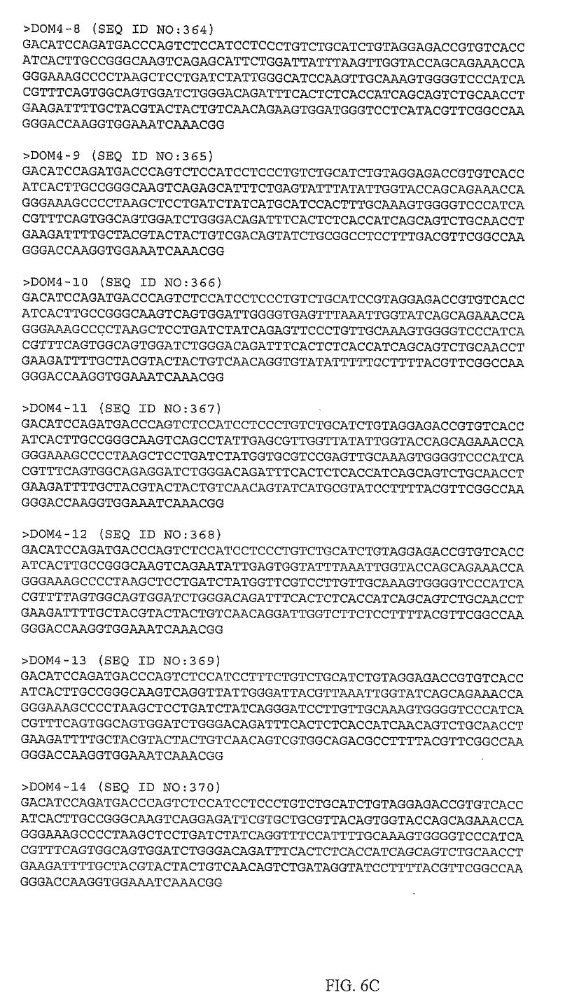

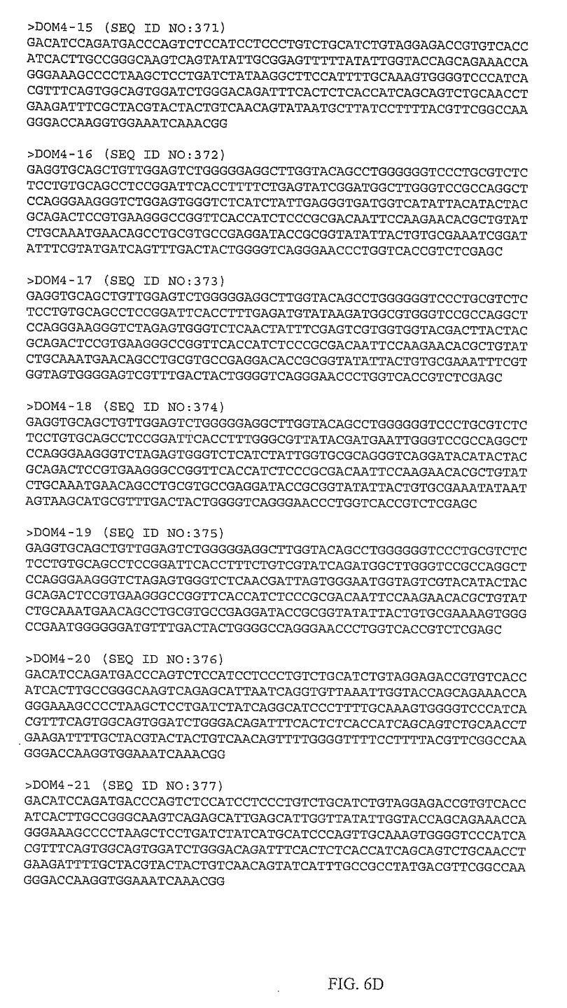

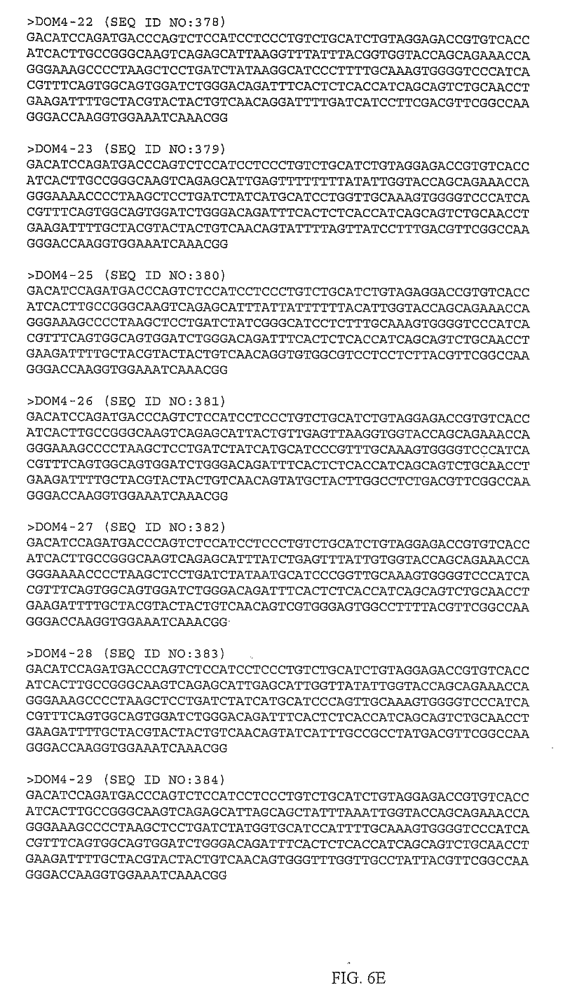

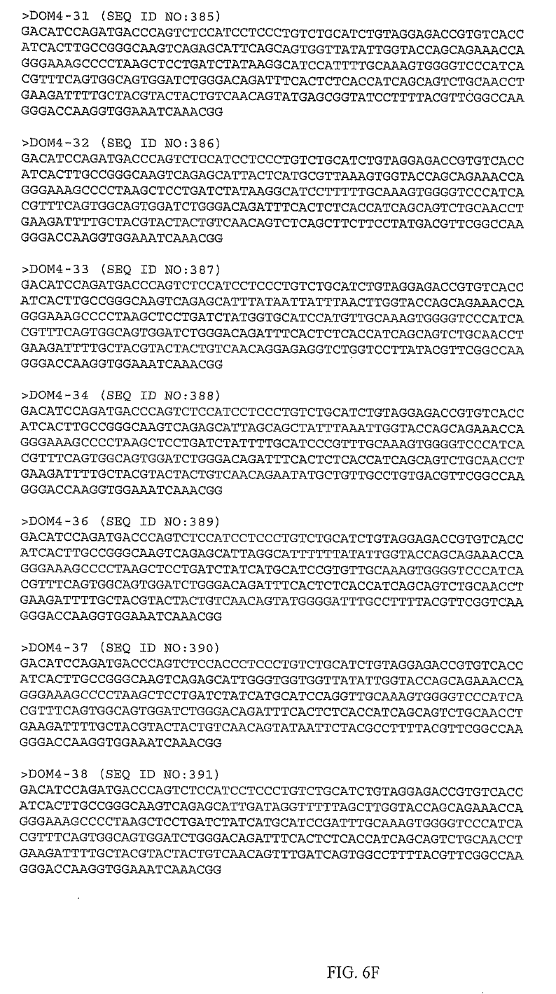

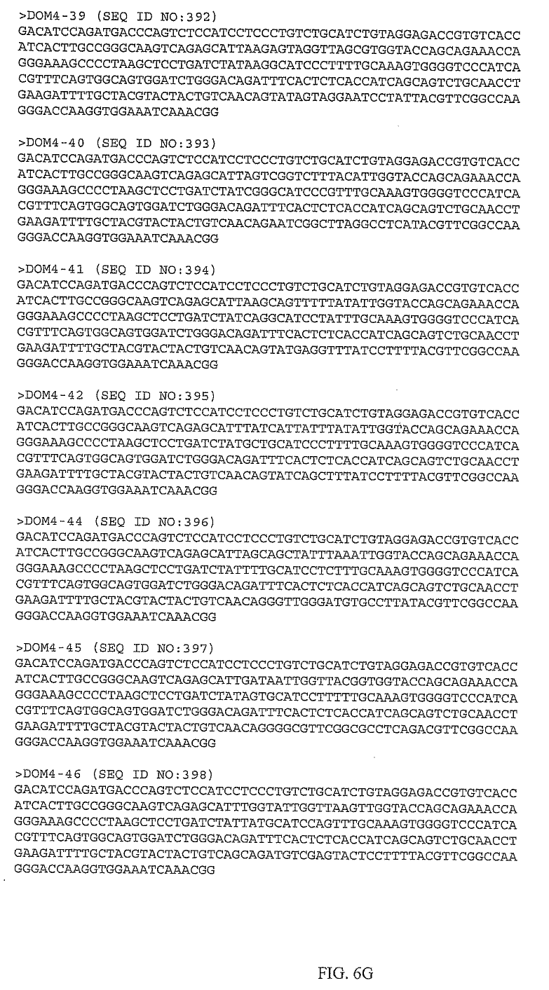

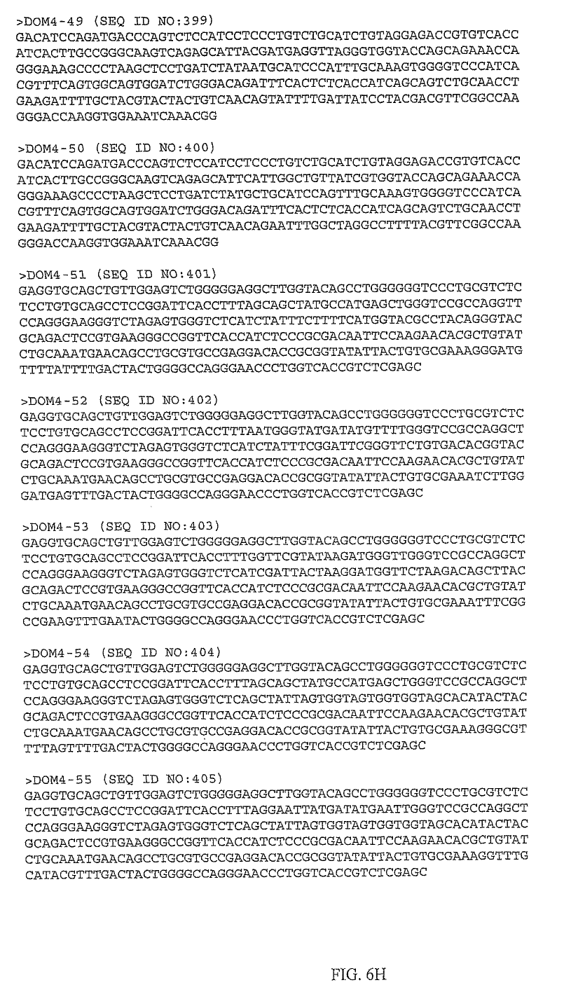

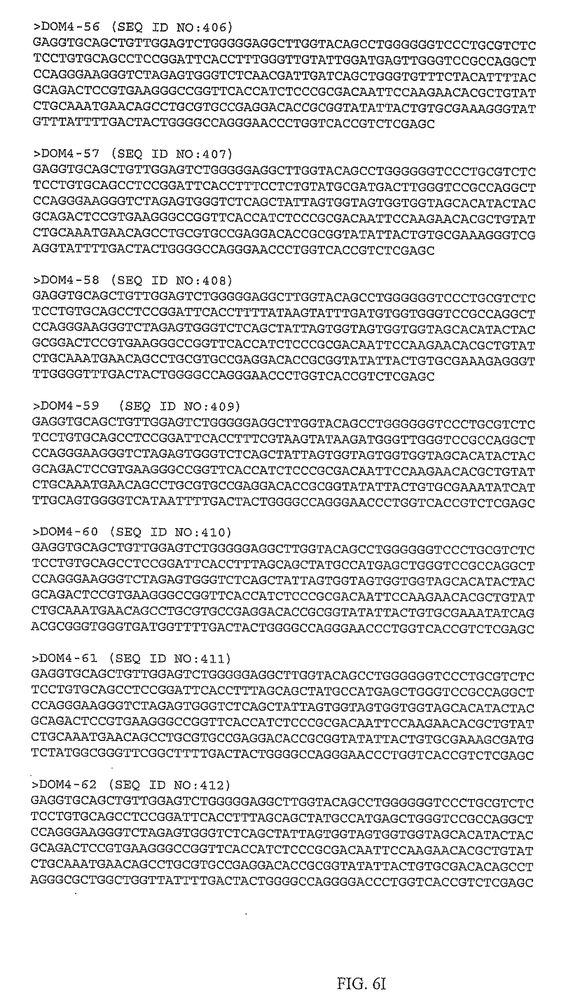

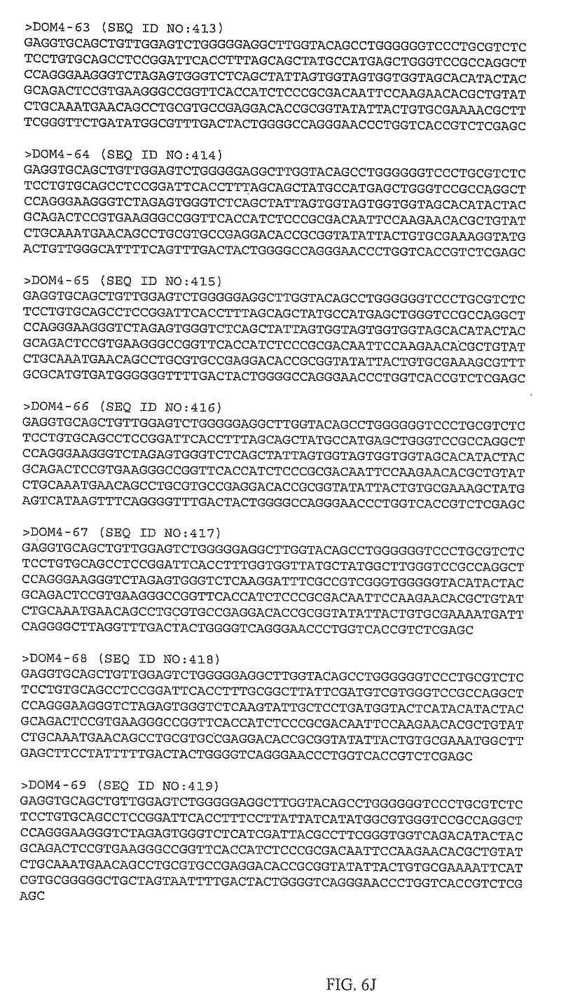

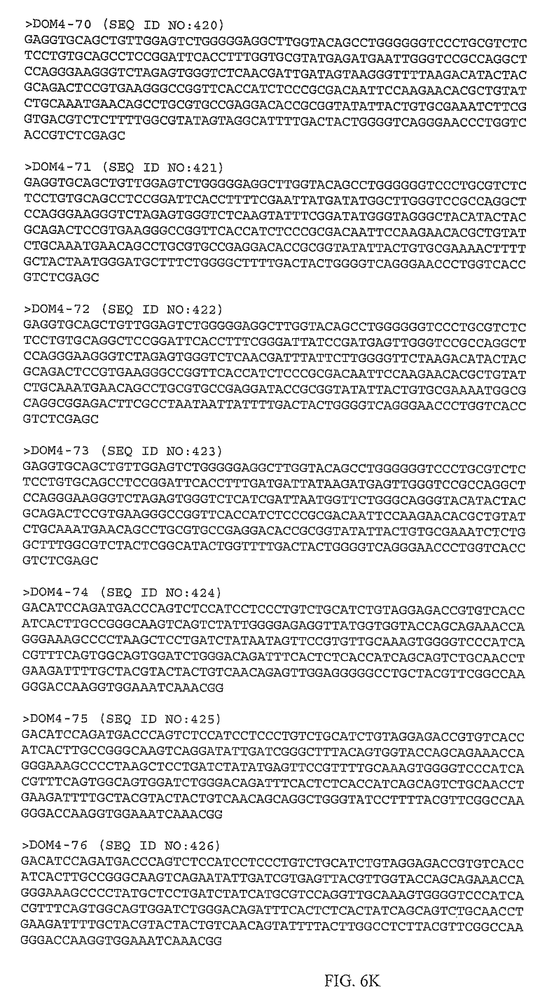

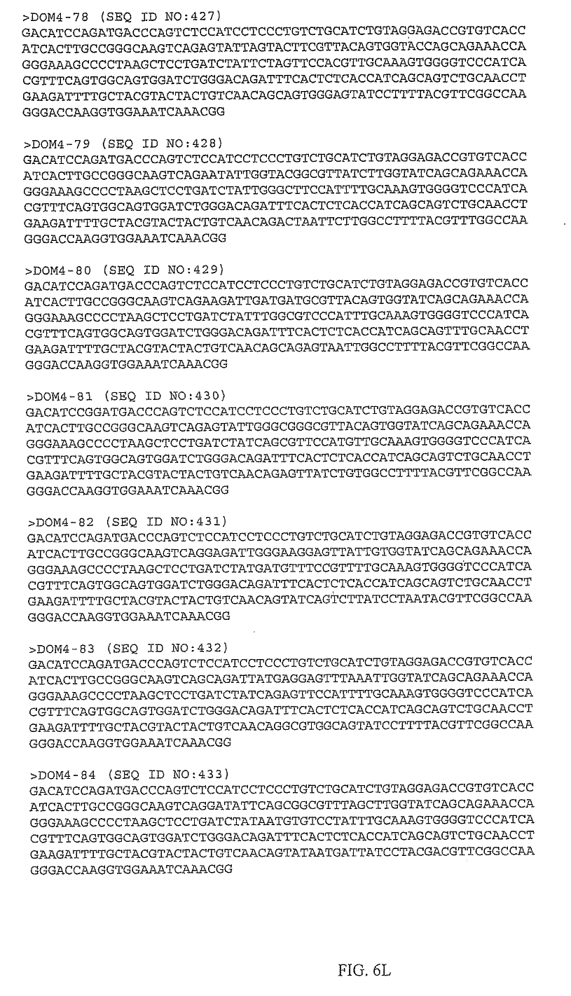

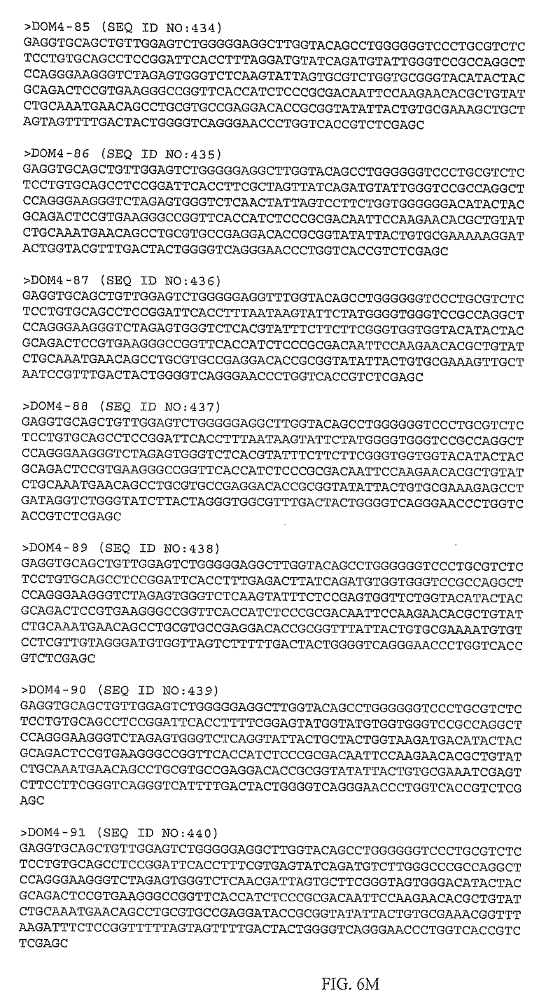

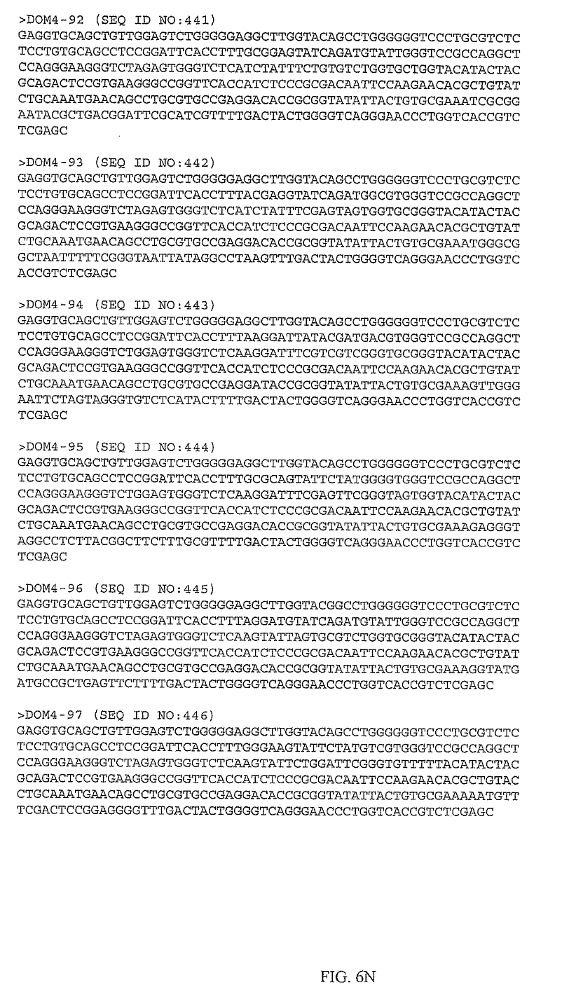

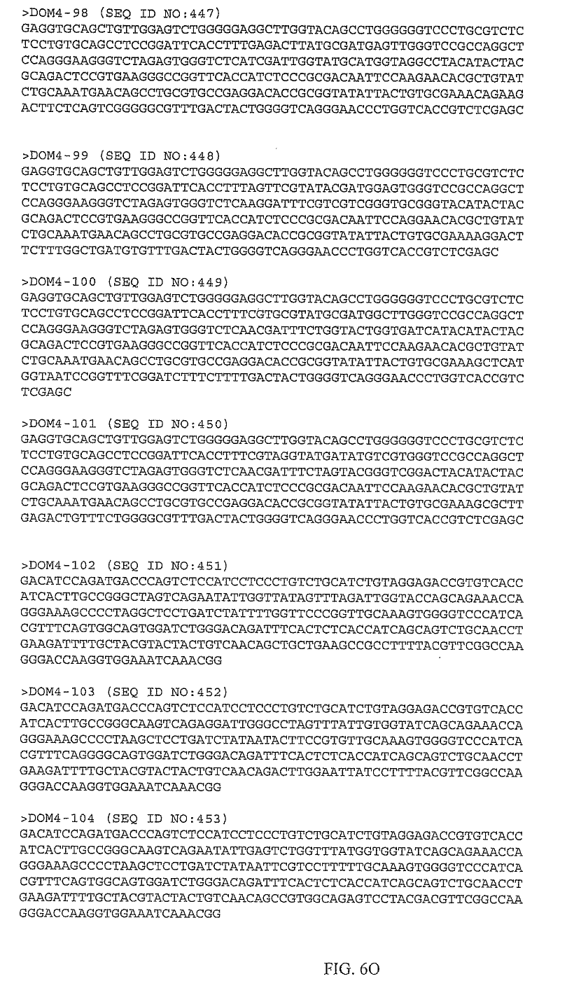

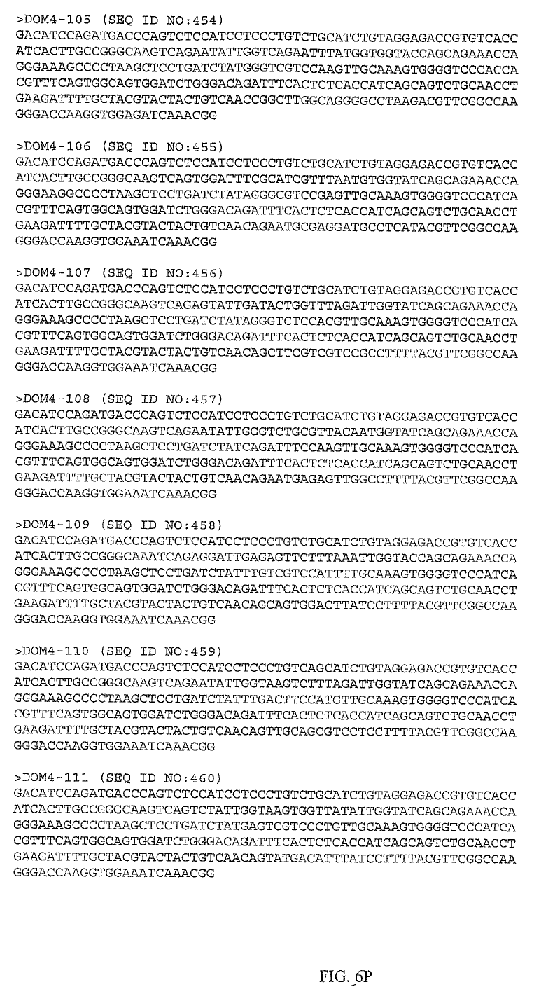

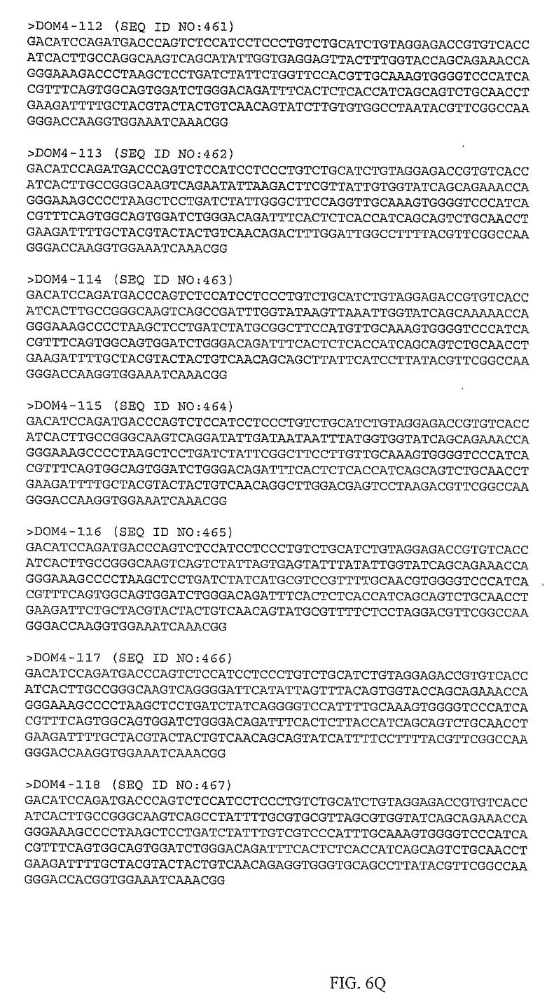

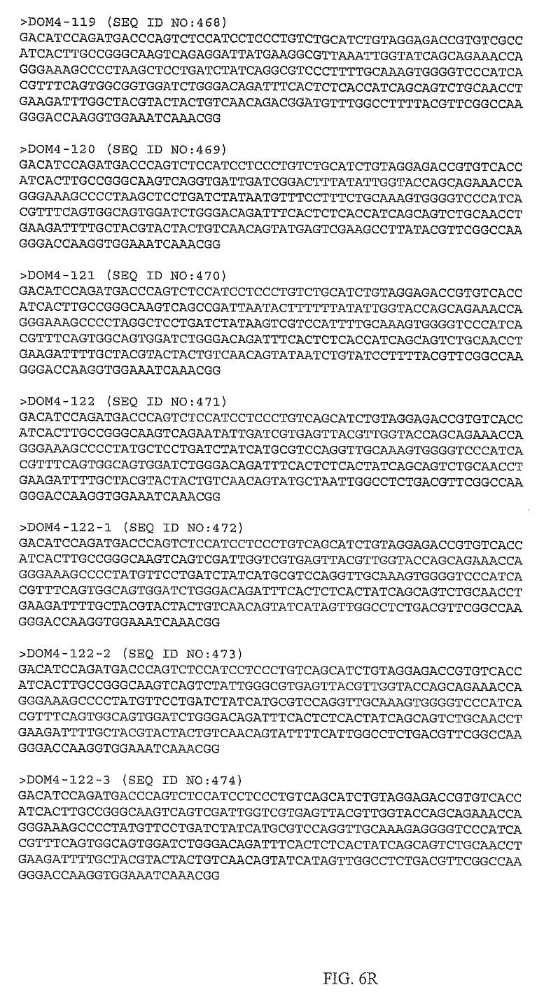

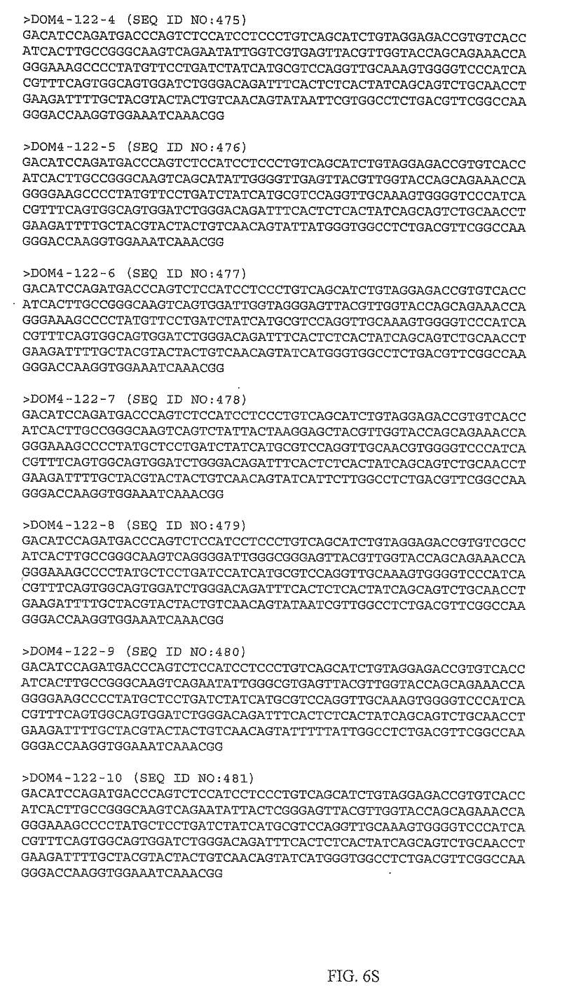

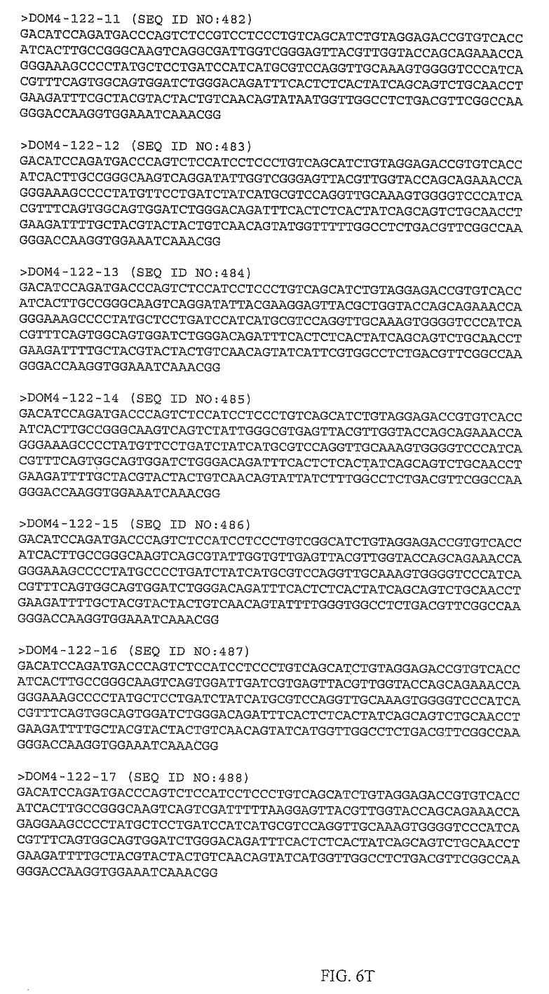

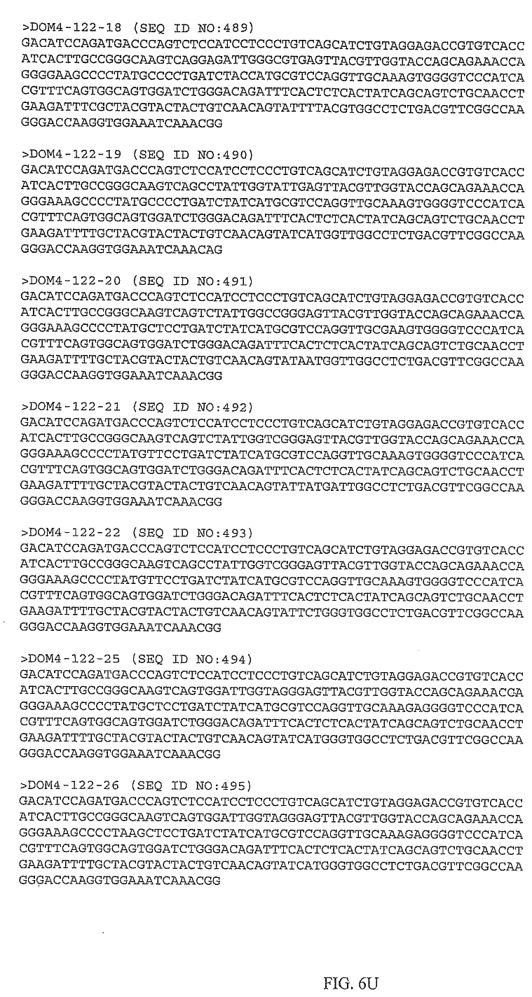

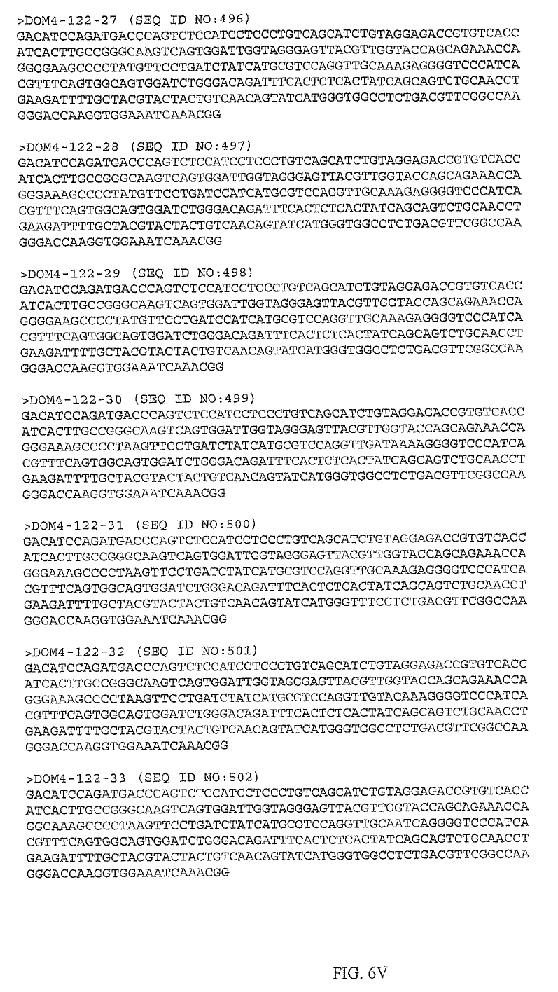

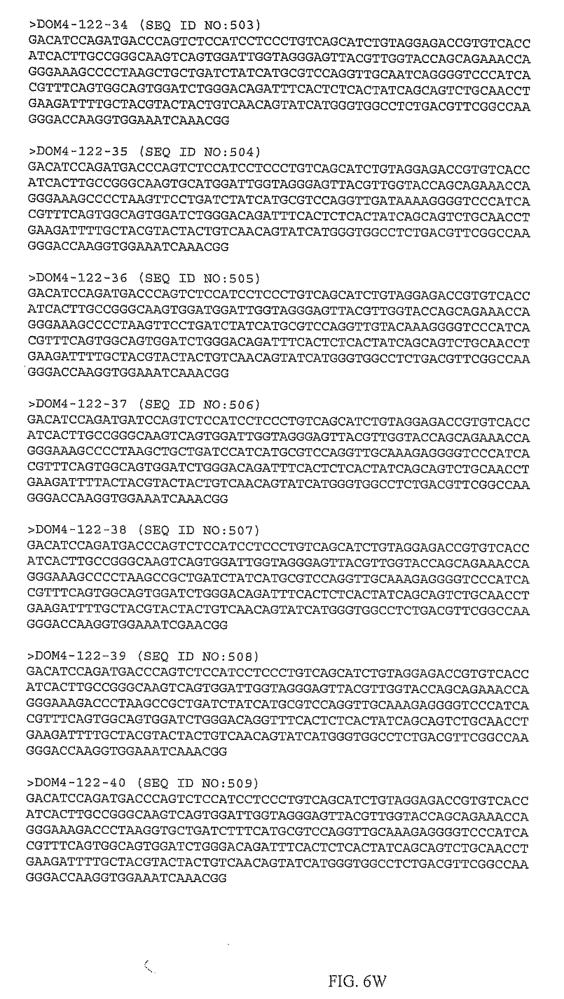

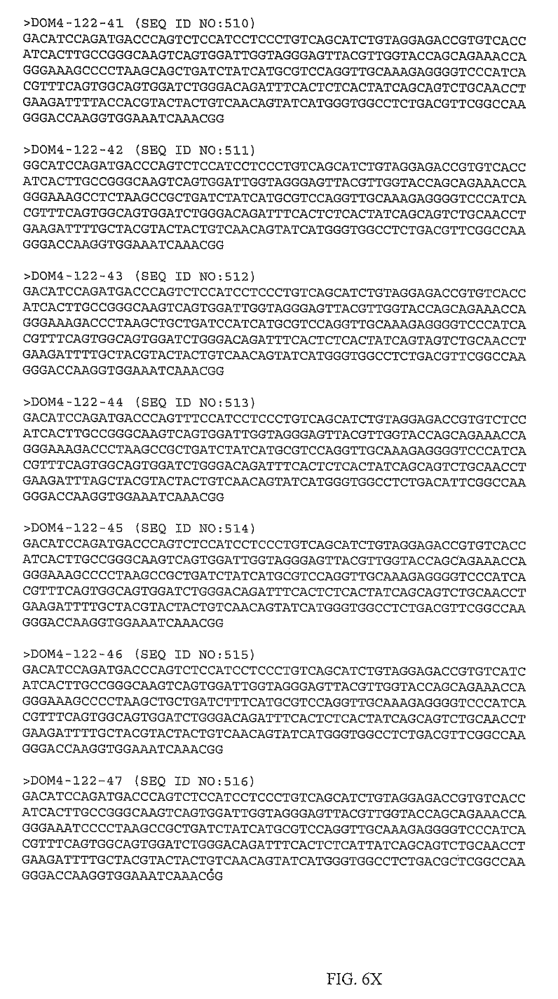

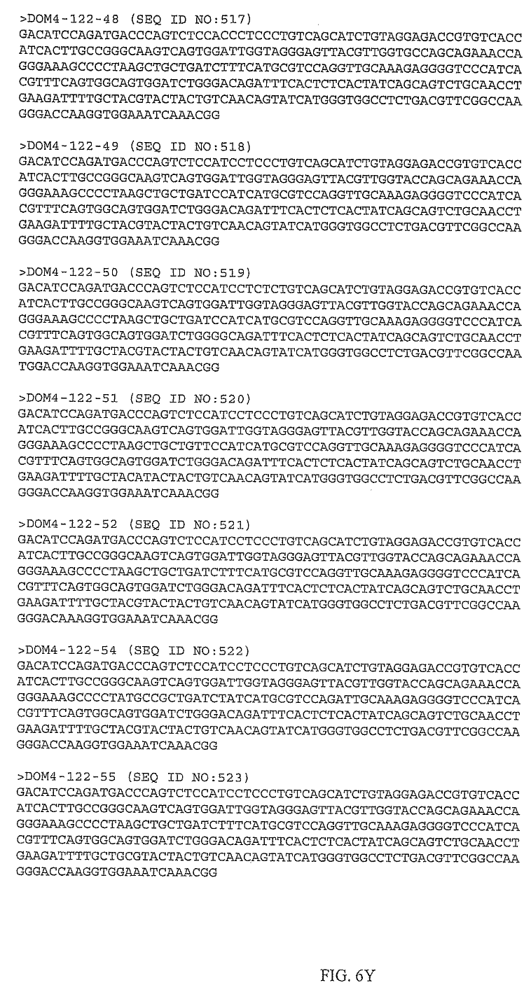

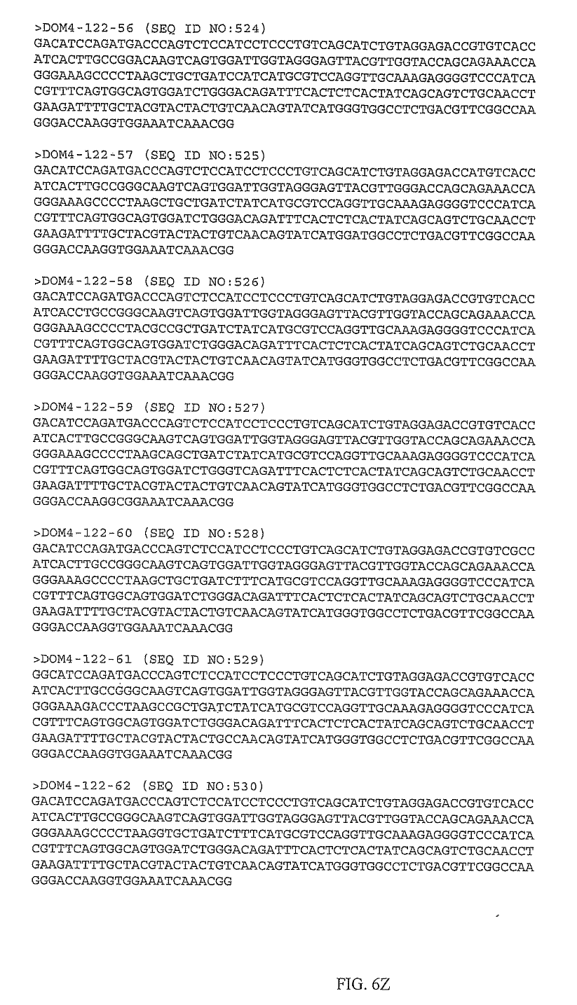

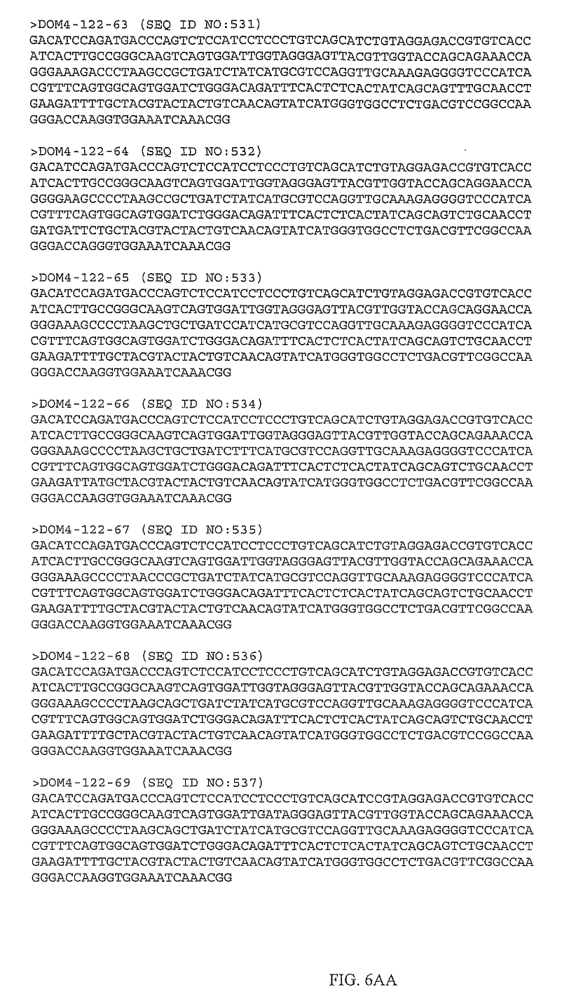

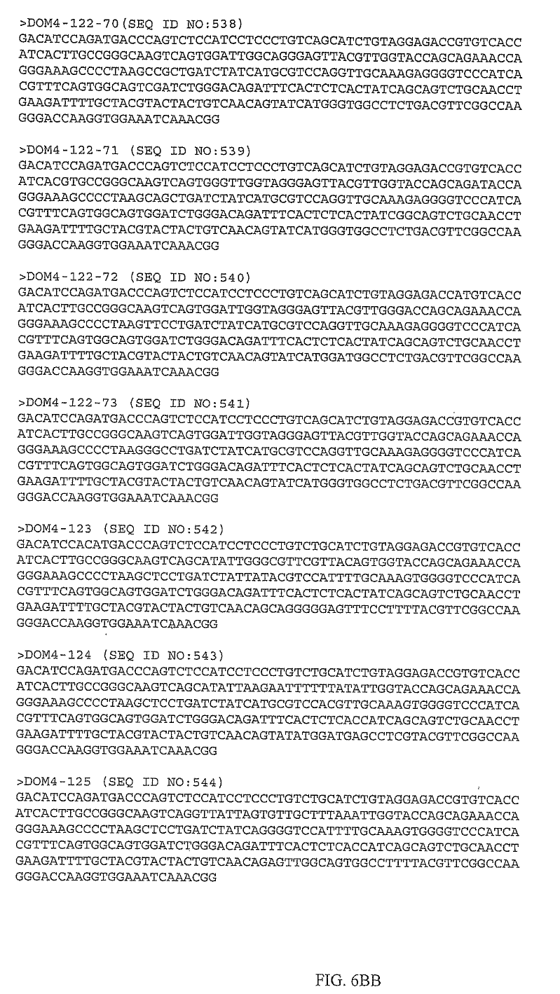

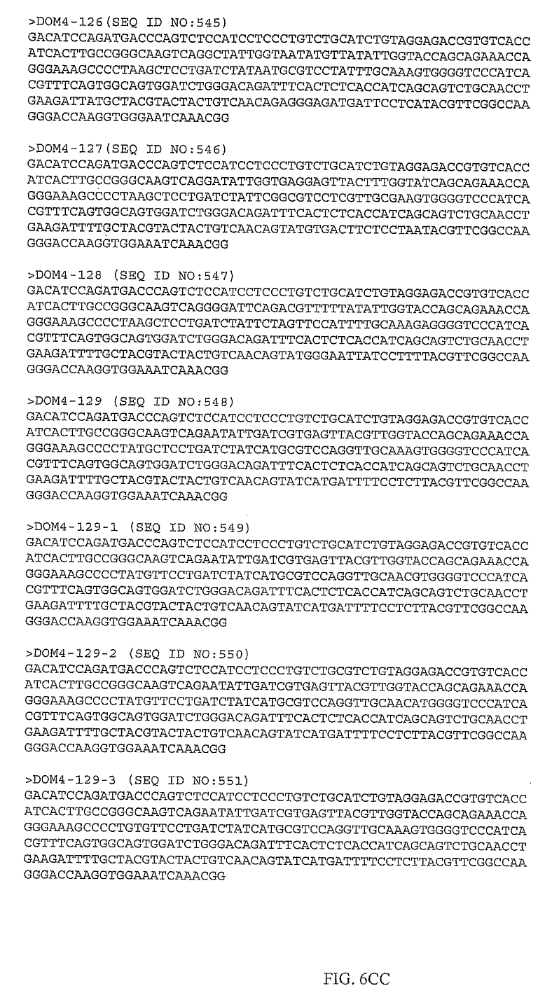

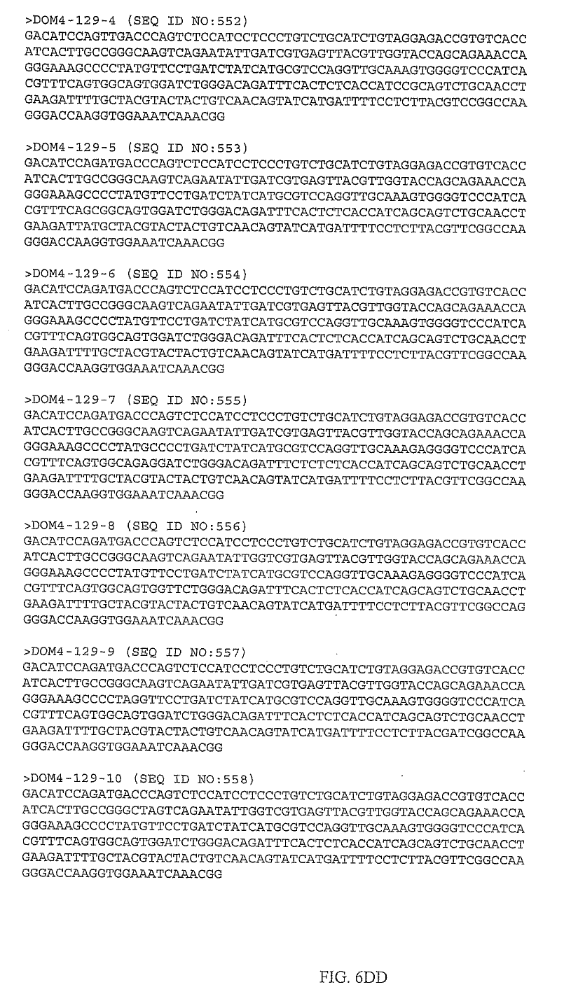

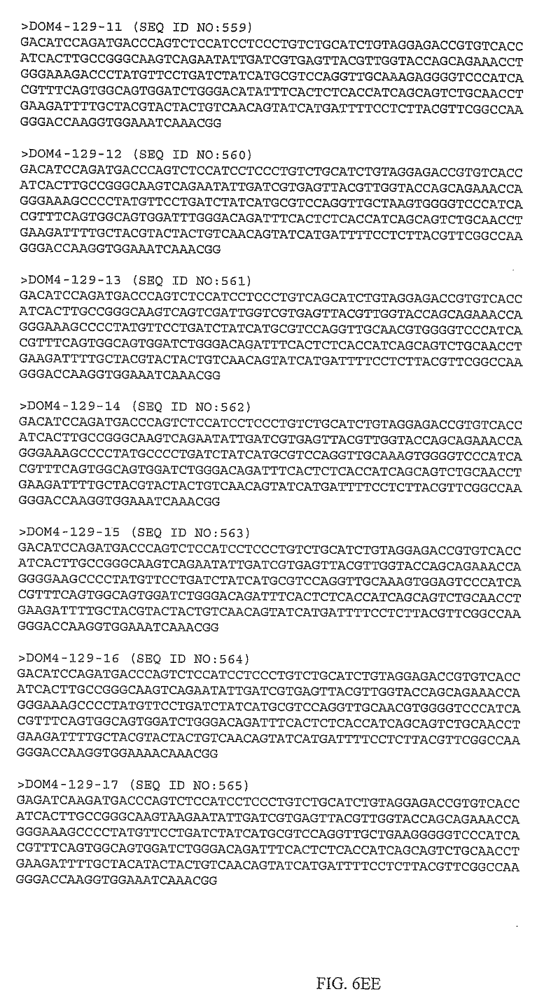

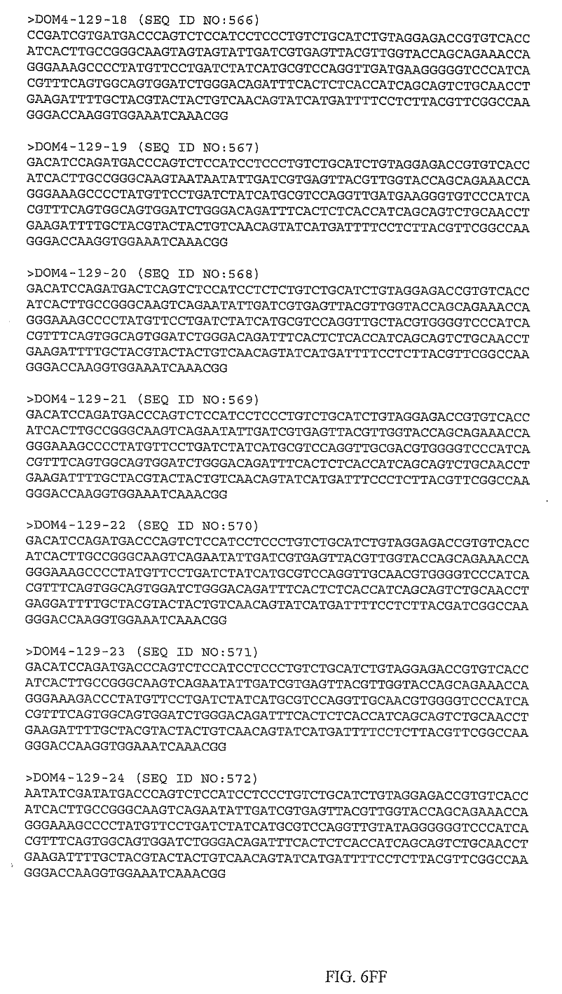

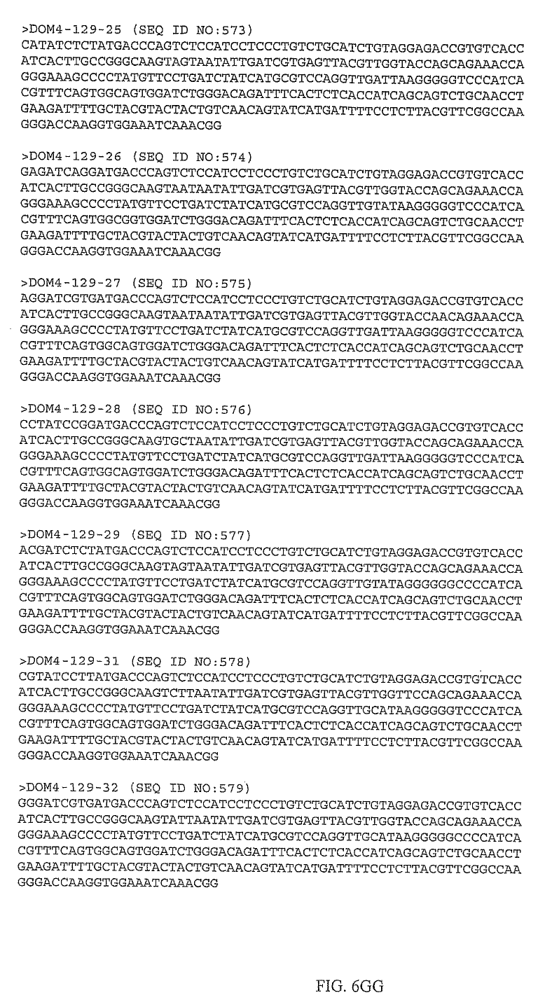

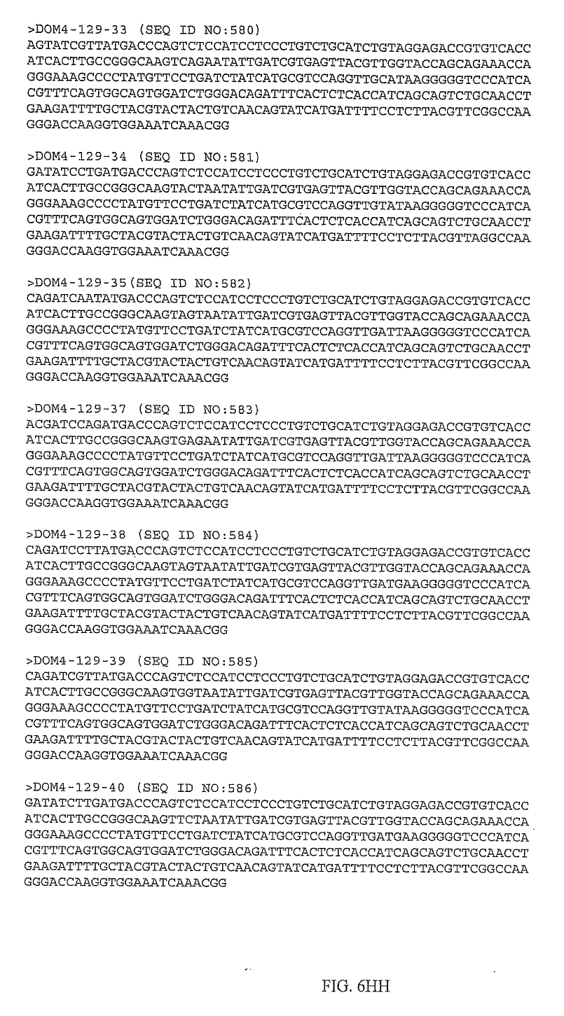

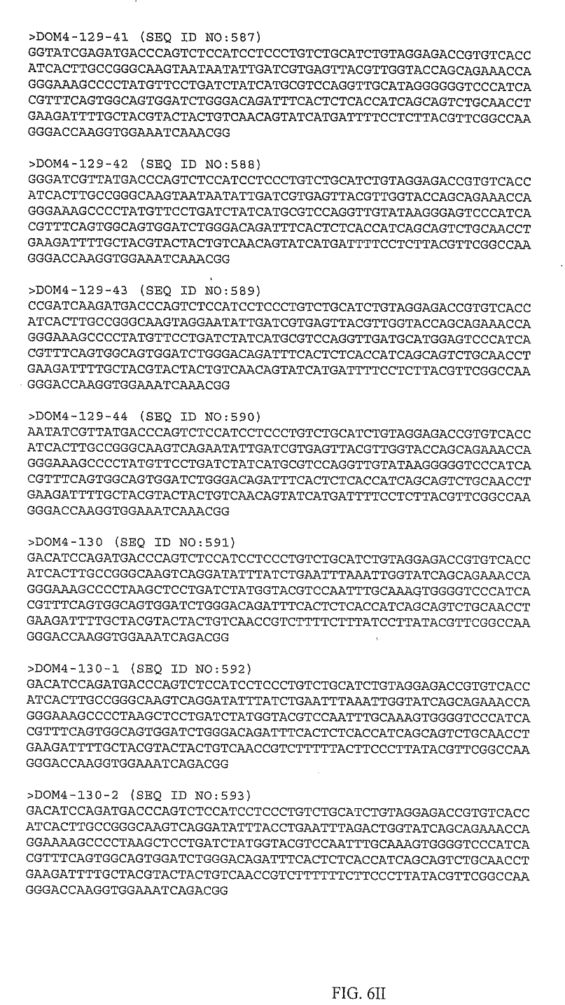

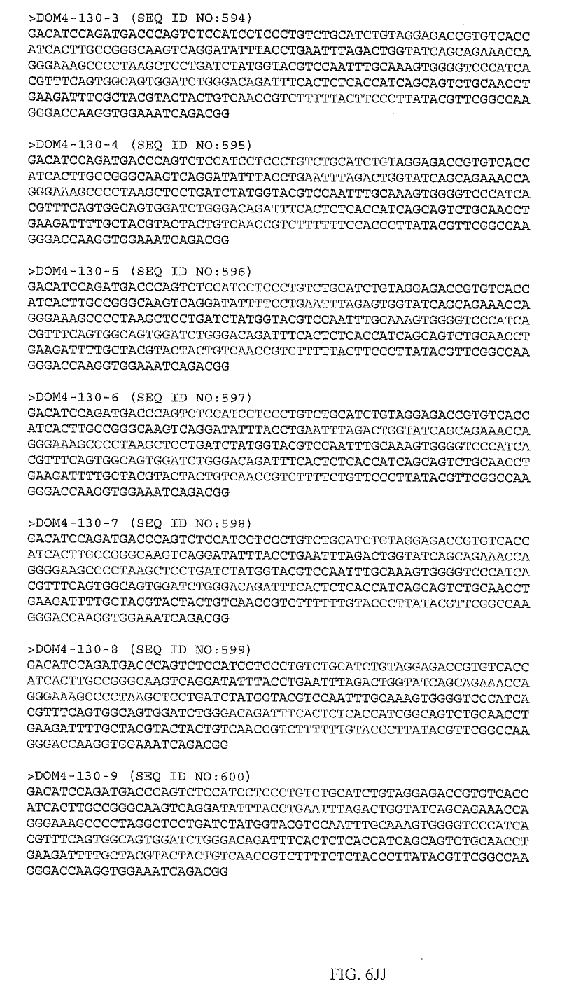

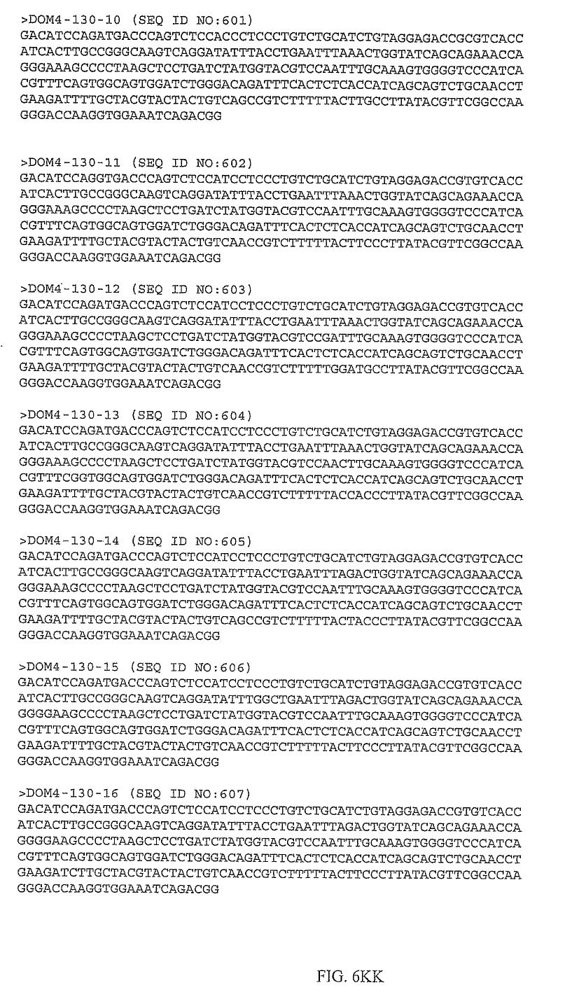

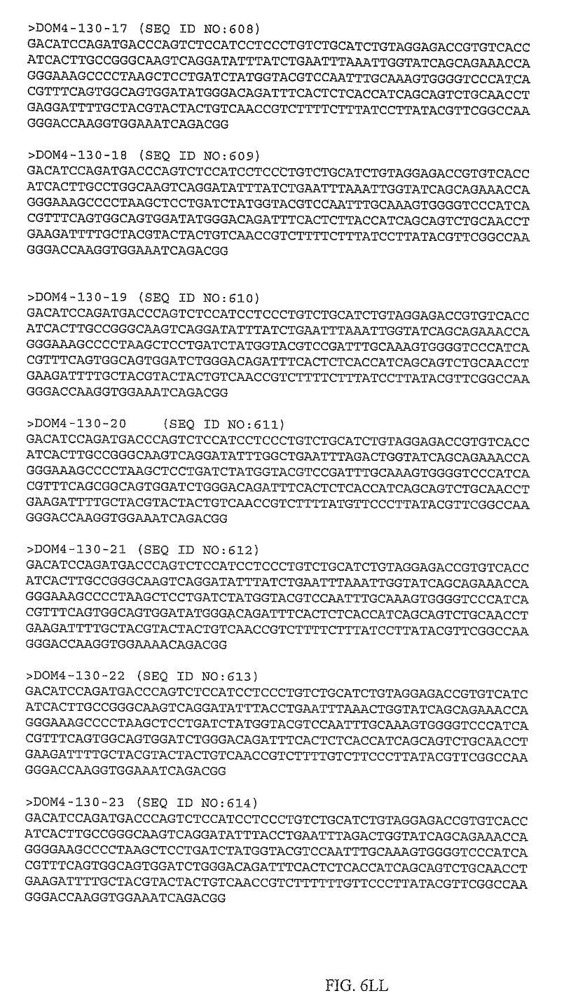

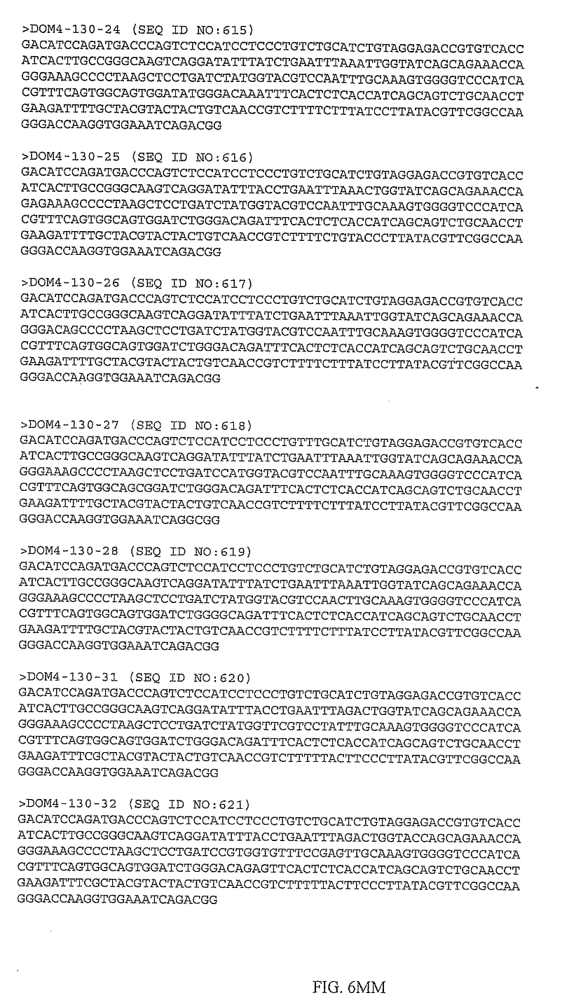

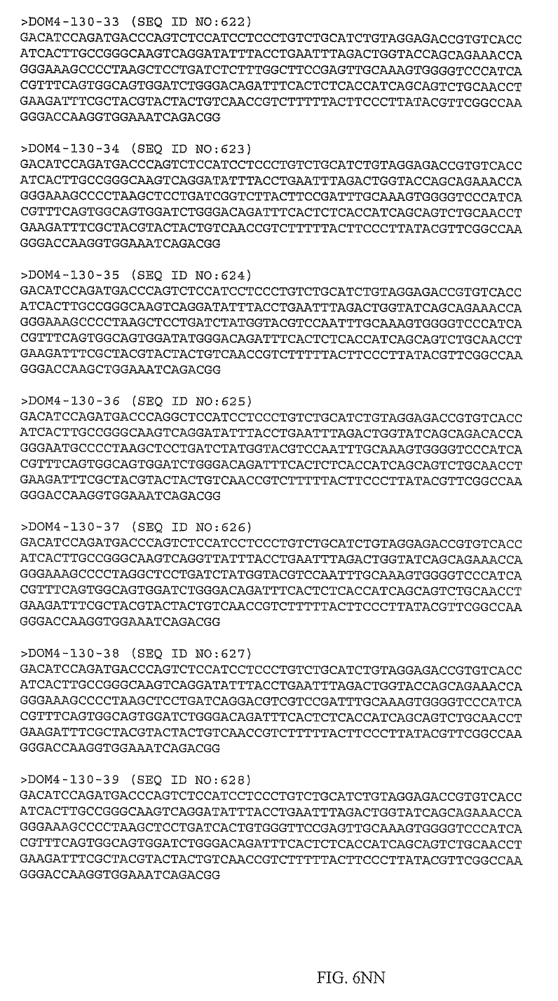

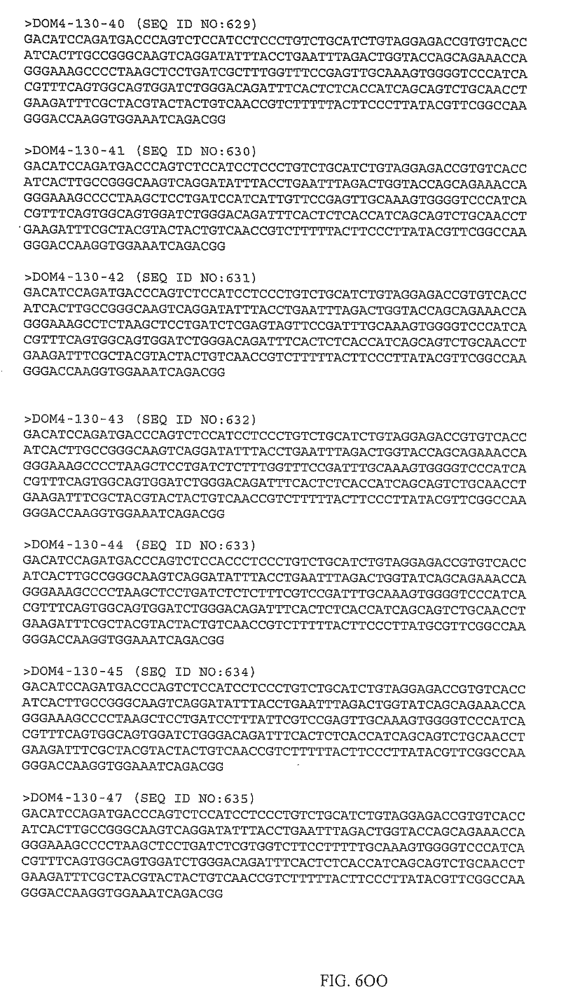

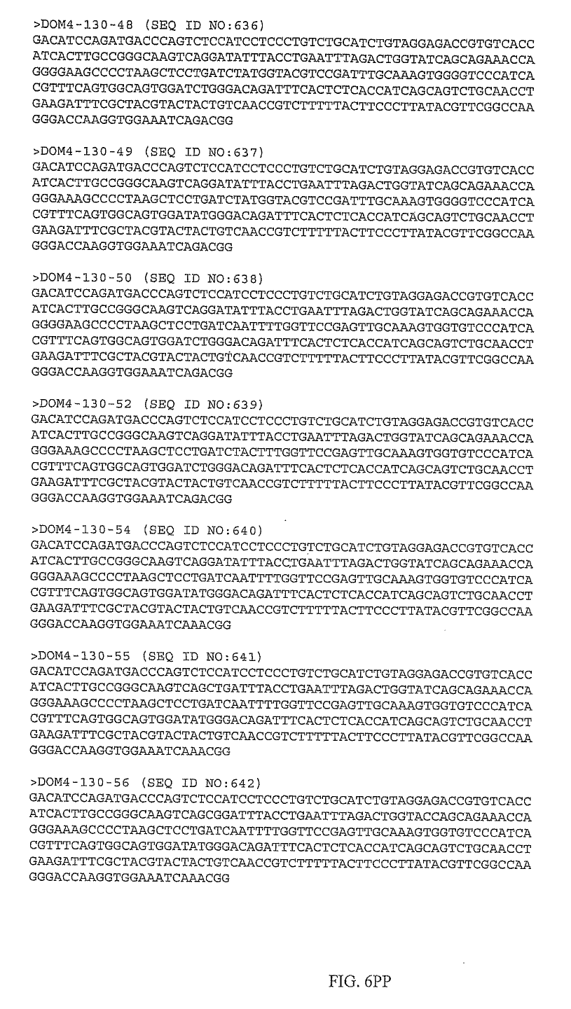

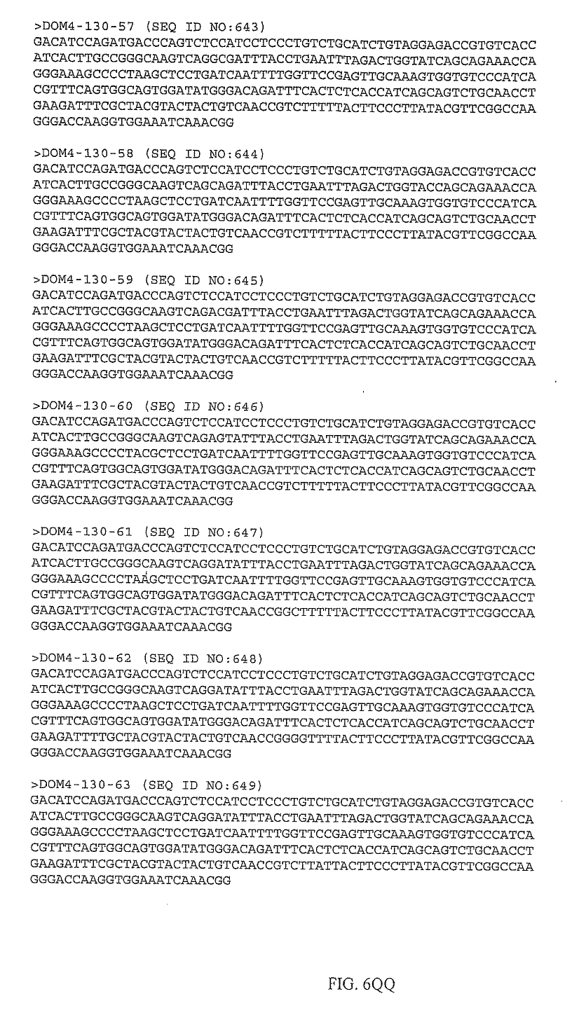

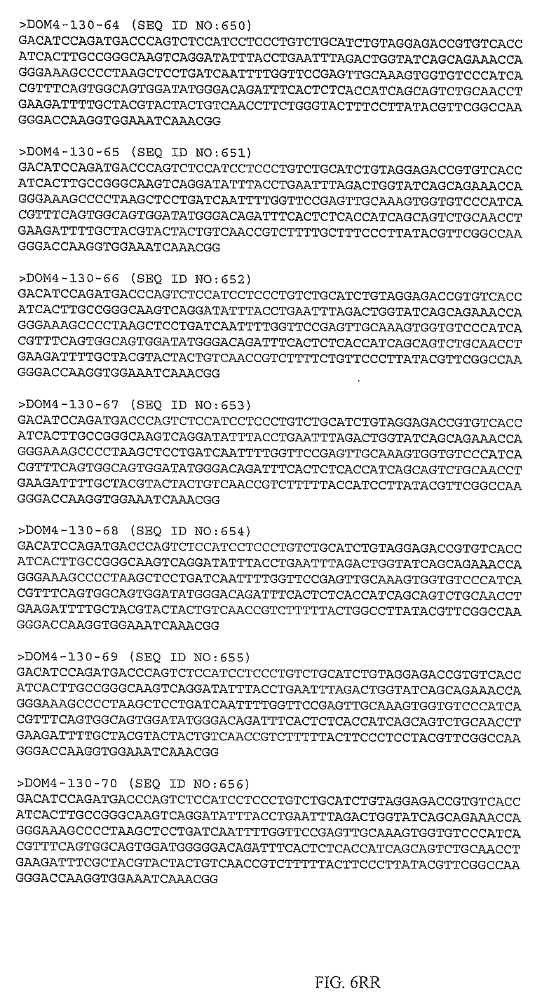

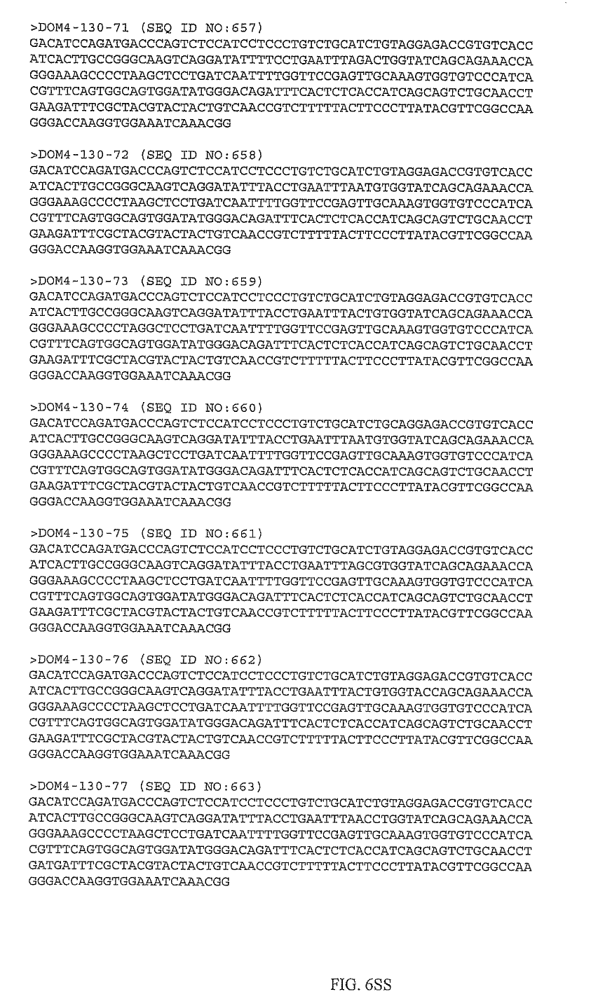

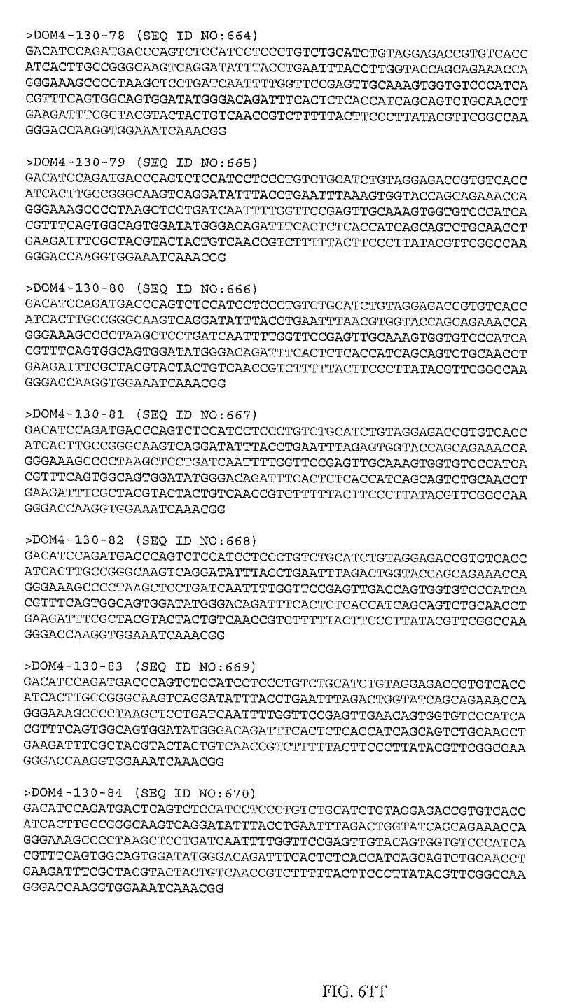

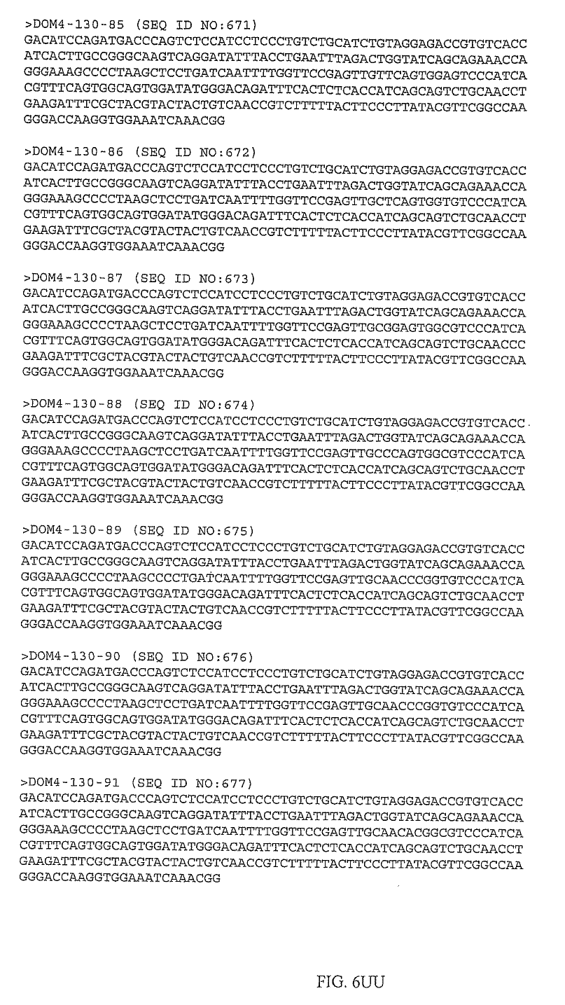

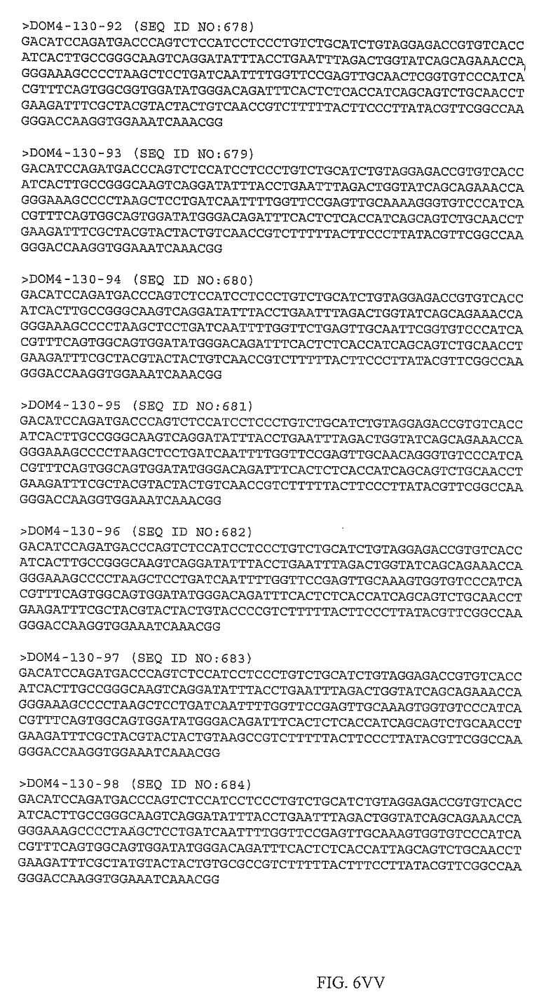

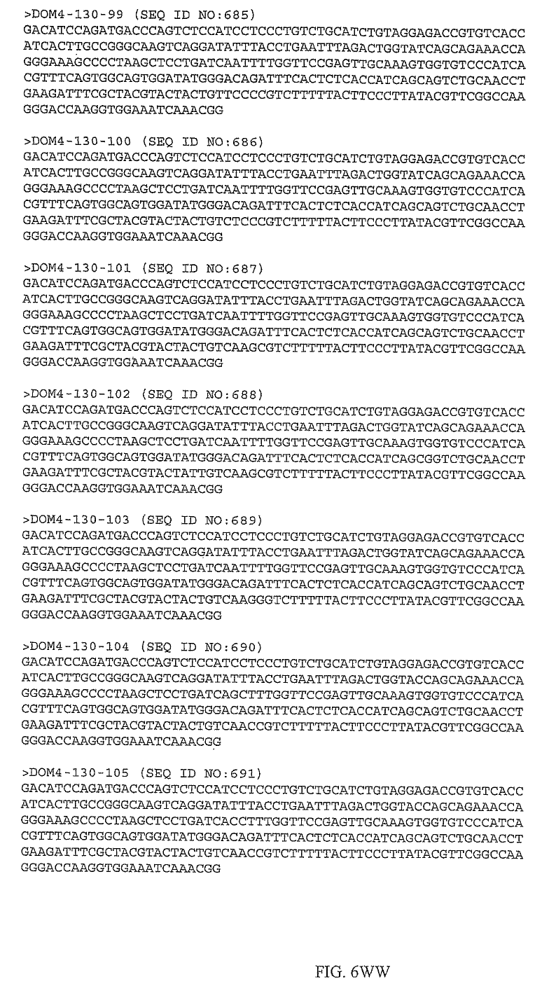

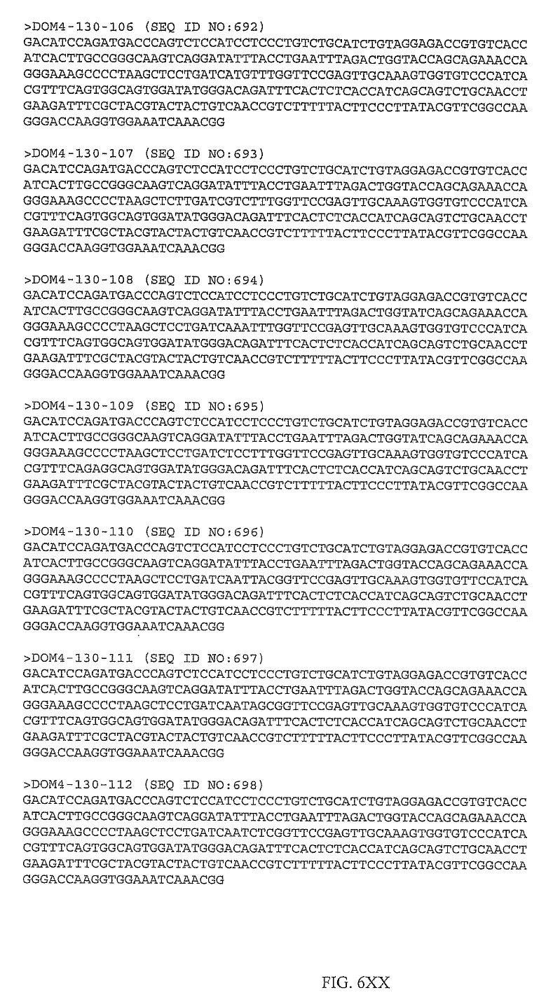

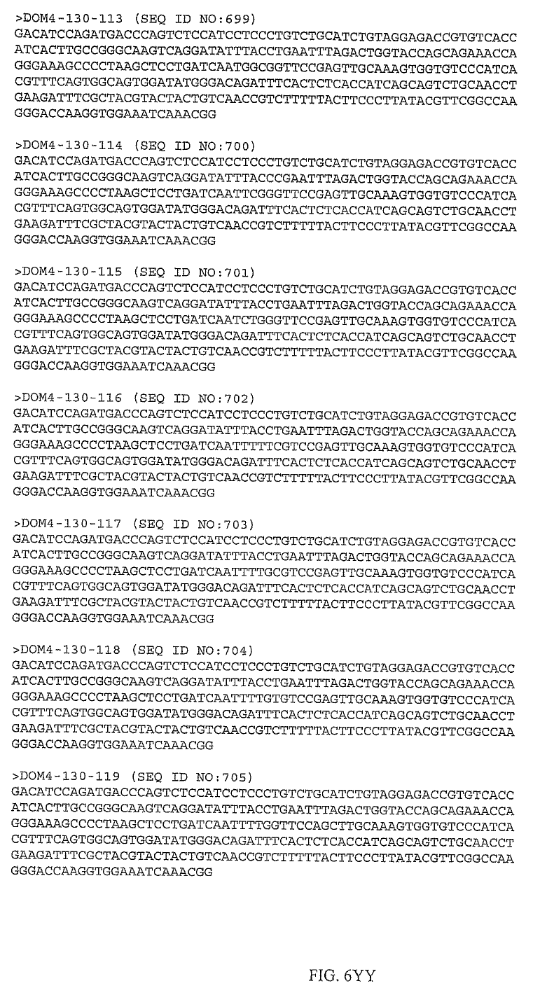

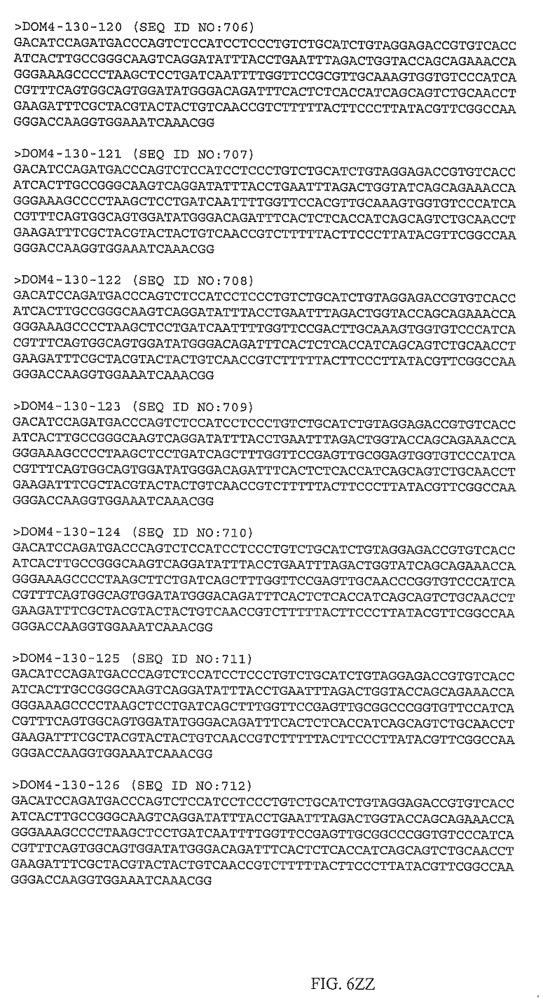

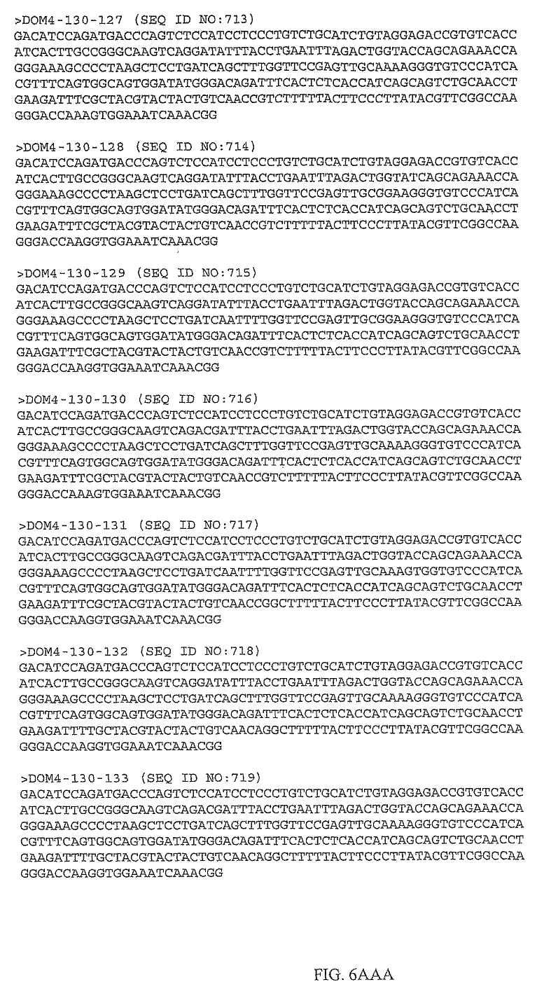

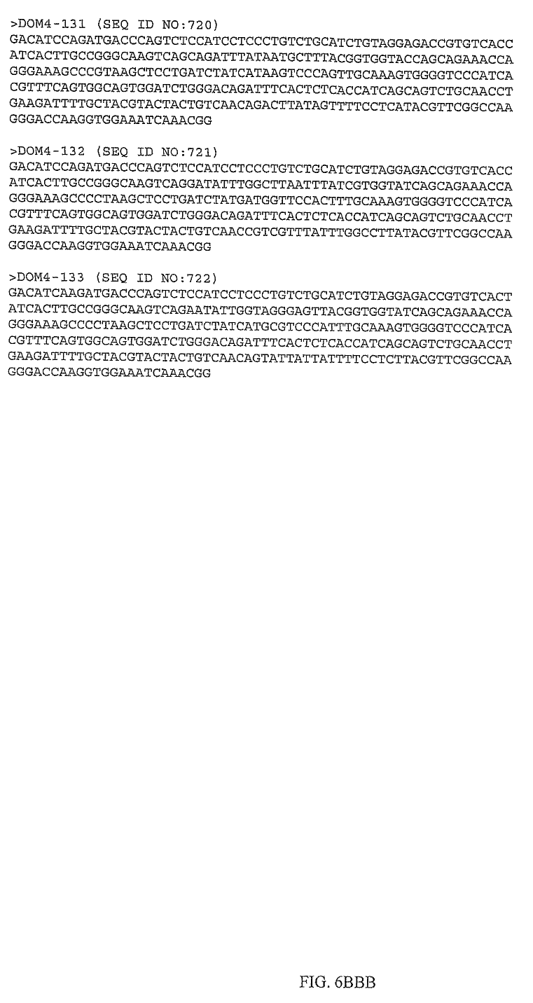

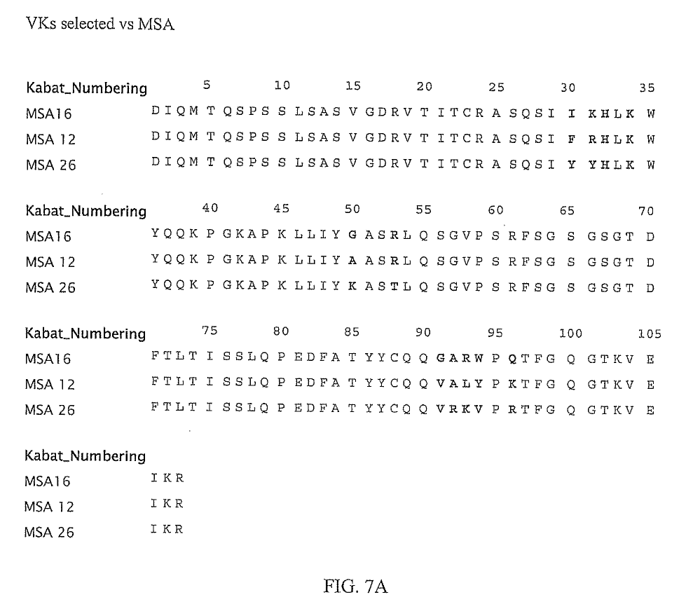

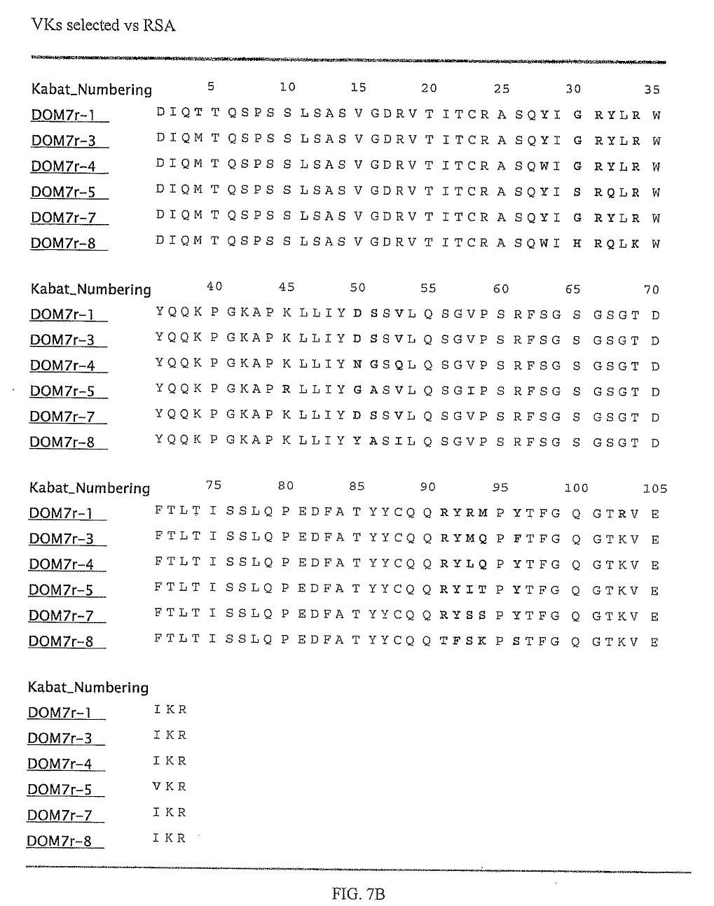

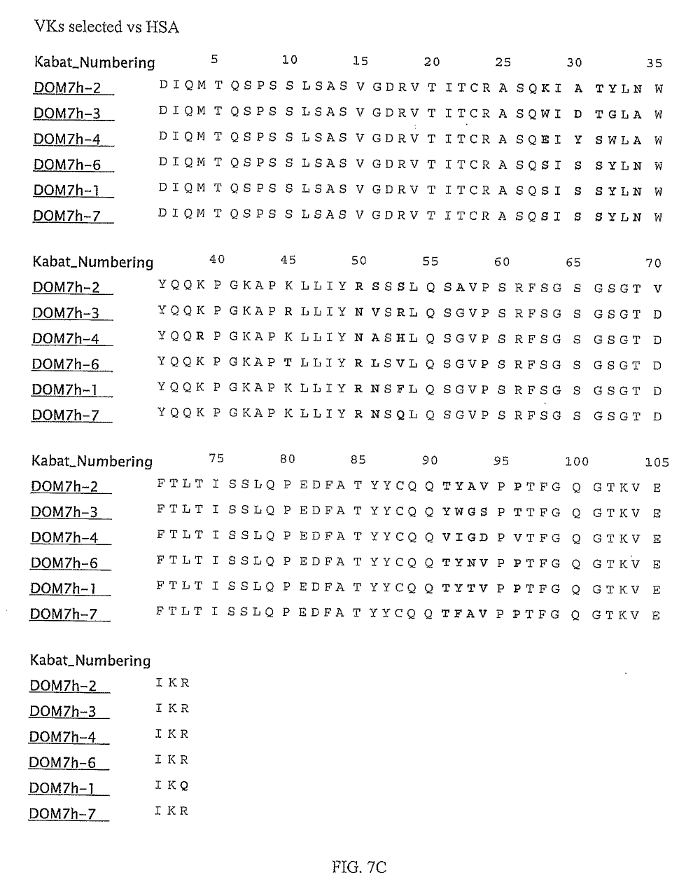

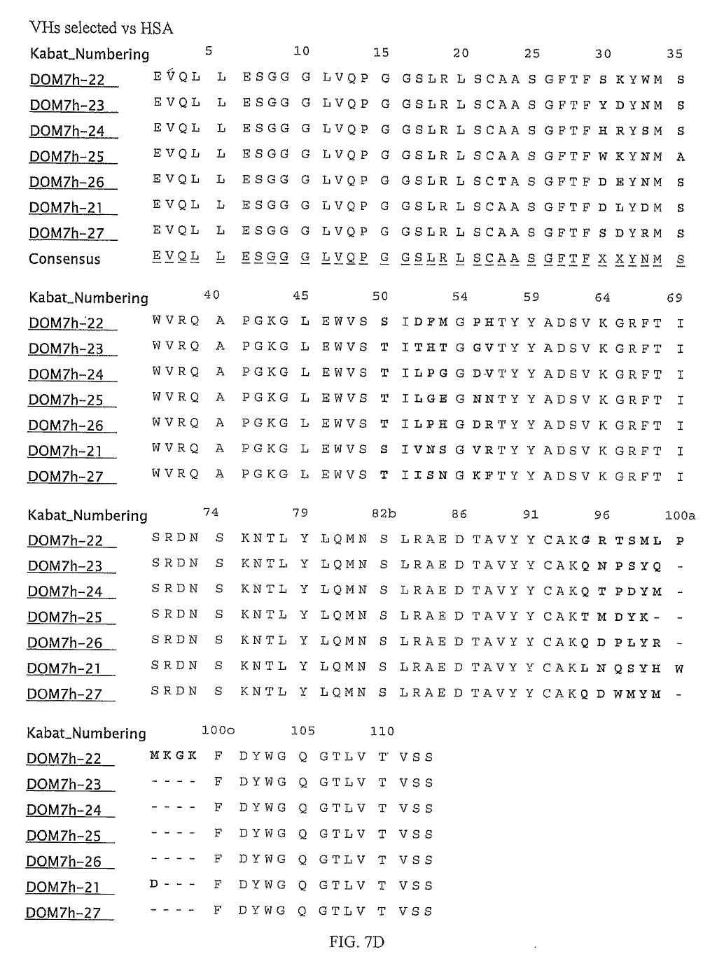

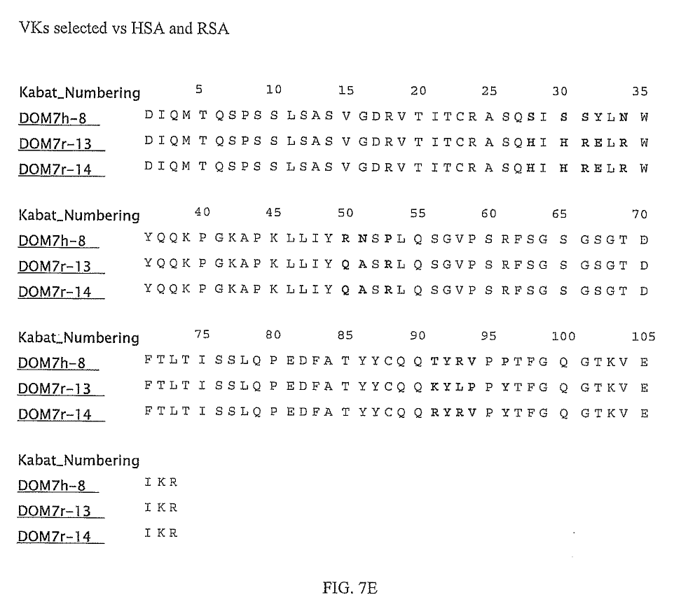

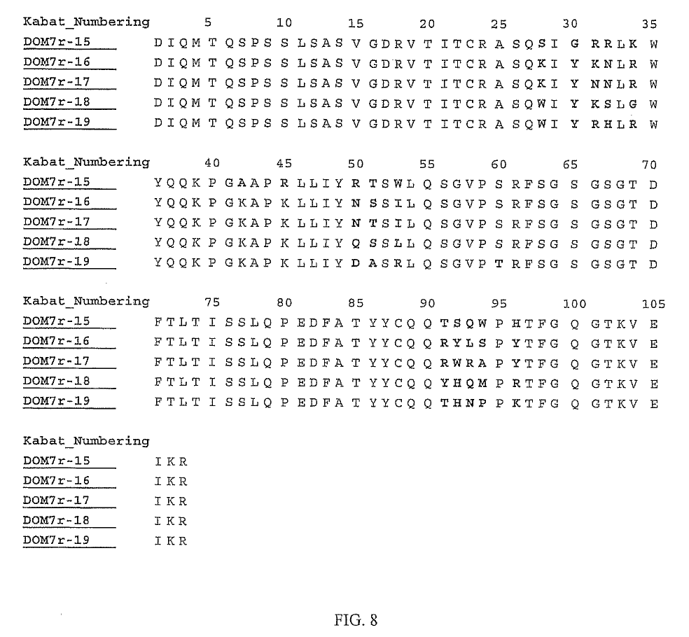

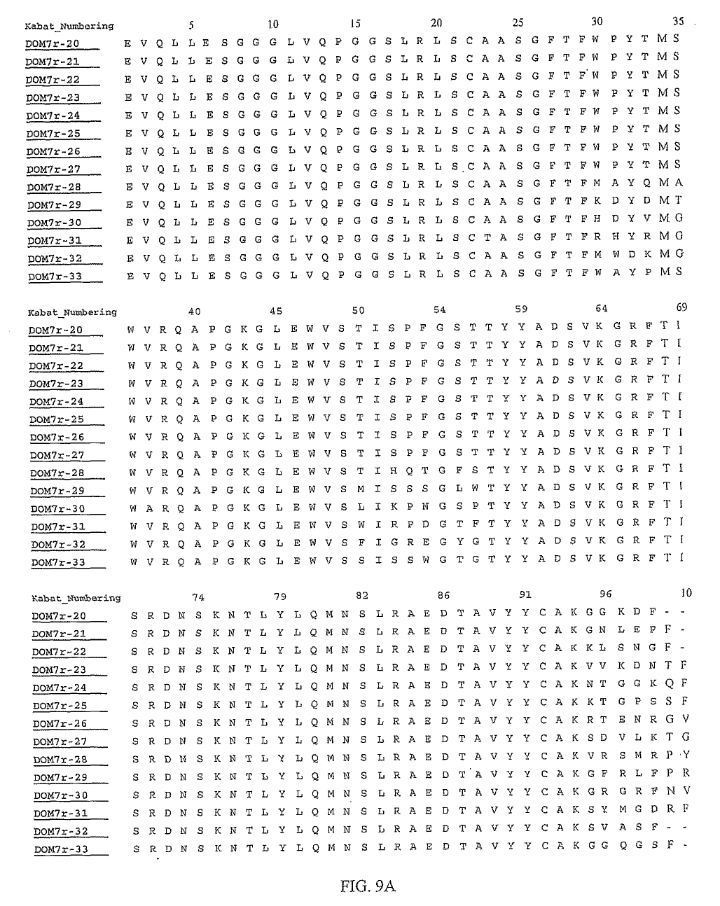

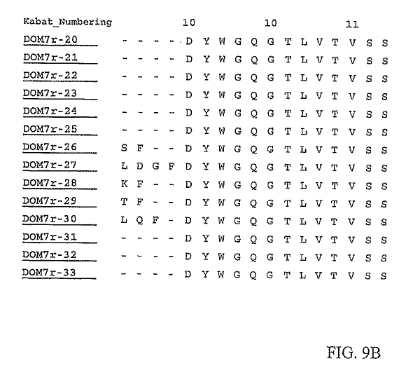

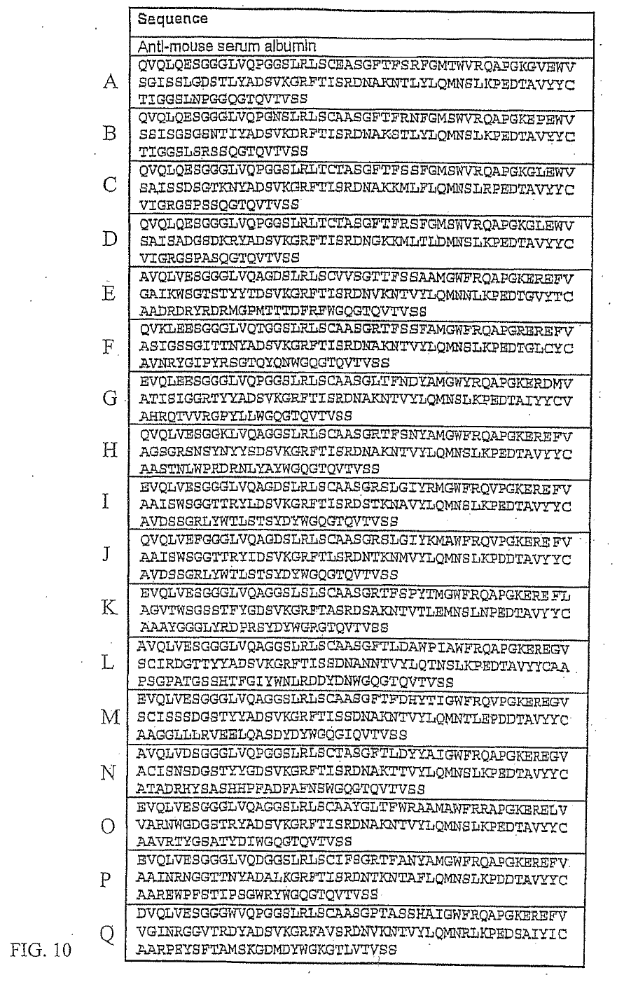

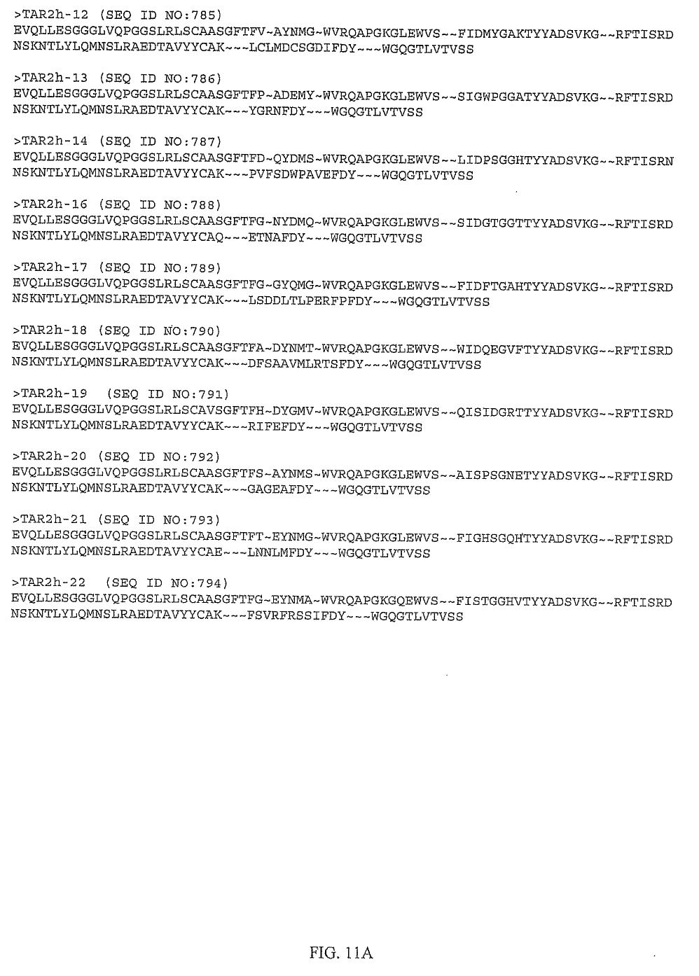

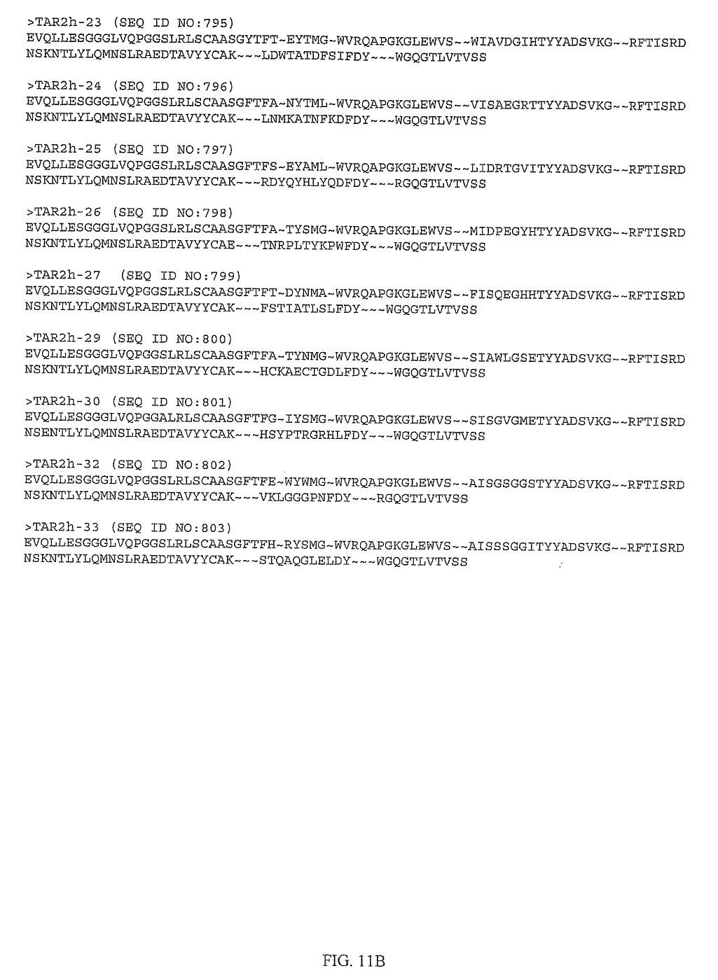

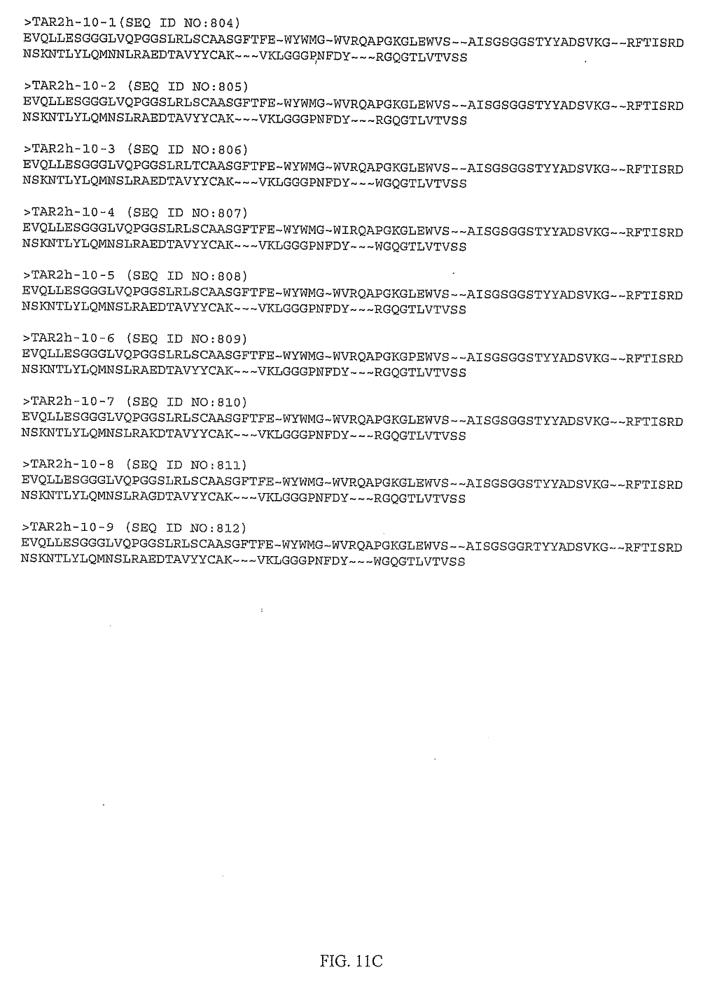

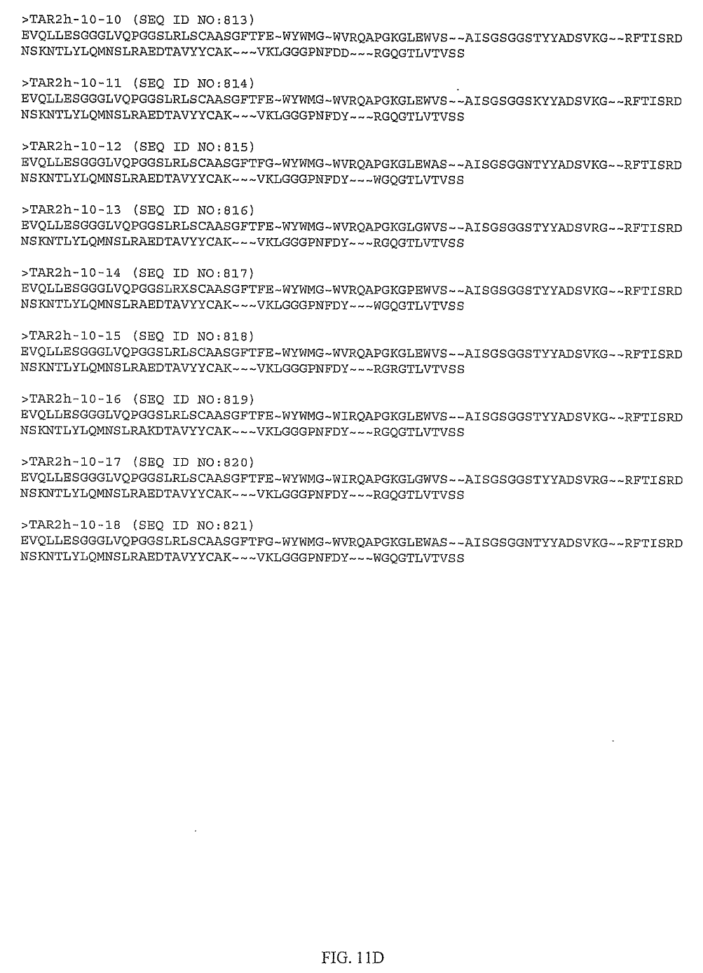

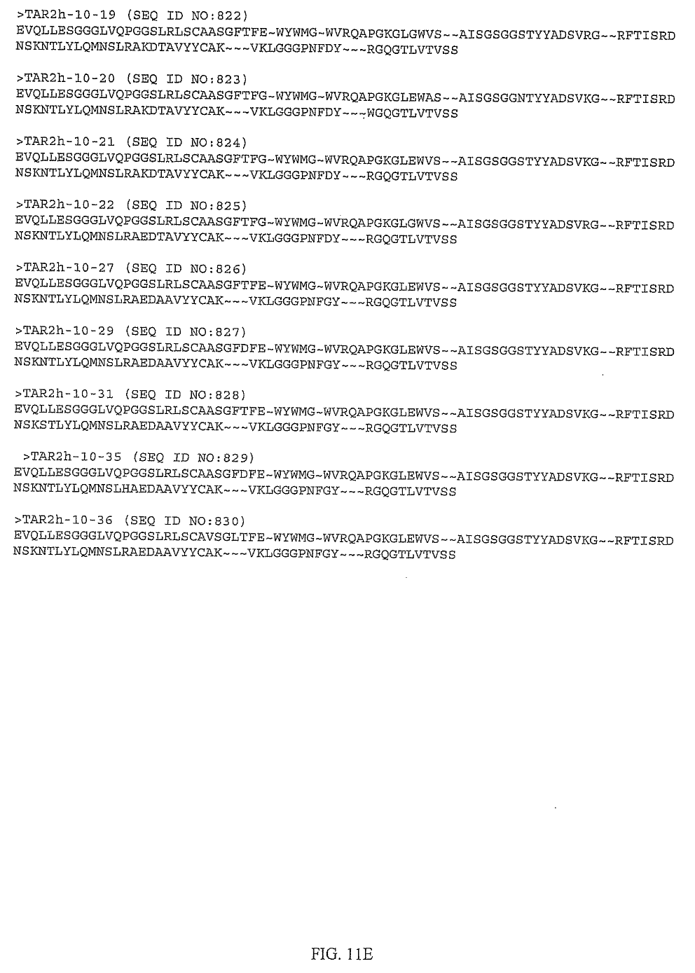

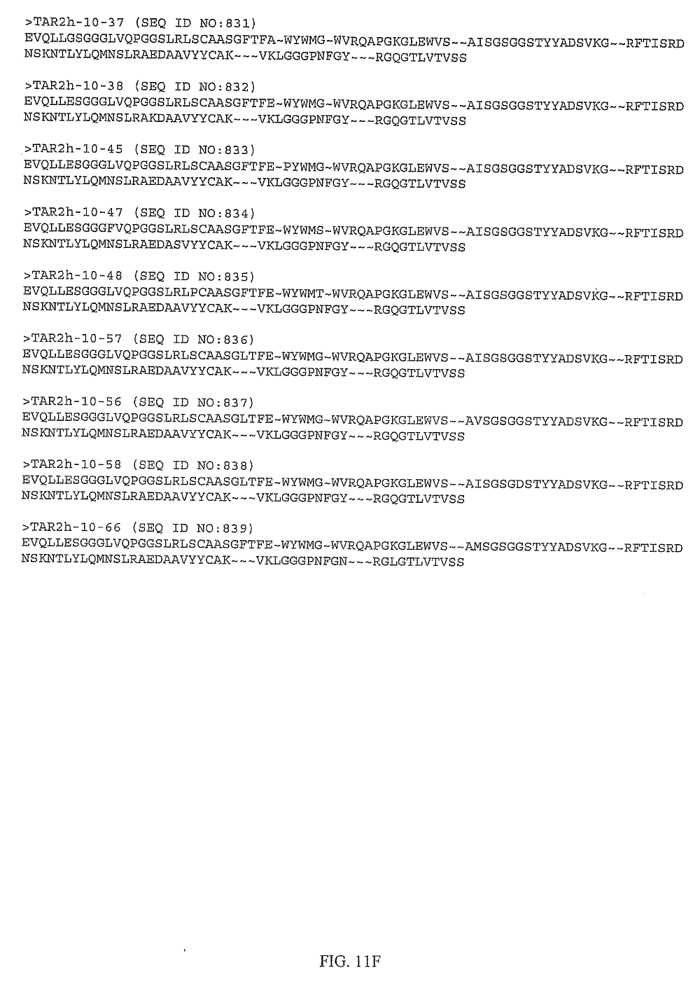

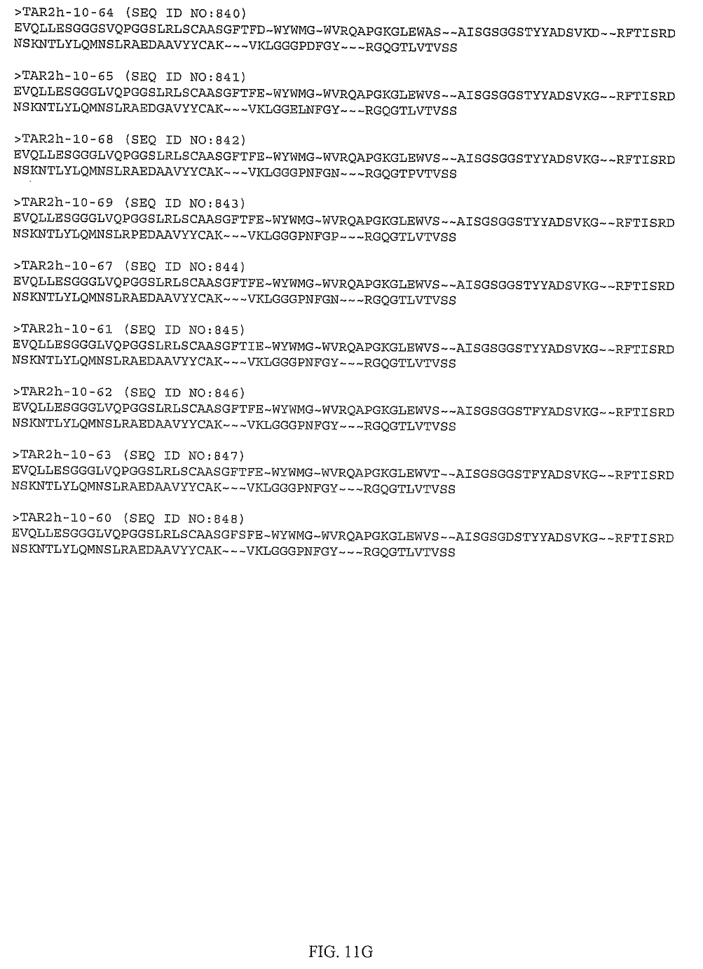

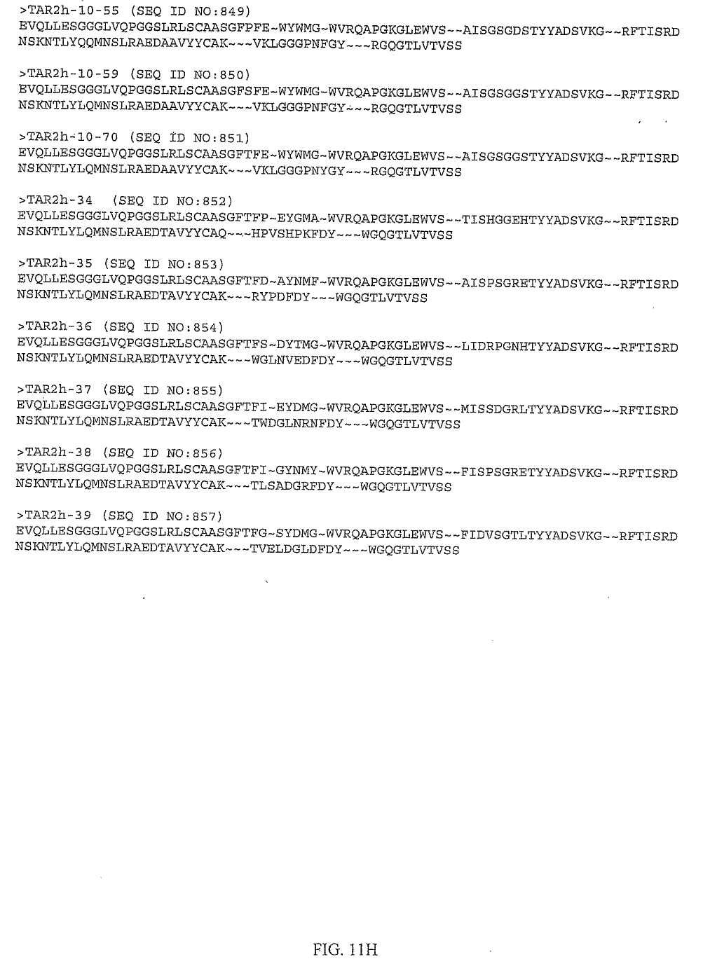

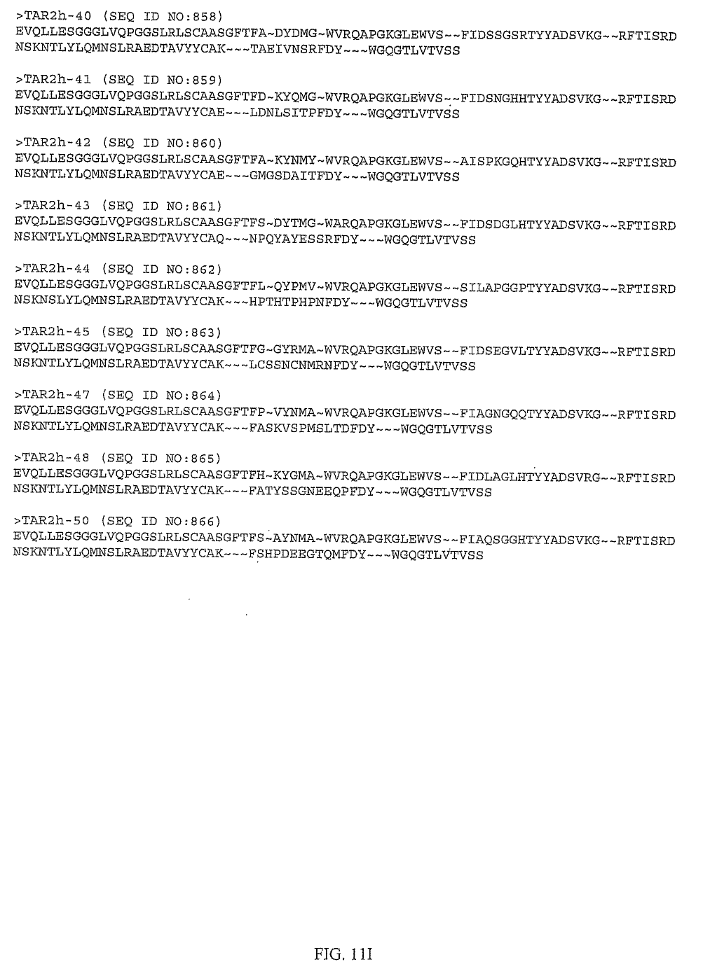

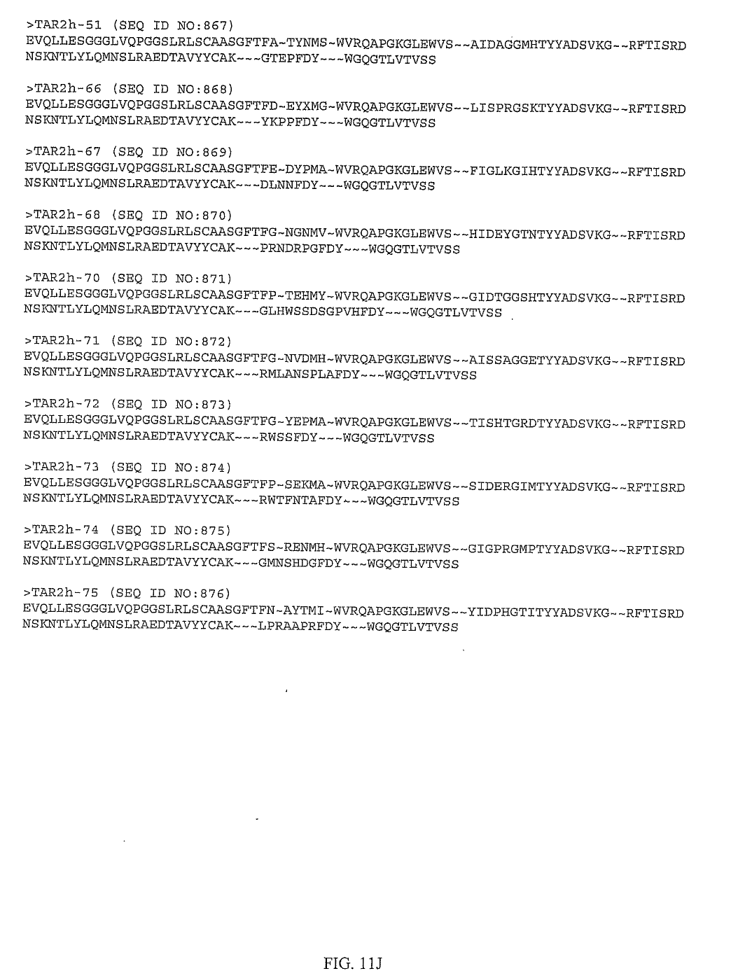

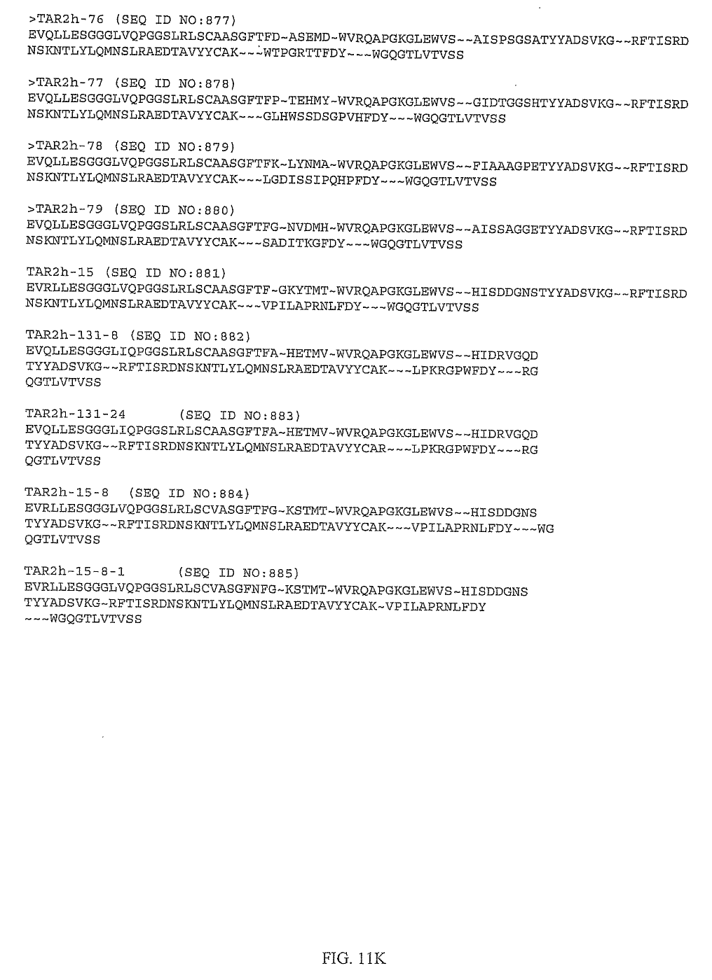

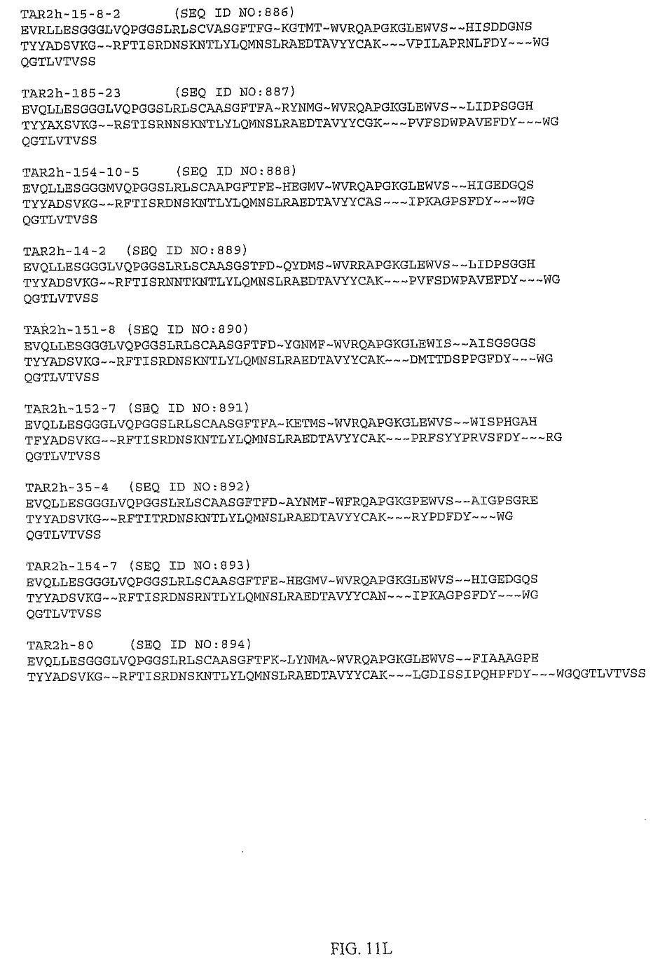

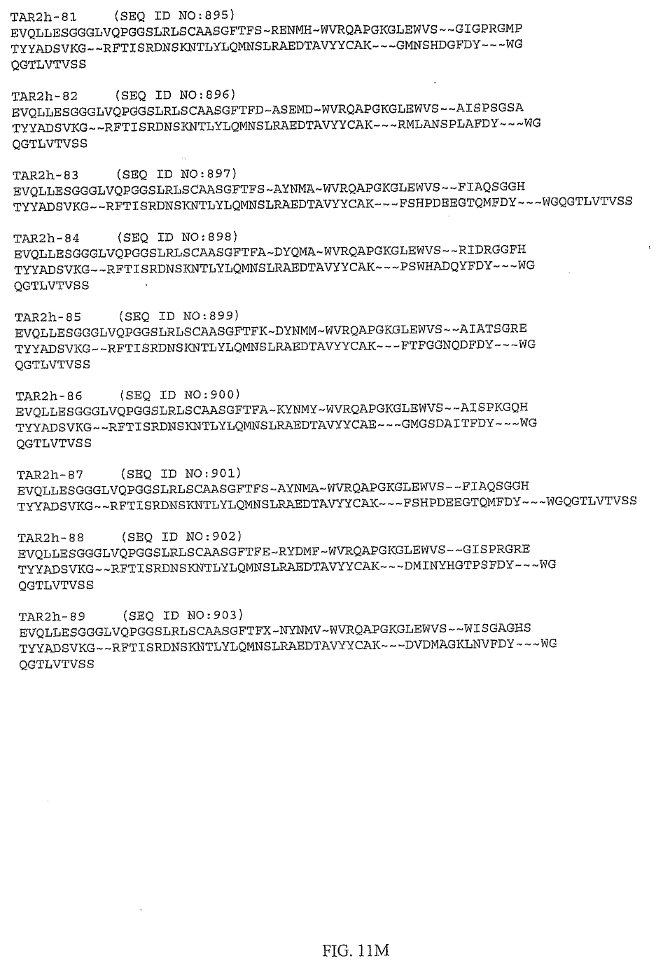

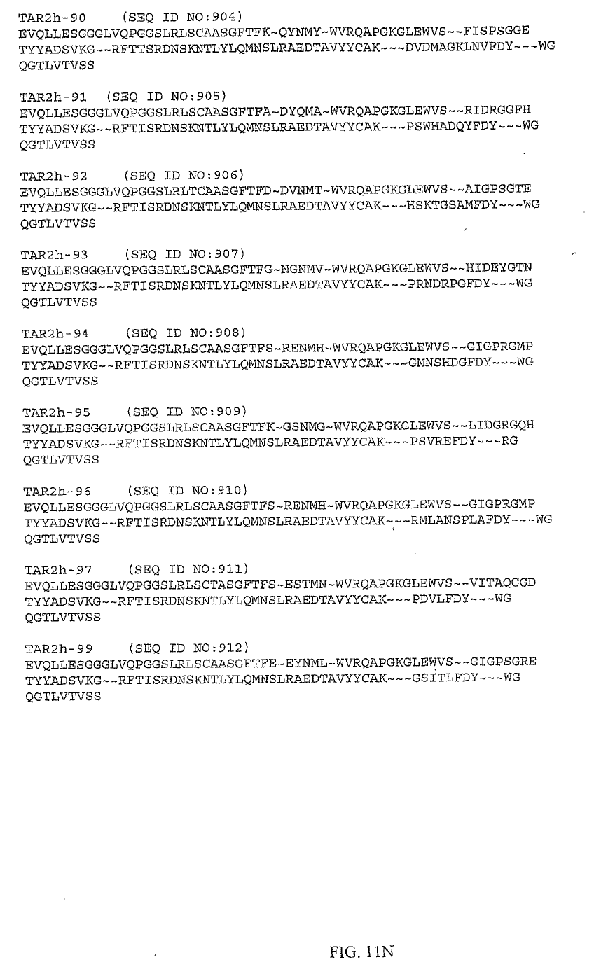

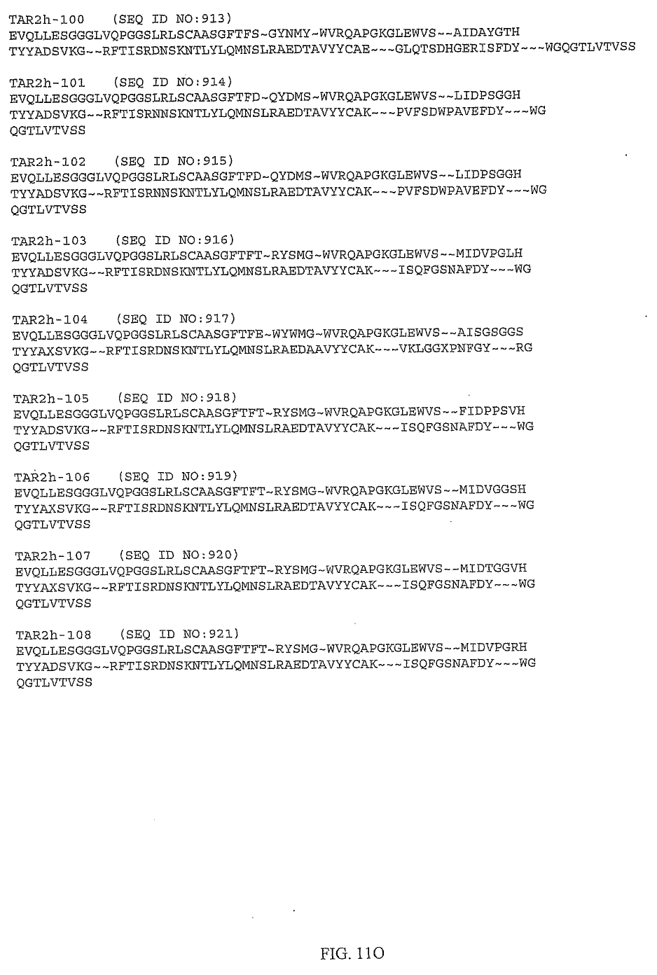

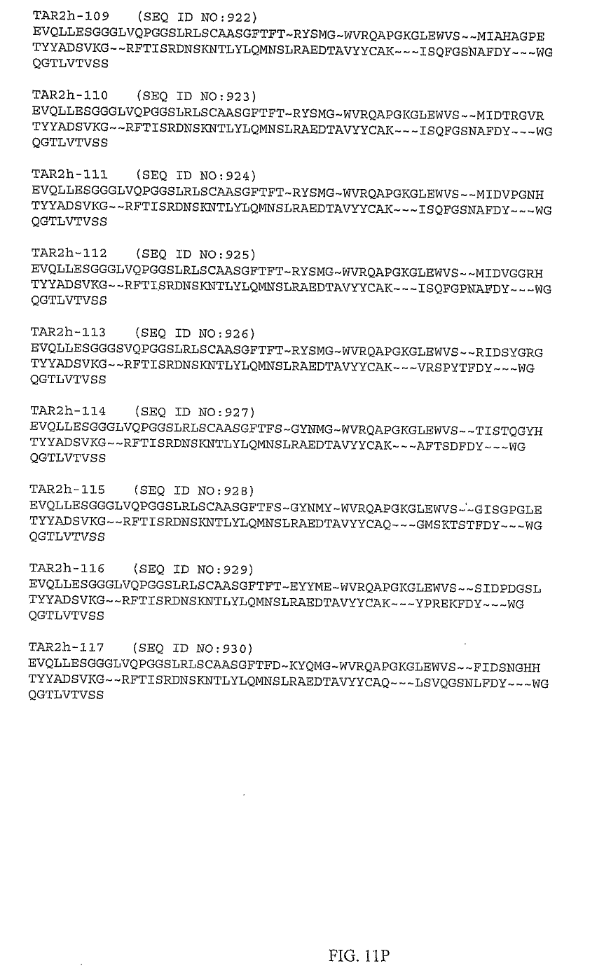

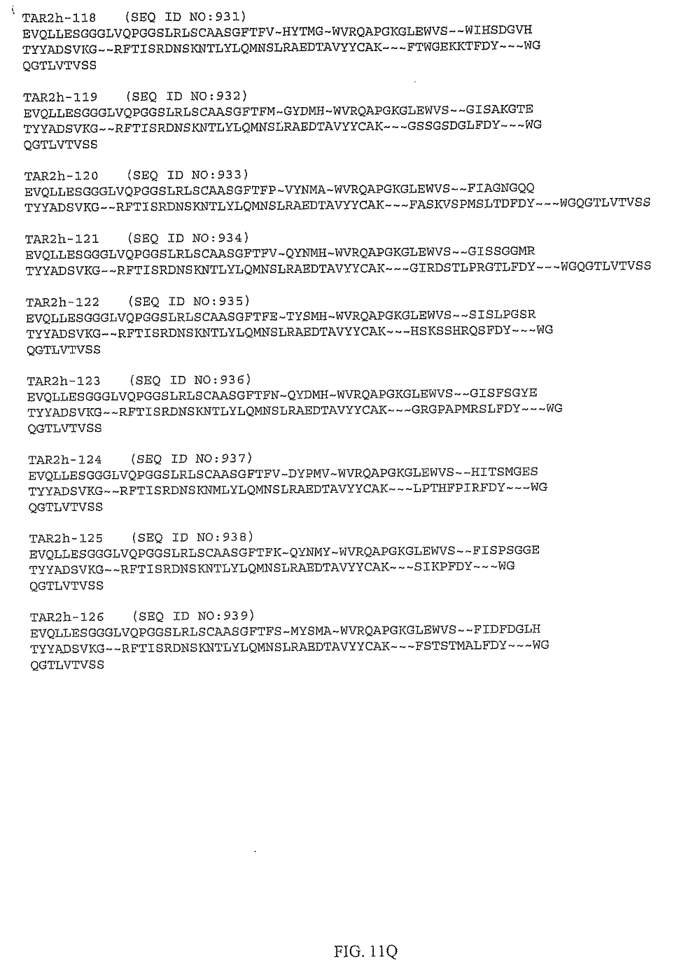

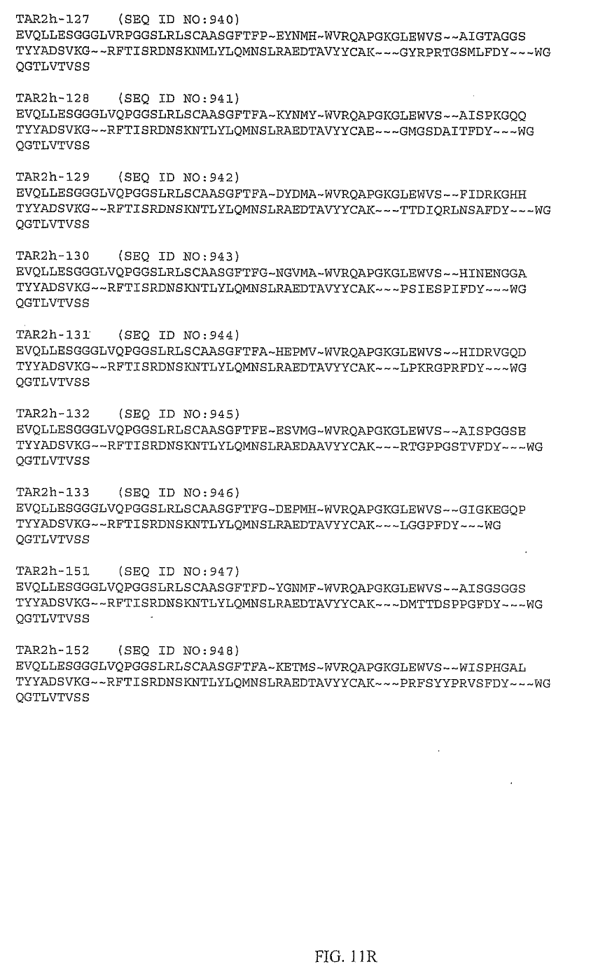

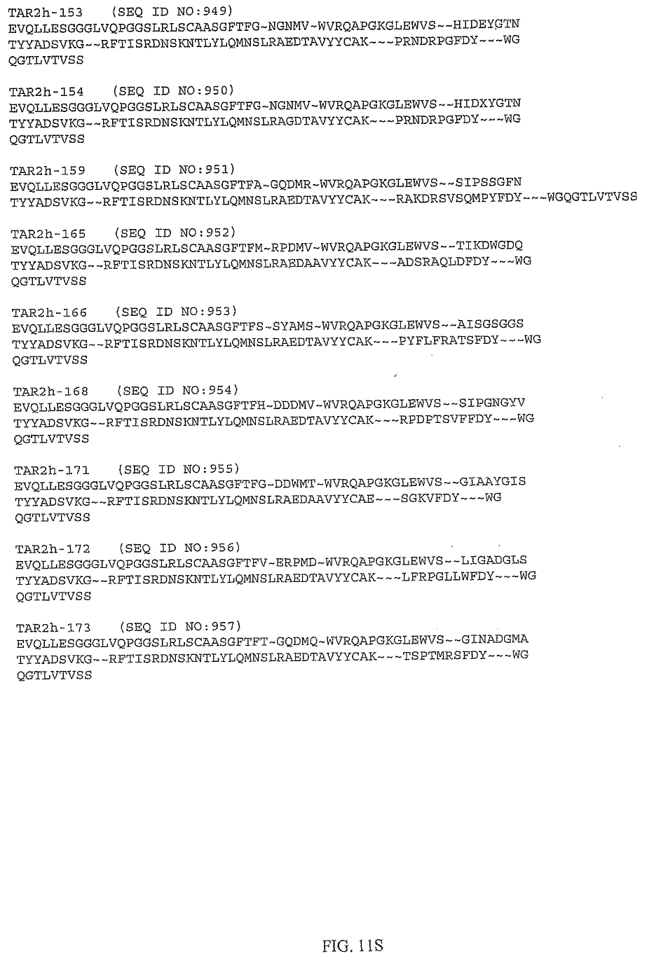

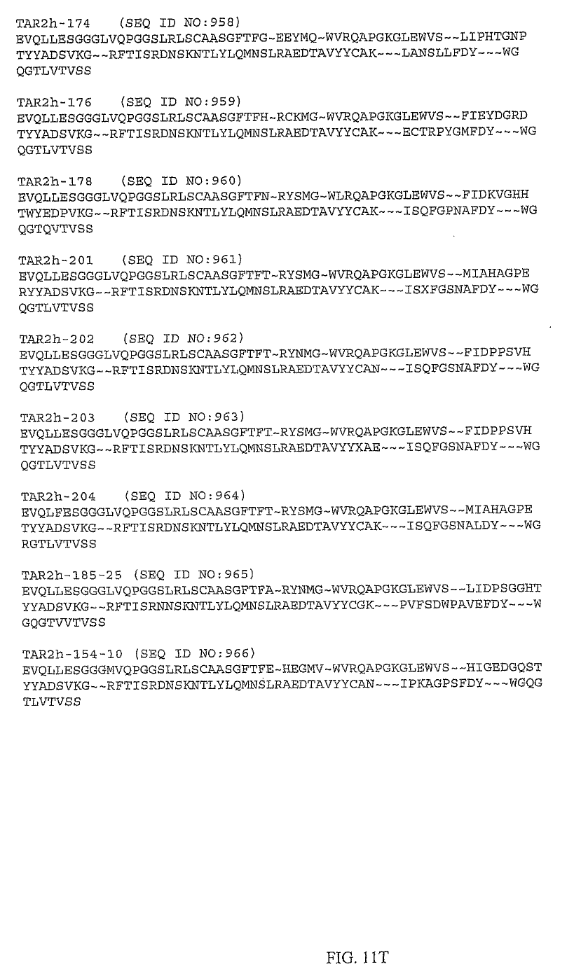

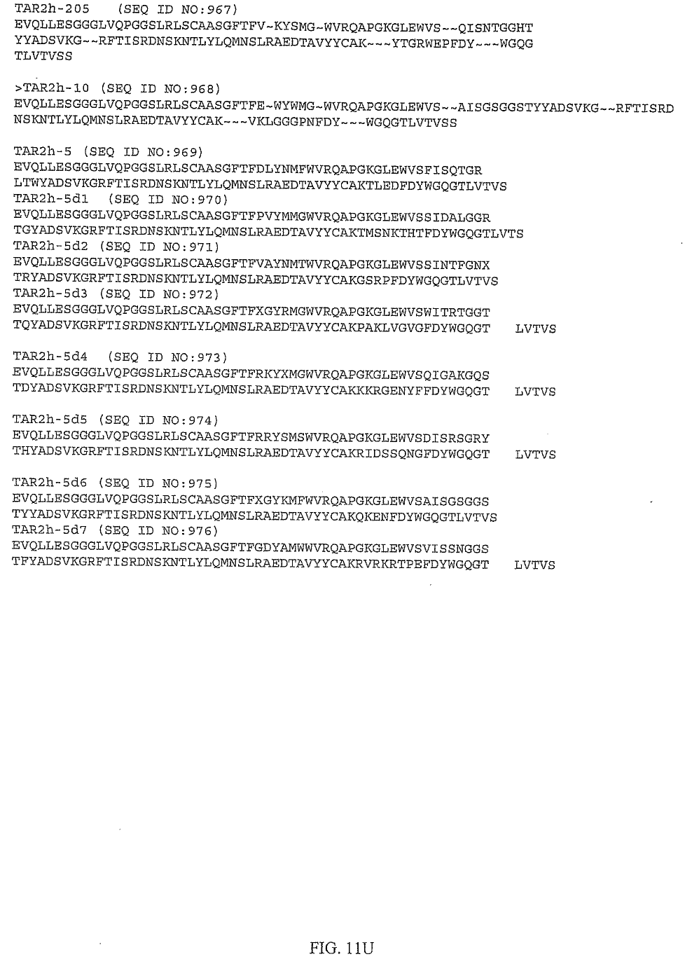

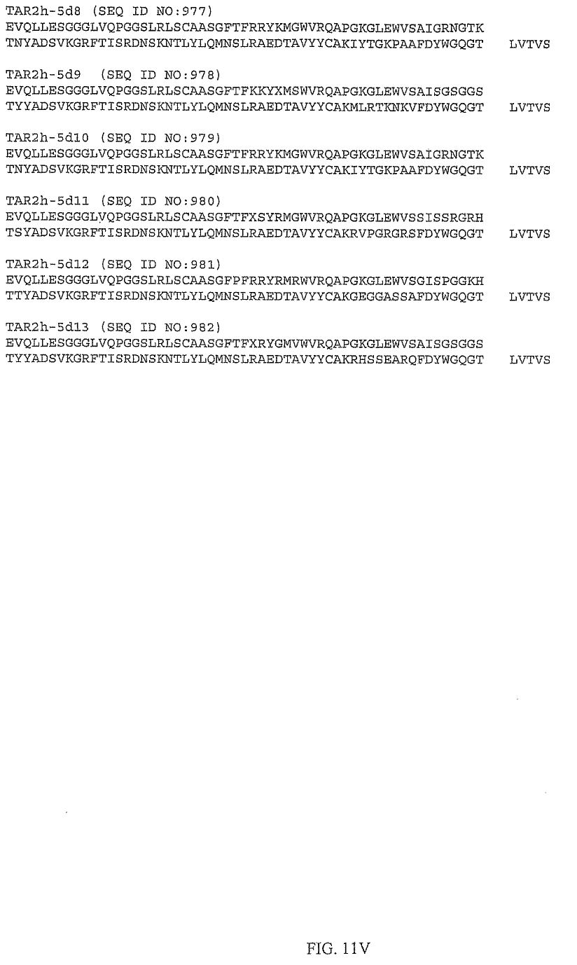

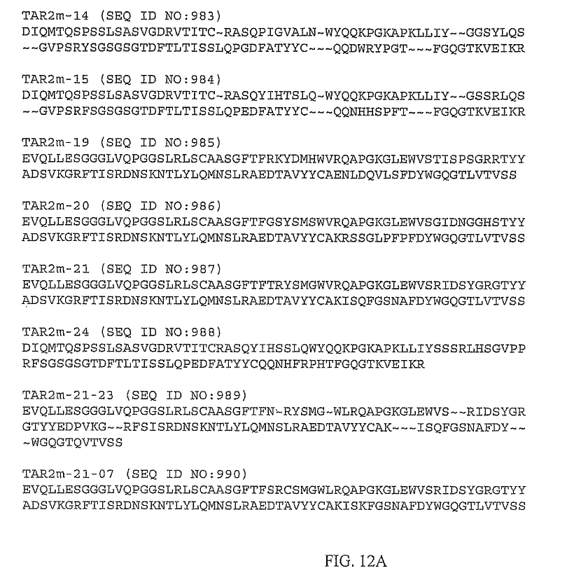

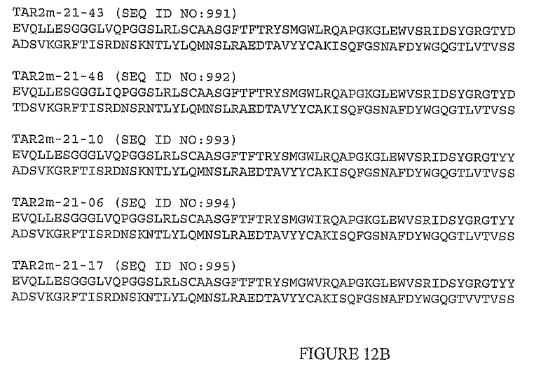

To determine the epitopic specificity of the anti-IL-1R1 dAbs, competitive binding assays were performed. In a study using the BIOCORE surface plasmon resonance instrument, IL-1ra was injected over a chip coupled with IL-1R1, and DOM4-122-23 or IL-1a was injected immediately after. The results are presented in FIGS. 3A and 3B. FIG. 3A shows that DOM4-122-23 bound to IL-1R1 that already had bound IL-1ra. When an injection of IL-1ra was followed by an injection of IL-1α, two molecules that are known to compete for binding to the receptor, the IL-1α was also unable to bind the receptor (FIG. 3B). The results were confirmed using a competition ELISA in which binding of IL-1ra to IL-1R1 in the presence of a DOM4-122-23 or IL-1a (in a series of concentrations) of was determined. The results of the ELISA showed that DOM4-122-23 did not inhibit binding of IL-1 ra (500 pM) to IL-1R1 even when present at concentrations up to 1 μM, wherein IL-1a did inhibit binding of IL-1ra to IL-1R1 (FIG. 4). The results confirmed that although DOM4-122-23 inhibits binding of IL-1 to IL-1R1, it does not compete with IL-1ra for binding to IL-1R1.

### Example 2

### Protease Stability

### Protease Stability

dAbs and ligands that comprise dAbs are useful for treating a variety of conditions, such as inflammatory conditions. In addition, as described herein, the half-life of dAbs and ligands can be tailored, for example, by PEGylation. Thus, dAbs and ligands can be administered, for example, systemically (e.g., PEGylated dAb to treat arthritis) or locally (e.g., dAb monomer to treat COPD).

The stability of two dAbs that bind IL-1R1 to the action of elastase or trypsin was investigated. Both of these proteases are found naturally at low levels within the lung, but in conditions such as COPD the levels of proteases, such as elastase, can become elevated. The dAb monomers DOM4-130-54, and a variant of DOM4-130-54 containing a point mutation that provides a cysteine residue for the specific attachment of PEG, were used in the study.

A 1 mg/ml solution of DOM4-130-54 in PBS was incubated with either 0.04% w/w trypsin or elastase (human sputum leucocyte elastase purchased from the Elastin Products Company Inc). The dAb/protease mixture was then incubated at 30° C. and samples were taken at defined time intervals (0, 1, 3 and 24 hrs) for SDS-PAGE analysis. At the given time points, the reaction was stopped by the addition SDS-PAGE loading buffer (×10 concentrated stock solution), followed by the snap freezing the samples in liquid nitrogen. Samples were analyzed by SDS-PAGE, and protein bands were visualized to reveal a time course for the protease degradation of the dAbs.

### Results

Two forms of DOM4-130-54 were tested for their stability to the action of elastase; E. coli expressed monomer and the cysteine engineered variant P80C expressed from P. pastoris. The P80C point mutation of DOM4-130-54 provides a cysteine residue for the specific attachment of PEG.

The time course for elastase degradation revealed that even after 24 hrs DOM4-130-54 showed no signs of degradation. The results also revealed that the introduction of the P80C mutation had no effect on the stability of the protein when compared to DOM4-130-54. These results indicate that the tertiary structure of the P80C variant does not substantially differ from the tertiary structure of DOM4-130-54.

The stability of the monomeric dAb DOM4-130-54 in the presence of trypsin was also tested. The time course for typsin degradation revealed that DOM4-130-54 was stable for at least 3 hours, and degradation was only seen at the 24 hr time point.

The results of this study revealed that dAbs are stable and resistant to elastase- or trypsin-mediated degradation. The demonstrated stability of dAbs to protease degradation indicates that dAbs can be administered in vivo and will remain functional for a sufficient amount of time to produce significant biological effects. For example, the results indicate that when dAbs are administered to the lung, they will be resistant to protease degradation and, thus, will be functional for a period of time that is sufficient to produce significant biological effects (e.g., bind and inhibit the activity of a target protein such as IL-1R1).

While this invention has been particularly shown and described with references to preferred embodiments thereof, it will be understood by those skilled in the art that various changes in form and details may be made therein without departing from the scope of the invention encompassed by the appended claims.

1. A domain antibody (dAb) monomer that has binding specificity for Interleukin-1 Receptor Type 1 (IL-1R1) and inhibits binding of Interleukin-1 (IL-1) to the receptor but does not inhibit binding of Interleukin-1 Receptor Antagonist (IL-1ra) to IL-1R1.

2. The dAb monomer of claim 1 wherein said IL-1 is selected from the group consisting of Interleukin-1α (IL-1α) and Interleukin-1β (IL-1β).

3. The dAb monomer of claim 1, wherein said dAb monomer inhibits binding of said IL-1 to IL-1R1 with an IC50 that is no greater than about 1 μM.

4. The dAb monomer of claim 1, wherein said dAb monomer inhibits IL-1-induced release of Interleukin-8 by MRC-5 cells (ATCC Accession No. CCL-171) in an in vitro assay with a ND50 that is ≦1 μM.

5. The dAb monomer of claim 4, wherein said dAb monomer inhibits IL-1-induced release of Interleukin-8 by MRC-5 cells (ATCC Accession No. CCL-171) in an in vitro assay with a ND50 that is ≦1 nM.

6. The dAb monomer of claim 1, wherein said dAb monomer inhibits IL-1-induced release of Interleukin-6 in a whole blood assay with a ND50 that is ≦1 μM.

7. The dAb monomer of claim 1, wherein one or more of the framework regions (FR) in said dAb monomer comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat.

8. The dAb monomer of claim 7, wherein the amino acid sequences of one or more framework regions in said dAb monomer are the same as the amino acid sequence of a corresponding framework region encoded by a human germline antibody gene segment, or the amino acid sequences of one or more of said framework regions collectively comprise up to 5 amino acid differences relative to the corresponding framework regions encoded by a human germline antibody gene segment.

9. The dAb monomer of claim 7, wherein the amino acid sequences of FR1, FR2, FR3 and FR4 in said dAb monomer are the same as the amino acid sequences of corresponding framework regions encoded by a human germline antibody gene segment, or the amino acid sequences of FR1, FR2, FR3 and FR4 collectively contain up to 10 amino acid differences relative to the corresponding framework regions encoded by a human germline antibody gene segment.

10. The dAb monomer of claim 7, wherein the dAb monomer comprises FR1, FR2 and FR3 regions, and the amino acid sequence of said FR1, FR2 and FR3 are the same as the amino acid sequences of corresponding framework regions encoded by a human germline antibody gene segment.

11. The dAb monomer of claim 7, wherein said human germline antibody gene segment is DPK9 and JK1

12. The dAb monomer of claim 1, wherein said dAb monomer competes for binding to IL-1R1 with a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173,) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-131 (SEQ ID NO:347), DOM4-132 (SEQ ID NO:348), and DOM4-133 (SEQ ID NO:349).

13. The dAb monomer of claim 12, wherein said dAb monomer comprises an amino acid sequence that has at least about 90% amino acid sequence identity with the amino acid sequence of a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173,) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-131 (SEQ ID NO:347), DOM4-132 (SEQ ID NO:348), and DOM4-133 (SEQ ID NO:349).

14. The dAb monomer of claim 1, wherein said dAb monomer binds human IL-1R1 with an affinity (KD) of about 300 nM to about 5 pM, as determined by surface plasmon resonance.

15. A ligand comprising a dAb monomer according to claim 1, and a half-life extending moiety.

16. The ligand of claim 15, wherein said half-life extending moiety is a polyalkylene glycol moiety, serum albumin or a fragment thereof, transferrin receptor or a transferrin-binding portion thereof, or an antibody or antibody fragment comprising a binding site for a polypeptide that enhances half-life in vivo.

17. The ligand of claim 16, wherein said half-life extending moiety is a polyethylene glycol moiety.

18. The ligand of claim 16, wherein said half-life extending moiety is an antibody or antibody fragment comprising a binding site for serum albumin or neonatal Fc receptor.

19. The ligand of claim 18, wherein said antibody or antibody fragment is an antibody fragment, and said antibody fragment is an immunoglobulin single variable domain.

20. The ligand of claim 19, wherein said immunoglobulin single variable domain competes for binding to human serum albumin with a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

21. The ligand of claim 20, wherein said immunoglobulin single variable domain binds human serum albumin comprises an amino acid sequence that has at least 90% amino acid sequence identity with the amino acid sequence of a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

22. A ligand comprising a dAb monomer that has binding specificity for IL-1R1 and inhibits binding of IL-1 to the receptor but does not inhibit binding of IL-1ra to IL-1R1, wherein said dAb monomer is selected from the group consisting of DOM4-122-23 (SEQ ID NO:1), and DOM4-122-24 (SEQ ID NO:2).

23. The ligand of claim 22, wherein said ligand is a dAb monomer.

24. The ligand of claim 22, wherein said ligand is a homodimer, homotrimer or homooligomer of said dAb monomer.

25. The ligand of claim 22, wherein said ligand is a heterodimer, heterotrimer or heterooligomer and comprises DOM4-122-23 (SEQ ID NO:1) and DOM4-122-24 (SEQ ID NO:2).

26. The ligand of claim 22, further comprising a dAb monomer that binds serum albumin.

27. The ligand of claim 26, wherein said dAb monomer that binds serum albumin is DOM7h-8 (SEQ ID NO:746).

28. The ligand of claim 27, wherein said ligand comprises DOM4-122-23 (SEQ ID NO:1) and DOM7h-8 (SEQ ID NO:746), or comprises DOM-122-24 (SEQ ID NO:2) and DOM7h-8 (SEQ ID NO:746).

29. A ligand comprising a dAb monomer that has binding specificity for IL-1R1 and inhibits binding of IL-1 to the receptor but does not inhibit binding of IL-1ra to IL-1R1, and a dAb monomer that has binding specificity for TNFR1.

30. The ligand of claim 29, wherein said dAb monomer that has binding specificity for IL-1R1 and inhibits binding of IL-1 and IL-1ra to IL-1R1 competes for binding to IL-1R1 with a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:01), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173,) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-131 (SEQ ID NO:347), DOM4-132 (SEQ ID NO:348), and DOM4-133 (SEQ ID NO:349).

31. The ligand of claim 30, wherein said dAb monomer that has binding specificity for IL-1R1 and inhibits binding of IL-1 and IL-1ra to IL-1R1 comprises an amino acid sequence that has at least about 90% amino acid sequence identity with the amino acid sequence of a dAb selected from the group consisting of DOM4-122-23 (SEQ ID NO:1), DOM4-122-24 (SEQ ID NO:2), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173,) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-131 (SEQ ID NO:347), DOM4-132 (SEQ ID NO:348), and DOM4-133 (SEQ ID NO:349).

32. The ligand of claim 29, wherein said dAb monomer that has binding specificity for TNFR1 competes for binding to TNFR1 with a dAb selected from the group consisting of TAR2h-12 (SEQ ID NO:785), TAR2h-13 (SEQ ID NO:786), TAR2h-14 (SEQ ID NO:787), TAR2h-16 (SEQ ID NO:788), TAR2h-17 (SEQ ID NO:789), TAR2h-18 (SEQ ID NO:790), TAR2h-19 (SEQ ID NO:791), TAR2h-20 (SEQ ID NO:792), TAR2h-21 (SEQ ID NO:793), TAR2h-22 (SEQ ID NO:794), TAR2h-23 (SEQ ID NO:795), TAR2h-24 (SEQ ID NO:796), TAR2h-25 (SEQ ID NO:797), TAR2h-26 (SEQ ID NO:798), TAR2h-27 (SEQ ID NO:799), TAR2h-29 (SEQ ID NO:800), TAR2h-30 (SEQ ID NO:801), TAR2h-32 (SEQ ID NO:802), TAR2h-33 (SEQ ID NO:803), TAR2h-10⁻¹ (SEQ ID NO:804), TAR2h-10-2 (SEQ ID NO:805), TAR2h-10-3 (SEQ ID NO:806), TAR2h-10⁻⁴ (SEQ ID NO:807), TAR2h-10-5 (SEQ ID NO:808), TAR2h-10-6 (SEQ ID NO:809), TAR2h-10-7 (SEQ ID NO:810), TAR2h-10-8 (SEQ ID NO:811), TAR2h-10⁻⁹ (SEQ ID NO:812), TAR2h-10-10 (SEQ ID NO:813), TAR2h-10-11 (SEQ ID NO:814), TAR2h-10-12 (SEQ ID NO:815), TAR2h-10-13 (SEQ ID NO:816), TAR2h-10-14 (SEQ ID NO:817), TAR2h-10-15 (SEQ ID NO:818), TAR2h-10-16 (SEQ ID NO:819), TAR2h-10-17 (SEQ ID NO:820), TAR2h-10-18 (SEQ ID NO:821), TAR2h-10-19 (SEQ ID NO:822), TAR2h-10-20 (SEQ ID NO:823), TAR2h-10-21 (SEQ ID NO:824), TAR2h-10-22 (SEQ ID NO:825), TAR2h-10-27 (SEQ ID NO:826), TAR2h-10-29 (SEQ ID NO:827), TAR2h-10-31 (SEQ ID NO:828), TAR2h-10-35 (SEQ ID NO:829), TAR2h-10-36 (SEQ ID NO:830), TAR2h-10-37 (SEQ ID NO:831), TAR2h-10-38 (SEQ ID NO:832), TAR2h-10-45 (SEQ ID NO:833), TAR2h-10-47 (SEQ ID NO:834), TAR2h-10-48 (SEQ ID NO:835), TAR2h-10-57 (SEQ ID NO:836), TAR2h-10-56 SEQ ID NO:837), TAR2h-10-58 (SEQ ID NO:838), TAR2h-10-66 (SEQ ID NO:839), TAR2h-10-64 (SEQ ID NO:840), TAR2h-10-65 (SEQ ID NO:841), TAR2h-10-68 (SEQ ID NO:842), TAR2h-10-69 (SEQ ID NO:843), TAR2h-10-67 (SEQ ID NO:844), TAR2h-10-61 (SEQ ID NO:845), TAR2h-10-62 (SEQ ID NO:846), TAR2h-10-63 (SEQ ID NO:847), TAR2h-10-60 (SEQ ID NO:848), TAR2h-10-55 (SEQ ID NO:849), TAR2h-10-59 (SEQ ID NO:850), TAR2h-10-70 (SEQ ID NO:851), TAR2h-34 (SEQ ID NO:852), TAR2h-35 (SEQ ID NO:853), TAR2h-36 (SEQ ID NO:854), TAR2h-37 (SEQ ID NO:855), TAR2h-38 (SEQ ID NO:856), TAR2h-39 (SEQ ID NO:857), TAR2h-40 (SEQ ID NO:858), TAR2h-41 (SEQ ID NO:859), TAR2h-42 (SEQ ID NO:860), TAR2h-43 (SEQ ID NO:861), TAR2h-44 (SEQ ID NO:862), TAR2h-45 (SEQ ID NO:863), TAR2h-47 (SEQ ID NO:864), TAR2h-48 (SEQ ID NO:865), TAR2h-50 (SEQ ID NO:866), TAR2h-51 (SEQ ID NO:867), TAR2h-66 (SEQ ID NO:868), TAR2h-67 (SEQ ID NO:869), TAR2h-68 (SEQ ID NO:870), TAR2h-70 (SEQ ID NO:871), TAR2h-71 (SEQ ID NO:872), TAR2h-72 (SEQ ID NO:873), TAR2h-73 (SEQ ID NO:874), TAR2h-74 (SEQ ID NO:875), TAR2h-75 (SEQ ID NO:876), TAR2h-76 (SEQ ID NO:877), TAR2h-77 (SEQ ID NO:878), TAR2h-78 (SEQ ID NO:879), TAR2h-79 (SEQ ID NO:880), TAR2h-15 (SEQ ID NO:881), TAR2h-131-8 (SEQ ID NO:882), TAR2h-131-24 (SEQ ID NO:883), TAR2h-15-8 (SEQ ID NO:884), TAR2h-15-8-1 (SEQ ID NO:885), TAR2h-15-8-2 (SEQ ID NO:886), TAR2h-185-23 (SEQ ID NO:887), TAR2h-154-10⁻⁵ (SEQ ID NO:888), TAR2h-14-2 (SEQ ID NO:889), TAR2h-151-8 (SEQ ID NO:890), TAR2h-152-7 (SEQ ID NO:891), TAR2h-35-4 (SEQ ID NO:892), TAR2h-154-7 (SEQ ID NO:893), TAR2h-80 (SEQ ID NO:894), TAR2h-81 (SEQ ID NO:895), TAR2h-82 (SEQ ID NO:896), TAR2h-83 (SEQ ID NO:897), TAR2h-84 (SEQ ID NO:898), TAR2h-85 (SEQ ID NO:899), TAR2h-86 (SEQ ID NO:900), TAR2h-87 (SEQ ID NO:901), TAR2h-88 (SEQ ID NO:902), TAR2h-89 (SEQ ID NO:903), TAR2h-90 (SEQ ID NO:904), TAR2h-91 (SEQ ID NO:905), TAR2h-92 (SEQ ID NO:906), TAR2h-93 (SEQ ID NO:907), TAR2h-94 (SEQ ID NO:908), TAR2h-95 (SEQ ID NO:909), TAR2h-96 (SEQ ID NO:910), TAR2h-97 (SEQ ID NO:911), TAR2h-99 (SEQ ID NO:912), TAR2h-100(SEQ ID NO:913), TAR2h-101 (SEQ ID NO:914), TAR2h-102 (SEQ ID NO:915), TAR2h-103 (SEQ ID NO:916), TAR2h-104 (SEQ ID NO:917), TAR2h-105 (SEQ ID NO:918), TAR2h-106 (SEQ ID NO:919), TAR2h-107 (SEQ ID NO:920), TAR2h-108 (SEQ ID NO:921), TAR2h-109 (SEQ ID NO:922), TAR2h-110 (SEQ ID NO:923), TAR2h-111 (SEQ ID NO:924), TAR2h-112 (SEQ ID NO:925), TAR2h-113 (SEQ ID NO:926), TAR2h-114 (SEQ ID NO:927), TAR2h-115 (SEQ ID NO:928), TAR2h-116 (SEQ ID NO:929), TAR2h-117 (SEQ ID NO:930), TAR2h-118 (SEQ ID NO:931), TAR2h-119 (SEQ ID NO:932), TAR2h-120 (SEQ ID NO:933), TAR2h-121 (SEQ ID NO:934), TAR2h-122 (SEQ ID NO:935), TAR2h-123 (SEQ ID NO:936), TAR2h-124 (SEQ ID NO:937), TAR2h-125 (SEQ ID NO:938), TAR2h-126 (SEQ ID NO:939), TAR2h-127 (SEQ ID NO:940), TAR2h-128 (SEQ ID NO:941), TAR2h-129 (SEQ ID NO:942), TAR2h-130 (SEQ ID NO:943), TAR2h-131 (SEQ ID NO:944), TAR2h-132 (SEQ ID NO:945), TAR2h-133 (SEQ ID NO:946), TAR2h-151 (SEQ ID NO:947), TAR2h-152 (SEQ ID NO:948), TAR2h-153 (SEQ ID NO:949), TAR2h-154 (SEQ ID NO:950), TAR2h-159 (SEQ ID NO:951), TAR2h-165 (SEQ ID NO:952), TAR2h-166 (SEQ ID NO:953), TAR2h-168 (SEQ ID NO:954), TAR2h-171 (SEQ ID NO:955), TAR2h-172 (SEQ ID NO:956), TAR2h-173 (SEQ ID NO:957), TAR2h-174 (SEQ ID NO:958), TAR2h-176 (SEQ ID NO:959), TAR2h-178 (SEQ ID NO:960), TAR2h-201 (SEQ ID NO:961), TAR2h-202 (SEQ ID NO:962), TAR2h-203 (SEQ ID NO:963), TAR2h-204 (SEQ ID NO:964), TAR2h-185-25 (SEQ ID NO:965), TAR2h-154-10 SEQ ID NO:966), TAR2h-205 (SEQ ID NO:967), TAR2h-10 (SEQ ID NO:968), TAR2h-5 (SEQ ID NO:969), TAR2h-5d1 (SEQ ID NO:970), TAR2h-5d2 (SEQ ID NO:971), TAR2h-5d3 (SEQ ID NO:972), TAR2h-5d4 (SEQ ID NO:973), TAR2h-5d5 (SEQ ID NO:974), TAR2h-5d6 (SEQ ID NO:975), TAR2h-5d7 (SEQ ID NO:976), TAR2h-5d8 (SEQ ID NO:977), TAR2h-5d9 (SEQ ID NO:978), TAR2h-5d10 (SEQ ID NO:979), TAR2h-5d 11 (SEQ ID NO:980), TAR2h-5d12 (SEQ ID NO:981), and TAR2h-5d13 (SEQ ID NO:982).

33. The ligand of claim 32, wherein said dAb monomer that has binding specificity for TNFR1 comprises an amino acid sequence that has at least about 90% amino acid sequence identity with the amino acid sequence of a dAb selected from the group consisting of TAR2h-12 (SEQ ID NO:785), TAR2h-13 (SEQ ID NO:786), TAR2h-14 (SEQ ID NO:787), TAR2h-16 (SEQ ID NO:788), TAR2h-17 (SEQ ID NO:789), TAR2h-18 (SEQ ID NO:790), TAR2h-19 (SEQ ID NO:791), TAR2h-20 (SEQ ID NO:792), TAR2h-21 (SEQ ID NO:793), TAR2h-22 (SEQ ID NO:794), TAR2h-23 (SEQ ID NO:795), TAR2h-24 (SEQ ID NO:796), TAR2h-25 (SEQ ID NO:797), TAR2h-26 (SEQ ID NO:798), TAR2h-27 (SEQ ID NO:799), TAR2h-29 (SEQ ID NO:800), TAR2h-30 (SEQ ID NO:801), TAR2h-32 (SEQ ID NO:802), TAR2h-33 (SEQ ID NO:803), TAR2h-10-1 (SEQ ID NO:804), TAR2h-10-2 (SEQ ID NO:805), TAR2h-10⁻³ (SEQ ID NO:806), TAR2h-10⁻⁴ (SEQ ID NO:807), TAR2h-10⁻⁵ (SEQ ID NO:808), TAR2h-10-6 (SEQ ID NO:809), TAR2h-10⁻⁷ (SEQ ID NO:810), TAR2h-10⁻⁸ (SEQ ID NO:811), TAR2h-10-9 (SEQ ID NO:812), TAR2h-10-10 (SEQ ID NO:813), TAR2h-10-11 (SEQ ID NO:814), TAR2h-10-12 (SEQ ID NO:815), TAR2h-10-13 (SEQ ID NO:816), TAR2h-10-14 (SEQ ID NO:817), TAR2h-10-15 (SEQ ID NO:818), TAR2h-10-16 (SEQ ID NO:819), TAR2h-10-17 (SEQ ID NO:820), TAR2h-10-18 (SEQ ID NO:821), TAR2h-10-19 (SEQ ID NO:822), TAR2h-10-20 (SEQ ID NO:823), TAR2h-10-21 (SEQ ID NO:824), TAR2h-10-22 (SEQ ID NO:825), TAR2h-10-27 (SEQ ID NO:826), TAR2h-10-29 (SEQ ID NO:827), TAR2h-10-31 (SEQ ID NO:828), TAR2h-10-35 (SEQ ID NO:829), TAR2h-10-36 (SEQ ID NO:830), TAR2h-10-37 (SEQ ID NO:831), TAR2h-10-38 (SEQ ID NO:832), TAR2h-10-45 (SEQ ID NO:833), TAR2h-10-47 (SEQ ID NO:834), TAR2h-10-48 (SEQ ID NO:835), TAR2h-10-57 (SEQ ID NO:836), TAR2h-10-56 SEQ ID NO:837), TAR2h-10-58 (SEQ ID NO:838), TAR2h-10-66 (SEQ ID NO:839), TAR2h-10-64 (SEQ ID NO:840), TAR2h-10-65 (SEQ ID NO:841), TAR2h-10-68 (SEQ ID NO:842), TAR2h-10-69 (SEQ ID NO:843), TAR2h-10-67 (SEQ ID NO:844), TAR2h-10-61 (SEQ ID NO:845), TAR2h-10-62 (SEQ ID NO:846), TAR2h-10-63 (SEQ ID NO:847), TAR2h-10-60 (SEQ ID NO:848), TAR2h-10-55 (SEQ ID NO:849), TAR2h-10-59 (SEQ ID NO:850), TAR2h-10-70 (SEQ ID NO:851), TAR2h-34 (SEQ ID NO:852), TAR2h-35 (SEQ ID NO:853), TAR2h-36 (SEQ ID NO:854), TAR2h-37 (SEQ ID NO:855), TAR2h-38 (SEQ ID NO:856), TAR2h-39 (SEQ ID NO:857), TAR2h-40 (SEQ ID NO:858), TAR2h-41 (SEQ ID NO:859), TAR2h-42 (SEQ ID NO:860), TAR2h-43 (SEQ ID NO:861), TAR2h-44 (SEQ ID NO:862), TAR2h-45 (SEQ ID NO:863), TAR2h-47 (SEQ ID NO:864), TAR2h-48 (SEQ ID NO:865), TAR2h-50 (SEQ ID NO:866), TAR2h-51 (SEQ ID NO:867), TAR2h-66 (SEQ ID NO:868), TAR2h-67 (SEQ ID NO:869), TAR2h-68 (SEQ ID NO:870), TAR2h-70 (SEQ ID NO:871), TAR2h-71 (SEQ ID NO:872), TAR2h-72 (SEQ ID NO:873), TAR2h-73 (SEQ ID NO:874), TAR2h-74 (SEQ ID NO:875), TAR2h-75 (SEQ ID NO:876), TAR2h-76 (SEQ ID NO:877), TAR2h-77 (SEQ ID NO:878), TAR2h-78 (SEQ ID NO:879), TAR2h-79 (SEQ ID NO:880), TAR2h-15 (SEQ ID NO:881), TAR2h-131-8 (SEQ ID NO:882), TAR2h-131-24 (SEQ ID NO:883), TAR2h-15-8 (SEQ ID NO:884), TAR2h-15-8-1 (SEQ ID NO:885), TAR2h-15-8-2 (SEQ ID NO:886), TAR2h-185-23 (SEQ ID NO:887), TAR2h-154-10-5 (SEQ ID NO:888), TAR2h-14-2 (SEQ ID NO:889), TAR2h-151-8 (SEQ ID NO:890), TAR2h-152-7 (SEQ ID NO:891), TAR2h-35-4 (SEQ ID NO:892), TAR2h-154-7 (SEQ ID NO:893), TAR2h-80 (SEQ ID NO:894), TAR2h-81 (SEQ ID NO:895), TAR2h-82 (SEQ ID NO:896), TAR2h-83 (SEQ ID NO:897), TAR2h-84 (SEQ ID NO:898), TAR2h-85 (SEQ ID NO:899), TAR2h-86 (SEQ ID NO:900), TAR2h-87 (SEQ ID NO:901), TAR2h-88 (SEQ ID NO:902), TAR2h-89 (SEQ ID NO:903), TAR2h-90 (SEQ ID NO:904), TAR2h-91 (SEQ ID NO:905), TAR2h-92 (SEQ ID NO:906), TAR2h-93 (SEQ ID NO:907), TAR2h-94 (SEQ ID NO:908), TAR2h-95 (SEQ ID NO:909), TAR2h-96 (SEQ ID NO:910), TAR2h-97 (SEQ ID NO:911), TAR2h-99 (SEQ ID NO:912), TAR2h-100(SEQ ID NO:913), TAR2h-101 (SEQ ID NO:914), TAR2h-102 (SEQ ID NO:915), TAR2h-103 (SEQ ID NO:916), TAR2h-104 (SEQ ID NO:917), TAR2h-105 (SEQ ID NO:918), TAR2h-106 (SEQ ID NO:919), TAR2h-107 (SEQ ID NO:920), TAR2h-108 (SEQ ID NO:921), TAR2h-09 (SEQ ID NO:922), TAR2h-110 (SEQ ID NO:923), TAR2h-111 (SEQ ID NO:924), TAR2h-112 (SEQ ID NO:925), TAR2h-113 (SEQ ID NO:926), TAR2h-114 (SEQ ID NO:927), TAR2h-115 (SEQ ID NO:928), TAR2h-116 (SEQ ID NO:929), TAR2h-117 (SEQ ID NO:930), TAR2h-118 (SEQ ID NO:931), TAR2h-119 (SEQ ID NO:932), TAR2h-120 (SEQ ID NO:933), TAR2h-121 (SEQ ID NO:934), TAR2h-122 (SEQ ID NO:935), TAR2h-123 (SEQ ID NO:936), TAR2h-124 (SEQ ID NO:937), TAR2h-125 (SEQ ID NO:938), TAR2h-126 (SEQ ID NO:939), TAR2h-127 (SEQ ID NO:940), TAR2h-128 (SEQ ID NO:941), TAR2h-129 (SEQ ID NO:942), TAR2h-130 (SEQ ID NO:943), TAR2h-131 (SEQ ID NO:944), TAR2h-132 (SEQ ID NO:945), TAR2h-133 (SEQ ID NO:946), TAR2h-151 (SEQ ID NO:947), TAR2h-152 (SEQ ID NO:948), TAR2h-153 (SEQ ID NO:949), TAR2h-154 (SEQ ID NO:950), TAR2h-159 (SEQ ID NO:951), TAR2h-165 (SEQ ID NO:952), TAR2h-166 (SEQ ID NO:953), TAR2h-168 (SEQ ID NO:954), TAR2h-171 (SEQ ID NO:955), TAR2h-172 (SEQ ID NO:956), TAR2h-173 (SEQ ID NO:957), TAR2h-174 (SEQ ID NO:958), TAR2h-176 (SEQ ID NO:959), TAR2h-178 (SEQ ID NO:960), TAR2h-201 (SEQ ID NO:961), TAR2h-202 (SEQ ID NO:962), TAR2h-203 (SEQ ID NO:963), TAR2h-204 (SEQ ID NO:964), TAR2h-185-25 (SEQ ID NO:965), TAR2h-154-10 SEQ ID NO:966), TAR2h-205 (SEQ ID NO:967), TAR2h-10 (SEQ ID NO:968), TAR2h-5 (SEQ ID NO:969), TAR2h-5d1 (SEQ ID NO:970), TAR2h-5d2 (SEQ ID NO:971), TAR2h-5d3 (SEQ ID NO:972), TAR2h-5d4 (SEQ ID NO:973), TAR2h-5d5 (SEQ ID NO:974), TAR2h-5d6 (SEQ ID NO:975), TAR2h-5d7 (SEQ ID NO:976), TAR2h-5d8 (SEQ ID NO:977), TAR2h-5d9 (SEQ ID NO:978), TAR2h-5d10 (SEQ ID NO:979), TAR2h-5d 11 (SEQ ID NO:980), TAR2h-5d12 (SEQ ID NO:981), and TAR2h-5d 13 (SEQ ID NO:982).

34. The ligand of claim 29 further comprising a half-life extending moiety.

35. The ligand of claim 34, wherein said half-life extending moiety is a polyalkylene glycol moiety, serum albumin or a fragment thereof, transferrin receptor or a transferrin-binding portion thereof, or an antibody or antibody fragment comprising a binding site for a polypeptide that enhances half-life in vivo.

36. The ligand of claim 35, wherein said half-life extending moiety is a polyethylene glycol moiety.

37. The ligand of claim 35, wherein said half-life extending moiety is an antibody or antibody fragment comprising a binding site for serum albumin or neonatal Fc receptor.

38. The ligand of claim 37, wherein said antibody or antibody fragment is an antibody fragment, and said antibody fragment is an immunoglobulin single variable domain.

39. The ligand of claim 38, wherein said immunoglobulin single variable domain competes for binding to human serum albumin with a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

40. The ligand of claim 39, wherein said immunoglobulin single variable domain binds human serum albumin comprises an amino acid sequence that has at least 90% amino acid sequence identity with the amino acid sequence of a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), DOM7r-14 (SEQ ID NO:748), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

41. An isolated nucleic acid encoding a dAb monomer of claim 1.

42. A recombinant nucleic acid encoding a dAb monomer of claim 1.

43. A vector comprising a nucleic acid encoding a dAb monomer claim 1.

44. The vector of claim 43, wherein said vector is an expression vector.

45. A host cell comprising a recombinant nucleic acid of claim 42.

46. A host cell comprising a vector of claim 43.

47. A method of producing a dAb monomer that has binding specificity for IL-1R1 and inhibits binding of IL-1 to the receptor but does not inhibit binding of IL-1 ra to IL-1R1, comprising maintaining a host cell of claim 45 under conditions suitable for expression of said recombinant nucleic acid.

48. A method of producing a dAb monomer that has binding specificity for IL-1R1 and inhibits binding of IL-1 to the receptor but does not inhibit binding of IL-1ra to IL-1R1, comprising maintaining a host cell of claim 46 under conditions suitable for expression of said vector.

49. A pharmaceutical composition comprising a dAb monomer of claim 1 and a physiologically acceptable carrier.

50. The pharmaceutical composition of claim 49 wherein said composition is for intravenous, intramuscular, intraperitoneal, intraarterial, intrathecal delivery device, intraarticular, or subcutaneous administration.

51. The pharmaceutical composition of claim 49 wherein said composition is for pulmonary, intranasal delivery device, vaginal, or rectal administration.

52. A drug delivery device comprising the pharmaceutical composition of claim 49.

53. The drug delivery device of claim 52 wherein said drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, and rectal delivery device.

54. The drug delivery device of claim 52 wherein said device is selected from the group consisting of a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, a catheter.

55. A method for treating an inflammatory disease comprising administering to a subject in need thereof a therapeutically effective amount of a dAb monomer of claim 1.

56. The method of claim 55 wherein said inflammatory disease is arthritis.

57-59. (canceled)

60. A method for treating an inflammatory disease, arthritis or respiratory disease comprising administering to a subject in need thereof a therapeutically effective amount of a domain antibody (dAb) monomer that is resistant to protease degradation.

61. The method of claim 60 wherein the dAb is administered via pulmonary administration.

62. The method of claim 60, wherein the dAb monomer is resistant to elastase.

63. The method of claim 60, wherein the dAb monomer is an immunoglobulin light chain variable domain.

64. The method of claim 63, wherein the dAb monomer is Vκ.

65. The method of claim 60, wherein the dAb has binding specificity for Interleukin-1 Receptor Type 1 (IL-1R1).

66. The method of claim 65, wherein the dAb monomer competes for binding to IL-1R1 with an anti-IL-1R1 dAb, wherein said anti-IL-1R1 dAb consists of an amino acid sequence selected from the group consisting of SEQ ID NO:1 through SEQ ID NO:349.

67. The method of claim 66, wherein the dAb monomer competes for binding to IL-1R1 with an anti-IL-1R1 dAb, wherein said anti-IL-1R1 dAb consists of an amino acid sequence selected from the group consisting consisting of SEQ ID NO:1 or SEQ ID NO:2.

68. An isolated nucleic acid encoding a ligand of claim 29.

69. A recombinant nucleic acid encoding ligand of claim 29.

70. A vector comprising a nucleic acid encoding a ligand of claim 29.

71. The vector of claim 70, wherein said vector is an expression vector.

72. A host cell comprising a recombinant nucleic acid of claim 69.

73. A host cell comprising a vector of claim 70.

74. A method of producing a ligand that has binding specificity for IL-1R1 and inhibits binding of IL-1 to the receptor but does not inhibit binding of IL-1ra to IL-1R1, comprising maintaining a host cell of claim 72 under conditions suitable for expression of said recombinant nucleic acid.

75. A method of producing a ligand that has binding specificity for IL-1R1 and inhibits binding of IL-1 to the receptor but does not inhibit binding of IL-1ra to IL-1R1, comprising maintaining a host cell of claim 73 under conditions suitable for expression of said vector.

76. A pharmaceutical composition comprising a ligand of claim 29 and a physiologically acceptable carrier.

77. The pharmaceutical composition of claim 76 wherein said composition is for intravenous, intramuscular, intraperitoneal, intraarterial, intrathecal delivery device, intraarticular, or subcutaneous administration.

78. The pharmaceutical composition of claim 76 wherein said composition is for pulmonary, intranasal delivery device, vaginal, or rectal administration.

79. A drug delivery device comprising the pharmaceutical composition of claim 76.

80. The drug delivery device of claim 79 wherein said drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, and rectal delivery device.

81. The drug delivery device of claim 80 wherein said device is selected from the group consisting of a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, a catheter.

82. A method for treating an inflammatory disease comprising administering to a subject in need thereof a therapeutically effective amount of a ligand of claim 29.

83. The method of claim 82 wherein said inflammatory disease is arthritis.
